(19) United States
(12) Patent Application Publication
(10) Pub. No.: US 2022/0089541 A1
(43) Pub. Date: Mar. 24, 2022

(54) Compounds for the Treatment of Kinase-Dependent Disorders

(71) Applicant: Exelixis, Inc., Alameda, CA (US)

(72) Inventors: Lynne Canne Bannen, Novato, CA (US); Faming Jiang, Castro Valley, CA (US); Kin Tso, San Francisco, CA (US); Xu Wei, Danville, CA (US)

(21) Appl. No.: 17/425,661

(22) PCT Filed: Jan. 24, 2020
(86) PCT No.: PCT/US2020/014979

§ 371 Date: Jul. 23, 2021

Related U.S. Application Data
(60) Provisional application No. 62/878,173, filed on Jul. 24, 2019.
(60) Provisional application No. 62/797,130, filed on Jan. 25, 2019.

(51) Int. Cl. C07D 215/22; C07D 215/48; C07D 401/12; C07D 471/04; A61P 35/00

(52) U.S. Cl. CPC C07D 215/22; C07D 215/48; A61P 35/00; C07D 471/04; C07D 401/12

(57) ABSTRACT

The present invention relates to compounds that modulate cellular activities such as proliferation, differentiation, programmed cell death, migration, and chemoinvasion, by modulating protein kinase enzymatic activity, and compositions thereof, and methods of using such compounds.

### CROSS REFERENCE TO RELATED APPLICATIONS

This application claims priority to U.S. Provisional Application Ser. No. 62/797,130, filed Jan. 25, 2019 and U.S. Provisional Application Ser. No. 62/878,173, filed Jul. 24, 2019, the entire contents of both of which are incorporated by reference herein.

### FIELD OF THE INVENTION

The present invention relates to compounds that modulate cellular activities such as proliferation, differentiation, programmed cell death, migration, and chemoinvasion, by modulating protein kinase enzymatic activity, and compositions thereof, and methods of using such compounds.

### BACKGROUND OF THE INVENTION

Human Axl belongs to the TAM subfamily of receptor tyrosine kinases that includes Mer. TAM kinases are characterized by an extracellular ligand binding domain consisting of two immunoglobulin-like domains and two fibronectin type III domains. Axl is overexpressed in a number of tumor cell types and was initially cloned from patients with chronic myelogenous leukemia. When overexpressed, Axl exhibits transforming potential. Axl signaling is believed to cause tumor growth through activation of proliferative and anti-apoptotic signaling pathways. Axl has been associated with cancers such as lung cancer, myeloid leukemia, uterine cancer, ovarian cancer, gliomas, melanoma, thyroid cancer, renal cell carcinoma, osteosarcoma, gastric cancer, prostate cancer, and breast cancer. The over-expression of Axl results in a poor prognosis for patients with the indicated cancers.

Activation of Mer, like Axl, conveys downstream signaling pathways that cause tumor growth and activation. Mer binds ligands such as the soluble protein Gas-6. Gas-6 binding to Mer induces autophosphorylation of Mer on its intracellular domain, resulting in downstream signal activation. Over-expression of Mer in cancer cells leads to increased metastasis, most likely by generation of soluble Mer extracellular domain protein as a decoy receptor. Tumor cells secrete a soluble form of the extracellular Mer receptor which reduces the ability of soluble Gas-6 ligand to activate Mer on endothelial cells, leading to cancer progression.

Therefore a need exists for compounds that inhibit TAM receptor tyrosine kinases such as Axl and Mer for the treatment of selected cancers.

### SUMMARY OF THE INVENTION

In one aspect, the present invention provides a compound of formula I′:

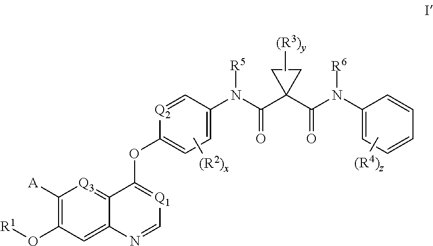

or a pharmaceutically acceptable salt thereof, wherein

A is a C₁₋₆ alkoxy, or C(O)NR⁷R⁸;

R¹ is C₁₋₆ alkyl or heterocycloalkyl-C₁₋₆ alkylene-;

R² is halo;

R³ is halo, OH, C₁₋₄ alkoxy, or CF₃;

R⁴ is halo;

one of R⁵ and R⁶ is —CHR′R″ and the other of R⁵ and R⁶ is H or —CHR′R″;

R⁷ and R⁸ are each independently H or a C₁₋₆ alkyl;

Each of R′ and R″ is independently selected from the group consisting of H, OH and C₁₋₆ alkoxy;

Q₁, Q₂, and Q₃ are each independently CH or N;

x is 0, 1, 2, 3, or 4;

y is 0, 1, 2, 3, or 4; and

z is 0, 1, 2, 3, 4, or 5.

In another aspect, the present invention provides a compound of formula I:

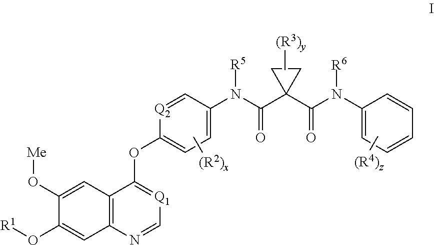

or a pharmaceutically acceptable salt thereof, wherein:

R¹ is C₁₋₆ alkyl or heterocycloalkyl-C₁₋₆ alkylene-;

R² is halo;

R³ is halo, OH, C₁₋₄ alkoxy, or CF₃;

R⁴ is halo;

one of R⁵ and R⁶ is —CHR′R″ and the other of R⁵ and R⁶ is H or —CHR′R″;

each of R′ and R″ is independently selected from the group consisting of H, OH and C₁₋₆ alkoxy;

Q₁ and Q₂ are each independently CH or N;

x is 0, 1, 2, 3, or 4;

y is 0, 1, 2, 3, or 4; and

z is 0, 1, 2, 3, 4, or 5.

Another aspect provides a compound of formula II:

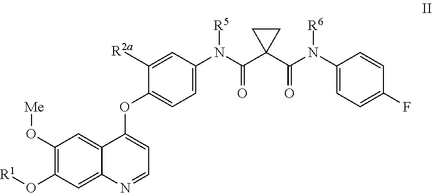

or a pharmaceutically acceptable salt thereof, wherein:

R¹ is C₁₋₆ alkyl or heterocycloalkyl-C₁₋₆ alkylene-;

R^{2a} is H or halo;

one of R⁵ and R⁶ is —CHR′R″ and the other of R⁵ and R⁶ is H or —CHR′R″; and

each of R′ and R″ is independently selected from the group consisting of H, OH and C₁₋₆ alkoxy.

Another aspect provides methods of using compounds of formula I′, formula I or formula II or pharmaceutically acceptable salts thereof for the treatment of a disease, disorder, or syndrome mediated at least in part by modulating in vivo activity of a protein kinase.

A further aspect provides processes for making compounds of formula I′, formula I and formula II.

These and other aspects and embodiments are described below.

### DETAILED DESCRIPTION OF THE INVENTION

### Abbreviations and Definitions

The following abbreviations and terms have the indicated meanings throughout:

| Abbreviation | Meaning |
|---|---|
| Ac | Acetyl |
| anhyd | Anhydrous |
| Aq | Aqueous |
| Boc | Tert-butoxycarbonyl |
| ° C. | Degrees Celsius |
| c- | Cyclo |
| calcd | Calculated |
| DCM | Dichloromethane |
| DIPEA | N,N-Diisopropylethylamine (Hünig's base) |
| DMF | N,N-Dimethylformamide |
| DMSO | Dimethyl sulfoxide |
| EI | Electron Impact ionization |
| eq or equiv | Equivalent |
| EtOAc | Ethyl acetate |
| Fmoc | Fluorenylmethyloxycarbonyl |
| g | Gram(s) |
| h or hr | Hour(s) |
| HATU | Hexafluorophosphate Azabenzotriazole |
| | Tetramethyl Uronium |
| HPLC | High pressure liquid chromatography |
| H₂ | Hydrogen |
| L | Liter(s) |
| M | Molar or molarity |
| MHz | Megahertz (frequency) |
| Min | Minute(s) |
| mL | Milliliter(s) |
| Mp | Melting point |
| μL | Microliter(s) |
| Mol | Mole(s) |
| MS | Mass spectral analysis |
| N₂ | Nitrogen |
| N | Normal or normality |
| nM | Nanomolar |
| NMR | Nuclear magnetic resonance spectroscopy |
| Pd/C | Palladium on carbon |
| RT | Room temperature |
| soln | Solution |
| THF | Tetrahydrofuran |

The symbol “—” means a single bond, and “═” means a double bond.

As used herein, the singular forms “a,” “an,” and “the” include plural reference unless the context clearly dictates otherwise.

When a variable is defined generically, with a number of possible substituents, each individual radical can be defined with our without the bond. For example, if R^{z} can be hydrogen, this can be indicated as “—H” or “H” in the definition of R^{z}.

When chemical structures are depicted or described, unless explicitly stated otherwise, all carbons are assumed to have hydrogen substitution to conform to a valence of four. For example, in the structure on the left-hand side of the schematic below, there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

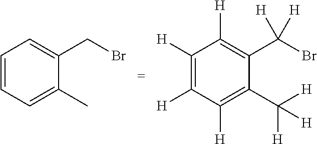

As used herein, a wavy line, [symbol] can indicate the attachment point of a chemical moiety. For example, in the structure,

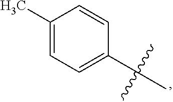 [SMILES: *c1ccc(C)cc1]

the phenyl group is attached to the rest of the molecule at the position para to the methyl group.

If a group “R” is depicted as “floating” on a ring system, as for example in the formula.

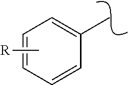

then, unless otherwise defined, a substituent “R” may reside on any atom of the ring system, assuming replacement of a depicted, implied, or expressly defined hydrogen from one of the ring atoms, so long as a stable structure is formed.

When a group “R” is depicted as existing on a ring system containing saturated carbons, for example in the formula:

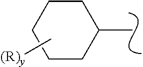

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group, there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

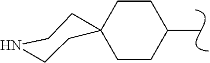

“Halogen” or “halo” refers to fluorine, chlorine, bromine, or iodine.

The term “Cₙ₋ₘ” or “Cₙ-Cₘ” indicates a range which includes the endpoints, wherein n and m are integers and indicate the number of carbons. Examples include C₁₋₄, C₁-C₄, C₁₋₆, C₁-C₆, and the like.

“Alkyl” refers to a branched or straight hydrocarbon chain of one to eight carbon atoms, such as methyl, ethyl, n-propyl, isopropyl, n-butyl, isobutyl, sec-butyl, t-butyl, pentyl, hexyl, and heptyl. The term “Cₙ₋ₘ alkyl” or (Cₙ-Cₘ) alkyl, refers to an alkyl group having n to m carbon atoms.

“Alkylene” refers to an optionally substituted bivalent saturated aliphatic radical having from 1 to 10 carbon atoms, 1 to 8 carbon atoms, 1 to 6 carbon atoms, 1 to 4 carbon atoms, or 1 to 2 carbon atoms. The term “Cn-m alkylene” refers to an alkylene group having n to m carbon atoms. Examples of alkylene groups include, but are not limited to, methylene, ethan-1,2-diyl, propan-1,3-diyl, propan-1,2-diyl, butan-1,4-diyl, butan-1,3-diyl, butan-1,2-diyl, 2-methyl-propan-1,3-diyl and the like.

As used herein, an “alkoxy” group refers to an alkyl-O— group where “alkyl” has been defined previously.

As used herein, “heterocycloalkyl” or “heterocyclo” refer to a non-aromatic ring or ring system, which may optionally contain one or more alkenylene groups as part of the ring structure, which has at least one heteroatom ring member independently selected from boron, nitrogen, sulfur, oxygen, and phosphorus, and which has 4-14 ring members, 4-10 ring members, 4-7 ring members, or 4-6 ring members. Included within the term “heterocycloalkyl” are monocyclic 4-, 5-, 6-, and 7-membered heterocycloalkyl groups.

Heterocycloalkyl groups can include mono- or bicyclic or polycyclic (for example, having two or three fused or bridged rings) ring systems or spirocycles. In some embodiments, the heterocycloalkyl group is a monocyclic group having 1, 2, or 3 heteroatoms independently selected from nitrogen, sulfur, and oxygen. Ring-forming carbon atoms and heteroatoms of a heterocycloalkyl group can be optionally oxidized to form an oxo or sulfido group or other oxidized linkage (for example, C(O), S(O), C(S), S(O)₂, N-oxide, etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, for example, benzo or thienyl derivatives of piperidine, morpholine, azepine, etc. A heterocycloalkyl group containing a fused aromatic ring can be attached through any ring-forming atom, including a ring-forming atom of the fused aromatic ring. Examples of heterocycloalkyl groups include azetidinyl, azepanyl, dihydrobenzofuranyl, dihydrofuranyl, dihydropyranyl, morpholino, 3-oxa-9-azaspiro[5.5]undecanyl, 1-oxa-8-azaspiro[4.5]decanyl, piperidinyl, piperazinyl, oxopiperazinyl, pyranyl, pyrrolidinyl, quinuclidinyl, tetrahydrofuranyl, tetrahydropyranyl, 1,2,3,4-tetrahydroquinolinyl, tropanyl, 4,5,6,7-tetrahydrothiazolo[5,4-c]pyridinyl, and thiomorpholino.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (for example, chloro, bromo, iodo), —OR^{LG} (when the O atom is attached to a carbonyl group), —O(C═O)R^{LG}, or —O(SO)₂R^{LG} (for example, tosyl, mesyl, besyl), wherein R^{LG} is optionally substituted alkyl, optionally substituted aryl, or optionally substituted heteroaryl. In certain embodiments, the leaving group is a halogen.

“Yield” for each of the reactions described herein is expressed as a percentage of the theoretical yield.

“Patient” for the purposes of the present invention includes humans and any other animals, particularly mammals, and other organisms. Thus the methods are applicable to both human therapy and veterinary applications. In a preferred embodiment the patient is a mammal, and in a most preferred embodiment the patient is human. Examples of the preferred mammals include mice, rats, other rodents, rabbits, dogs, cats, swine, cattle, sheep, horses, and primates.

“Kinase-dependent diseases or conditions” refer to pathologic conditions that depend on the activity of one or more kinases. Kinases either directly or indirectly participate in the signal transduction pathways of a variety of cellular activities including proliferation, adhesion, migration, differentiation, and invasion. Diseases associated with kinase activities include tumor growth, the pathologic neovascularization that supports solid tumor growth, and associated with other diseases where excessive local vascularization is involved such as ocular diseases (diabetic retinopathy, age-related macular degeneration, and the like) and inflammation (psoriasis, rheumatoid arthritis, and the like).

“Therapeutically effective amount” is an amount of a compound of the invention that, when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

“Cancer” refers to cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Head and neck: squamous cell carcinomas of the head and neck, laryngeal and hypopharyngeal cancer, nasal cavity and paranasal sinus cancer, nasopharyngeal cancer, salivary gland cancer, oral and or pharyngeal cancer; Lung: bronchogenic carcinoma (squamous cell, undifferentiated small cell, undifferentiated large cell, adenocarcinoma, non-small cell lung cancer), alveolar (bronchiolar) carcinoma, bronchial adenoma, sarcoma, lymphoma, chondromatous hamartoma, mesothelioma; Colon: colorectal cancer, adenocarcinoma, gastrointestinal stromal tumors, lymphoma, carcinoids, Turcot Syndrome; Gastrointestinal: gastric cancer, gastroesophageal junction adenocarcinoma, esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Breast: metastatic breast cancer, ductal carcinoma in situ, invasive ductal carcinoma, tubular carcinoma, medullary carcinoma, mucinous carcinoma, lobular carcinoma in situ, triple negative breast cancer; Genitourinary tract: kidney (adenocarcinoma, Wilm's tumor [nephroblastoma], lymphoma, leukemia, renal cell carcinoma), bladder and urethra (squamous cell carcinoma, transitional cell carcinoma, adenocarcinoma, urothelial carcinoma), prostate (adenocarcinoma, sarcoma, castrate resistant prostate cancer), testis (seminoma, teratoma, embryonal carcinoma, teratocarcinoma, choriocarcinoma, sarcoma, interstitial cell carcinoma, fibroma, fibroadenoma, adenomatoid tumors, lipoma), clear cell carcinoma, papillary carcinoma; Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma, and giant cell tumors; Thyroid: medullary thyroid cancer, differentiated thyroid cancer, papillary thyroid cancer, follicular thyroid cancer, hurthle cell cancer, and anaplastic thyroid cancer; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial cancer), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

The term, “compound,” as used herein is meant to include all stereoisomers, geometric isomers, tautomers, and isotopes of the structures depicted. The term is also meant to refer to compounds of the inventions, regardless of how they are prepared, for example, synthetically, through biological process (for example, metabolism or enzyme conversion), or a combination thereof.

Compounds of the invention can also include all isotopes of atoms occurring in the intermediates or final compounds. Isotopes include those atoms having the same atomic number but different mass numbers. For example, isotopes of hydrogen include tritium and deuterium.

Any one of the process steps or sequences disclosed and/or claimed herein can be performed under an inert gas atmosphere, more particularly under argon or nitrogen. In addition, the methods of the present invention may be carried out as semi-continuous or continuous processes, more preferably as continuous processes.

Moreover, many of the process steps and sequences that are described herein can be telescoped.

In general, the nomenclature used in this Application is based on naming conventions adopted by the International Union of Pure and Applied Chemistry (IUPAC).

Chemical structures shown herein were prepared using CHEMDRAW®. Any open valency appearing on a carbon, oxygen, or nitrogen atom in the structures herein indicates the presence of a hydrogen atom.

### Embodiments of the Invention

One aspect provides a compound of formula T:

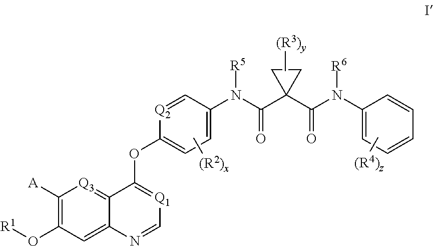

or a pharmaceutically acceptable salt thereof, wherein

A is a C₁₋₆ alkoxy, or C(O)NR⁷R⁸;

R¹ is C₁₋₆ alkyl or heterocycloalkyl-C₁₋₆ alkylene-;

R² is halo;

R³ is halo, OH, C₁₋₄ alkoxy, or CF₃;

R⁴ is halo;

one of R⁵ and R⁶ is —CHR′R″ and the other of R⁵ and R⁶ is H or —CHR′R″;

R⁷ and R⁸ are each independently H or a C₁₋₆ alkyl;

each of R′ and R″ is independently selected from the group consisting of H, OH and C₁₋₆ alkoxy;

Q₁, Q₂, and Q₃ are each independently CH or N;

x is 0, 1, 2, 3, or 4;

y is 0, 1, 2, 3, or 4; and

z is 0, 1, 2, 3, 4, or 5.

In some embodiments of this aspect, R¹ is C₁₋₆ alkyl or

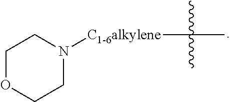

In some embodiments, R¹ is C₁₋₆ alkyl. In some embodiments, R¹ is methyl. In other embodiments, R¹ is

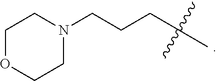 [SMILES: *CCCN1CCOCC1]

In other embodiments, R¹ is

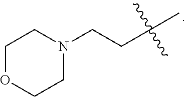 [SMILES: *CCN1CCOCC1]

In some embodiments of this aspect, R² is F. In some embodiments, R³ is halo. In some embodiments, R³ is F. In some embodiments, R⁴ is F. In some embodiments, R², R³, and R⁴, are each independently F.

In some embodiments of this aspect, x is 0 or 1. In some embodiments, y is 0 or 1. And in some embodiments, z is 0 or 1. In some embodiments, x, y, and z are each independently 0 or 1. In some embodiments, y is 0. In some embodiments, z is 1. In some embodiments, y is 0 and z is 1.

In some embodiments of this aspect, one of R⁵ and R⁶ is —CHR′R″ and the other is H. In some embodiments of this aspect, R⁵ is —CHR′R″ and R⁶ is H. In other embodiments of this aspect, R⁶ is —CHR′R″ and R⁵ is H. In other embodiments of this aspect, R⁵ and R⁶ are each independently —CHR′R″. In some embodiments, R⁵ is methyl. In some embodiments, R⁵ is methyl and R⁶ is H. In some embodiments, R⁶ is methyl. In some embodiments, R⁶ is methyl and R⁵ is H. In some embodiments, R⁵ and R⁶ are each methyl. In some embodiments, R⁵ is —CH₂OH. In some embodiments, R⁶ is —CH₂OH. In some embodiments, R⁵ is —CH₂O—(C₁-C₆ alkyl). In some embodiments, R⁶ is s —CH₂O—(C₁-C₆ alkyl). In some embodiments, R⁵ is —CH₂OCH₃. In some embodiments, R⁶ is —CH₂OCH₃.

In some embodiments of this aspect, Q₁ is CH. In some embodiments of this aspect Q₂ is CH. In some embodiments, Q₁ and Q₂ are each CH. In some embodiments of this aspect, Q₁ is CH and Q₂ is N. In other embodiments, Q₁ is N and Q₂ is CH. In yet other embodiments, Q₁ and Q₂ are each N.

In some embodiments of this aspect, A is a C₁₋₆ alkoxy. In another embodiment, A is methoxy, ethoxy, n-propoxy, isopropoxy, butoxy, or t-butoxy. In a further embodiment, A is methoxy.

In some embodiments of this aspect, A is C(O)NR⁷R⁸, wherein R⁷ and R⁸ are each independently H or a C₁₋₆ alkyl.

In one embodiment, one of R⁷ and R⁸ is H, and the other is a C₁₋₆ alkyl. In another embodiment, both of R⁷ and R⁸ are H. In another embodiment, both of R⁷ and R⁸ are a C₁₋₆ alkyl.

In some embodiments, each C₁₋₄ alkyl is independently methyl, ethyl, propyl, isopropyl, butyl, or t-butyl. In a further embodiment, each C₁₋₄ alkyl is methyl.

In some embodiments, Q₃ is CH. In some embodiments, Q₃ is N.

One aspect provides a compound of formula I:

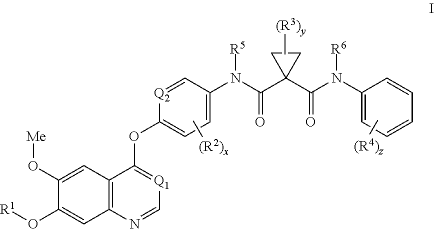

or a pharmaceutically acceptable salt thereof, wherein

R¹ is C₁₋₆ alkyl or heterocycloalkyl-C₁₋₆ alkylene-;

R² is halo;

R³ is halo, OH, C₁₋₄ alkoxy, or CF₃;

R⁴ is halo;

one of R⁵ and R⁶ is —CHR′R″ and the other of R⁵ and R⁶ is H or —CHR′R″;

each of R′ and R″ is independently selected from the group consisting of H, OH and C₁₋₆ alkoxy;

Q₁ and Q₂ are each independently CH or N;

x is 0, 1, 2, 3, or 4;

y is 0, 1, 2, 3, or 4; and

z is 0, 1, 2, 3, 4, or 5.

In some embodiments of this aspect, R¹ is C₁₋₆ alkyl or

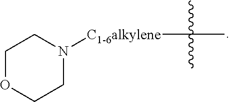

In some embodiments, R¹ is C₁₋₆ alkyl. In some embodiments, R¹ is methyl. In other embodiments, R¹ is

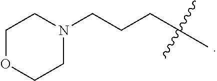 [SMILES: *CCCN1CCOCC1]

In other embodiments, R¹ is

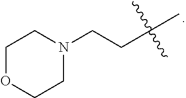 [SMILES: *CCN1CCOCC1]

In some embodiments of this aspect, R² is F. In some embodiments, R³ is halo. In some embodiments, R³ is F. In some embodiments, R⁴ is F. In some embodiments, R², R³, and R⁴, are each independently F.

In some embodiments of this aspect, x is 0 or 1. In some embodiments, y is 0 or 1.

And in some embodiments, z is 0 or 1. In some embodiments, x, y, and z are each independently 0 or 1. In some embodiments, y is 0. In some embodiments, z is 1. In some embodiments, y is 0 and z is 1.

In some embodiments of this aspect, one of R⁵ and R⁶ is —CHR′R″ and the other is H. In some embodiments of this aspect, R⁵ is —CHR′R″ and R⁶ is H. In other embodiments of this aspect, R⁶ is —CHR′R″ and R⁵ is H. In other embodiments of this aspect, R⁵ and R⁶ are each independently —CHR′R″. In some embodiments, R⁵ is methyl. In some embodiments, R⁵ is methyl and R⁶ is H. In some embodiments, R⁶ is methyl. In some embodiments, R⁶ is methyl and R⁵ is H. In some embodiments, R⁵ and R⁶ are each methyl. In some embodiments, R⁵ is —CH₂OH. In some embodiments, R⁶ is —CH₂OH. In some embodiments, R⁵ is —CH₂O—(C₁-C₆ alkyl). In some embodiments, R⁶ is s —CH₂O—(C₁-C₆ alkyl). In some embodiments, R⁵ is —CH₂OCH₃. In some embodiments, R⁶ is —CH₂OCH₃.

In some embodiments of this aspect, Q₁ is CH. In some embodiments of this aspect Q₂ is CH. In some embodiments, Q₁ and Q₂ are each CH. In some embodiments of this aspect, Q₁ is CH and Q₂ is N. In other embodiments, Q₁ is N and Q₂ is CH. In yet other embodiments, Q₁ and Q₂ are each N.

In some embodiments of this aspect, the compound of formula I is a compound of formula IA:

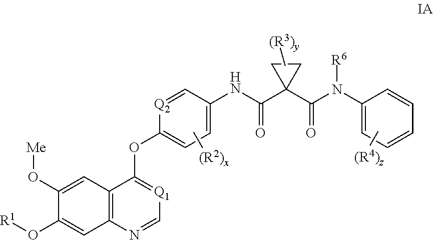

or a pharmaceutically acceptable salt thereof, wherein R¹, R², R³, R⁴, Q₁, Q₂, x, y and z are as defined in any of the embodiments of formula I; and R⁶ is —CHR′R″ (wherein R′ and R″ are as defined in any of the embodiments of formula I). In some embodiments of this embodiment, R⁶ is methyl. In other embodiments of this embodiment, R⁶ is —CH₂OH. In other embodiments of this embodiment, R⁶ is —CH₂OCH₃.

In some embodiments of this aspect, the compound of formula I is a compound of formula IA-1:

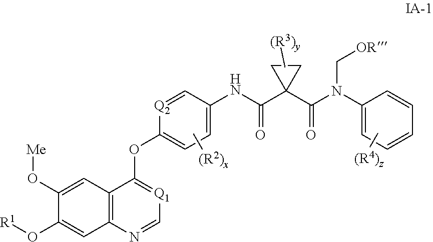

or a pharmaceutically acceptable salt thereof, wherein R¹, R², R³, R⁴, Q₁, Q₂, x, y and z are as defined in any of the embodiments of formula I and R′″ is H or methyl. In some embodiments of this embodiment, R′″ is H.

In some embodiments of this aspect, the compound of formula I is a compound of formula IB:

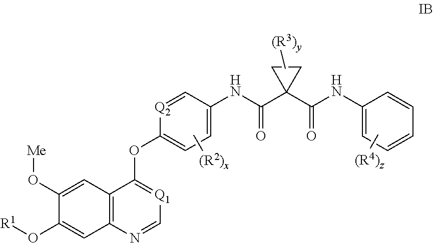

or a pharmaceutically acceptable salt thereof, wherein R¹, R², R³, R⁴, Q₁, Q₂, x, y and z are as defined in any of the embodiments of formula I; and R⁵ is —CHR′R″ (wherein R′ and R″ are as defined in any of the embodiments of formula I). In some embodiments of this embodiment, R⁵ is methyl. In other embodiments of this embodiment, R⁵ is —CH₂OH. In other embodiments of this embodiment, R⁵ is —CH₂OCH₃.

In some embodiments of this aspect, the compound of formula I is a compound of formula IB-1:

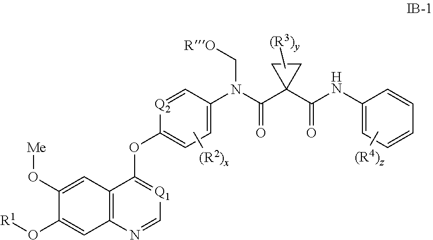

or a pharmaceutically acceptable salt thereof, wherein R¹, R², R³, R⁴, Q₁, Q₂, x, y and z are as defined in any of the embodiments of formula I and R″″ is H or methyl. In some embodiments of this embodiment, R″″ is H.

Another aspect provides a compound of formula II:

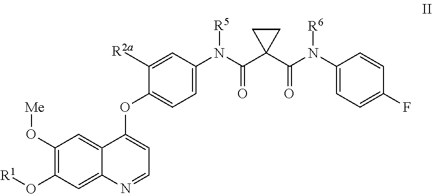

or a pharmaceutically acceptable salt thereof, wherein

R¹ is C₁₋₆ alkyl or heterocycloalkyl-C₁₋₆ alkylene-;

R^{2a} is H or halo;

one of R⁵ and R⁶ is —CHR′R″ and the other of R⁵ and R⁶ is H or —CHR′R″; and

each of R′ and R″ is independently selected from the group consisting of H, OH and C₁₋₆ alkoxy.

In some embodiments of this aspect, R¹ is C₁₋₆ alkyl or

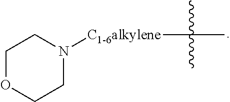

In some embodiments, R¹ is C₁₋₆ alkyl. In some embodiments, R¹ is methyl. In other embodiments, R¹ is

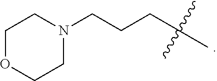 [SMILES: *CCCN1CCOCC1]

In other embodiments, R¹ is

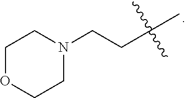 [SMILES: *CCN1CCOCC1]

In some embodiments of this aspect, R^{2a} is H or F. In some embodiments, R^{2a} is H. In other embodiments, R^{2a} is F.

In some embodiments of this aspect, one of R⁵ and R⁶ is —CHR′R″ and the other is H. In some embodiments of this aspect, R⁵ is —CHR′R″ and R⁶ is H. In other embodiments of this aspect, R⁶ is —CHR′R″ and R⁵ is H. In other embodiments of this aspect, R⁵ and R⁶ are each independently —CHR′R″. In some embodiments, R⁵ is methyl. In some embodiments, R⁵ is methyl and R⁶ is H. In some embodiments, R⁶ is methyl. In some embodiments, R⁶ is methyl and R⁵ is H. In some embodiments, R⁵ and R⁶ are each methyl. In some embodiments, R⁵ is —CH₂OH. In some embodiments, R⁶ is —CH₂OH. In some embodiments, R⁵ is —CH₂OCH₃. In some embodiments, R⁶ is —CH₂OCH₃.

In some embodiments of this aspect, the compound of formula II is a compound of formula II A:

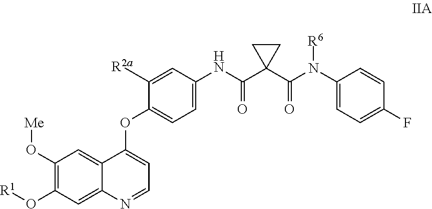

or a pharmaceutically acceptable salt thereof, wherein R¹ and R^{2a} are as defined in any of the embodiments of formula II, and R⁶ is —CHR′R″ (wherein R″ and R″ are as defined in any of the embodiments of formula II). In some embodiments of this embodiment, R⁶ is methyl. In other embodiments of this embodiment, R⁶ is —CH₂OH. In other embodiments of this embodiment, R⁶ is —CH₂OCH₃. In some embodiments of this aspect, the compound of formula II is a compound of formula IIA-1:

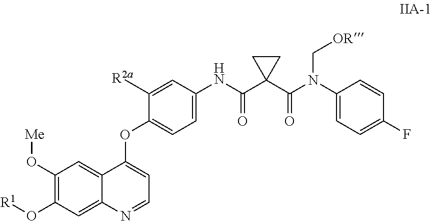

or a pharmaceutically acceptable salt thereof, wherein R¹ and R^{2a} are as defined in any of the embodiments of formula I or II and R′″ is H or methyl. In some embodiments of this embodiment, R′″ is H. In some embodiments of this embodiment, R′″ is methyl.

In some embodiments of this aspect, the compound of formula II is a compound of formula IIB:

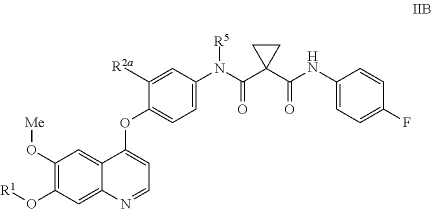

or a pharmaceutically acceptable salt thereof, wherein R¹ and R^{2a} are as defined in any of the embodiments of formula II, and R⁵ is —CHR′R″ (wherein R″ and R″ are as defined in any of the embodiments of formula II). In some embodiments of this embodiment, R⁵ is methyl. In other embodiments of this embodiment, R⁵ is —CH₂OH. In other embodiments of this embodiment, R⁵ is —CH₂OCH₃.

In some embodiments of this aspect, the compound of formula II is a compound of formula IIB-1:

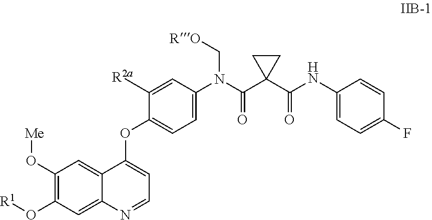

or a pharmaceutically acceptable salt thereof, wherein R¹ and R^{2a} are as defined in any of the embodiments of formula I or II and R″″ is H or methyl. In some embodiments of this embodiment, R″″ is H.

In some embodiments, the compound of formula I′ is a compound of formula Ilia:

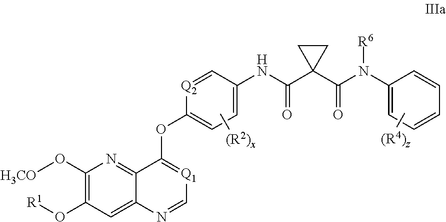

or a pharmaceutically acceptable salt thereof, wherein R¹, R², R⁴, R⁶, Q₁, Q₂, x and z are as defined in any of the embodiments of formula I′.

In some embodiments, the compound of formula I′ is a compound of formula IIIb:

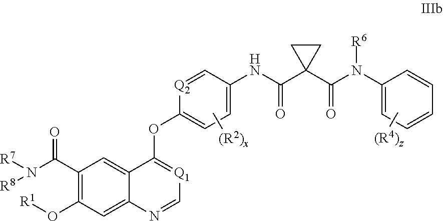

or a pharmaceutically acceptable salt thereof, wherein R¹, R², R⁴, R⁶, R⁷, R⁸, Q₁, Q₂, x and z are as defined in any of the embodiments of formula I′.

In some embodiments, A is a C₁₋₆ alkoxy. In a further embodiment, A is methoxy, ethoxy, n-propoxy, isopropoxy, butoxy, or t-butoxy. In still a further embodiment, A is methoxy.

In some other embodiments, A is C(O)NR⁷R⁸.

In another embodiment, one of R⁷ and R⁸ is H, and the other is a C₁₋₆ alkyl. In a further embodiment, one of R⁷ and R⁸ is H, and the other is methyl.

In some other embodiments, both R⁷ and R⁸ are H.

In some embodiments, R² is halo. In a further embodiment, R² is F.

In some embodiments, R⁴ is F.

In one embodiment, the moiety

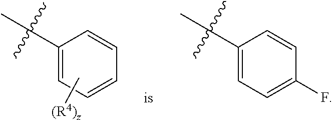

In some embodiments, Q₁, Q₂, and Q₃ are each CH.

In some other embodiments, Q₁, and Q₃ are each CH, and Q₂ is N.

In some other embodiments, Q₁, and Q₂ are each CH, and Q₃ is N.

In another aspect, the invention provides a compound of formula I′, I, or II or a pharmaceutically acceptable salt thereof, as provided in Table 1.

TABLE 1
Compounds of Formula I′, I, or II

| Cpd. | | |
|---|---|---|
| # | Structure | Name |
| 9 | 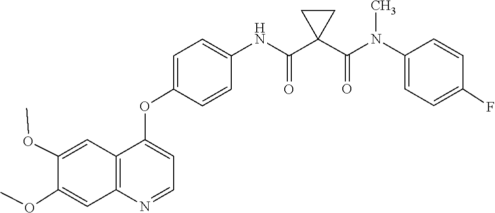 [SMILES: COc1cc2nccc(Oc3ccc(NC(=O)C4(C(=O)N(C)c5ccc(F)cc5)CC4)cc3)c2cc1OC] | N-(4-((6,7-dimethoxyquinolin- 4-yl)oxy)phenyl)-N-(4- fluorophenyl)-N- methylcyclopropane-1,1- dicarboxamide |
| 10 | 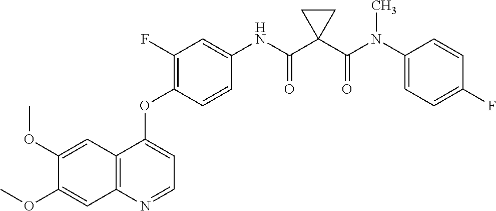 [SMILES: COc1cc2nccc(Oc3ccc(NC(=O)C4(C(=O)N(C)c5ccc(F)cc5)CC4)cc3F)c2cc1OC] | N-(4-((6,7-dimethoxyquinolin- 4-yl)oxy)-3-fluorophenyl)-N- (4-fluorophenyl)-N- methylcyclopropane-1,1- dicarboxamide |
| 11 | 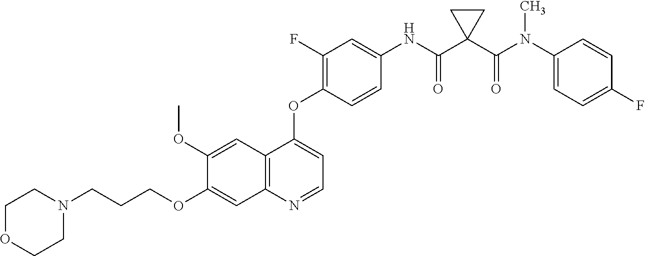 [SMILES: COc1cc2c(Oc3ccc(NC(=O)C4(C(=O)N(C)c5ccc(F)cc5)CC4)cc3F)ccnc2cc1OCCCN1CCOCC1] | N-(3-fluoro-4-((6-methoxy-7- (3- morpholinopropoxy)quinolin- 4-yl)oxy)phenyl)-N-(4- fluorophenyl)-N- methylcyclopropane-1,1- dicarboxamide |
| 12 | 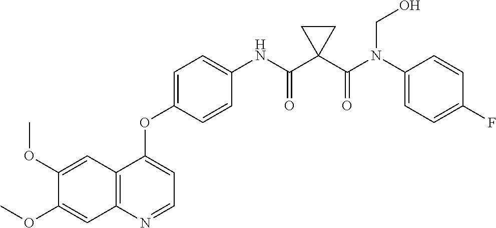 [SMILES: COc1cc2nccc(Oc3ccc(NC(=O)C4(C(=O)N(CO)c5ccc(F)cc5)CC4)cc3)c2cc1OC] | N-(4-((6,7-dimethoxyquinolin- 4-yl)oxy)phenyl)-N-(4- fluorophenyl)-N- (hydroxymethyl)cyclopropane- 1,1-dicarboxamide |
| 13 | 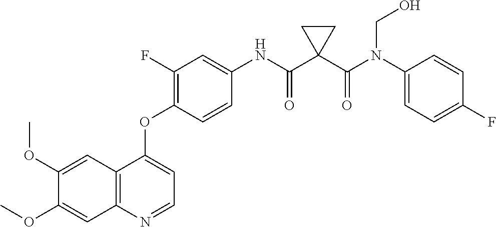 [SMILES: COc1cc2nccc(Oc3ccc(NC(=O)C4(C(=O)N(CO)c5ccc(F)cc5)CC4)cc3F)c2cc1OC] | N-(4-((6,7-dimethoxyquinolin- 4-yl)oxy)-3-fluorophenyl)-N- (4-fluorophenyl)-N- (hydroxymethyl)cyclopropane- 1,1-dicarboxamide |
| 14 | 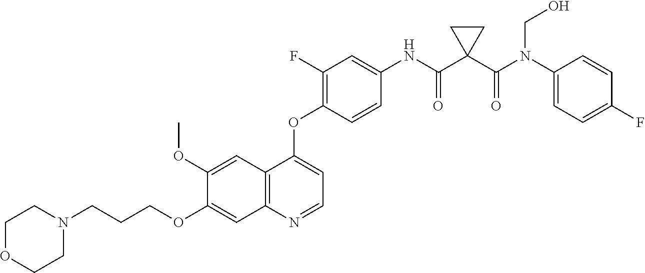 [SMILES: COc1cc2c(Oc3ccc(NC(=O)C4(C(=O)N(CO)c5ccc(F)cc5)CC4)cc3F)ccnc2cc1OCCCN1CCOCC1] | N-(3-fluoro-4-((6-methoxy-7- (3- morpholinopropoxy)quinolin- 4-yl)oxy)phenyl)-N-(4- fluorophenyl)-N- (hydroxymethyl)cyclopropane- 1,1-dicarboxamide |
| 34 | 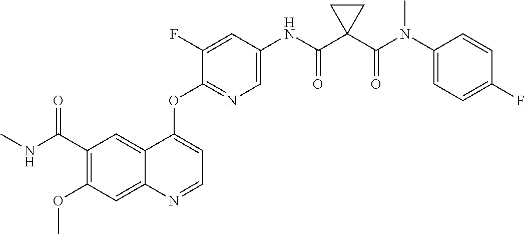 [SMILES: CNC(=O)c1cc2c(Oc3ncc(NC(=O)C4(C(=O)N(C)c5ccc(F)cc5)CC4)cc3F)ccnc2cc1OC] | 1-N-[5-fluoro-6-[7-methoxy-6- (methylcarbamoyl)quinolin-4- yl]oxypyridin-3-yl]-1-N′-(4- fluorophenyl)-1-N′- methylcyclopropane-1,1- dicarboxamide |
| 34b | 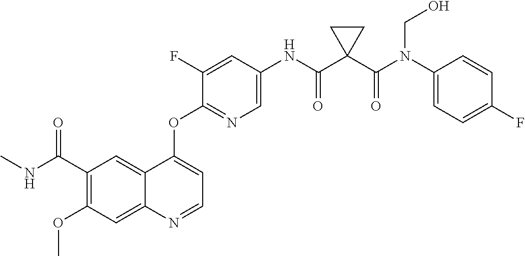 [SMILES: CNC(=O)c1cc2c(Oc3ncc(NC(=O)C4(C(=O)N(CO)c5ccc(F)cc5)CC4)cc3F)ccnc2cc1OC] | N-(5-fluoro-6-((7-methoxy-6- (methylcarbamoyl)quinolin-4- yl)oxy)pyridin-3-yl)-N-(4- fluorophenyl)-N- (hydroxymethyl)cyclopropane- 1,1-dicarboxamide |

| Cpd. | | |
|---|---|---|
| # | Structure | Name |
| 35 | 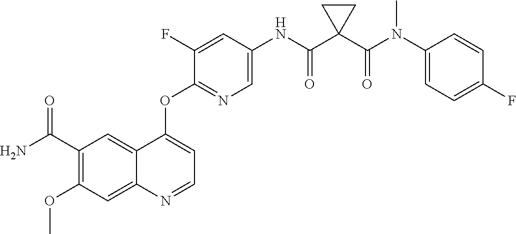 [SMILES: COc1cc2nccc(Oc3ncc(NC(=O)C4(C(=O)N(C)c5ccc(F)cc5)CC4)cc3F)c2cc1C(N)=O] | 1-N-[6-(6-carbamoyl-7- methoxyquinolin-4-yl)oxy-5- fluoropyridin-3-yl]-1-N′-(4- fluorophenyl)-1-N′- methylcyclopropane-1,1- dicarboxamide |
| 35b | 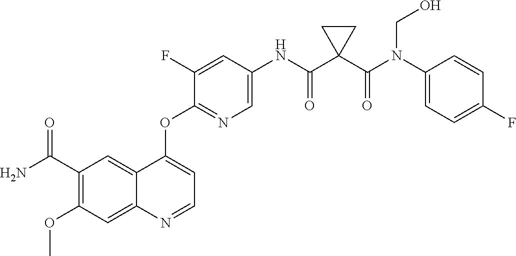 [SMILES: COc1cc2nccc(Oc3ncc(NC(=O)C4(C(=O)N(CO)c5ccc(F)cc5)CC4)cc3F)c2cc1C(N)=O] | N-(6-((6-carbamoyl-7- methoxyquinolin-4-yl)oxy)-5- fluoropyridin-3-yl)-N-(4- fluorophenyl)-N- (hydroxymethyl)cyclopropane- 1,1-dicarboxamide |
| 44 | 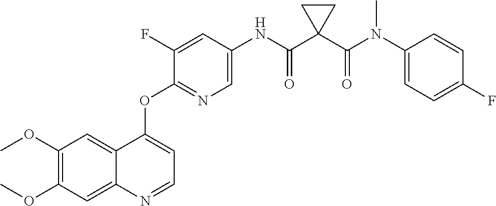 [SMILES: COc1cc2nccc(Oc3ncc(NC(=O)C4(C(=O)N(C)c5ccc(F)cc5)CC4)cc3F)c2cc1OC] | 1-N-[4-[(6,7-dimethoxy-1,5- naphthyridin-4-yl)oxy]-3- fluorophenyl]-1-N′-(4- fluorophenyl)-1-N′- methylcyclopropane-1,1- dicarboxamide |
| 44b | 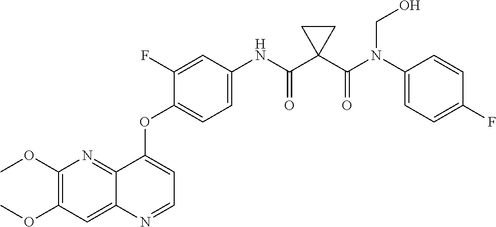 [SMILES: COc1cc2nccc(Oc3ccc(NC(=O)C4(C(=O)N(CO)c5ccc(F)cc5)CC4)cc3F)c2nc1OC] | N-(4-((6,7-dimethoxy-1,5- naphthyridin-4-yl)oxy)-3- fluorophenyl)-N-(4- fluorophenyl)-N- (hydroxymethyl)cyclopropane- 1,1-dicarboxamide |
| 49 | 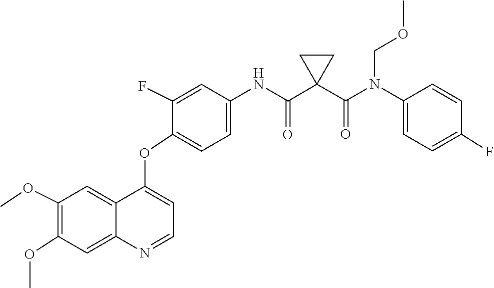 [SMILES: COCN(C(=O)C1(C(=O)Nc2ccc(Oc3ccnc4cc(OC)c(OC)cc34)c(F)c2)CC1)c1ccc(F)cc1] | 1-N-[4-(6,7- dimethoxyquinolin-4- yl)oxyphenyl]-1-N′-(4- fluorophenyl)-1-N′- (methoxymethyl)cyclopropane- 1,1-dicarboxamide |
| 51 | 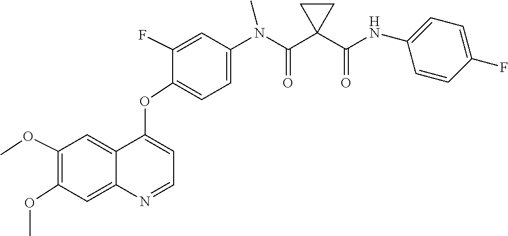 [SMILES: COc1cc2nccc(Oc3ccc(N(C)C(=O)C4(C(=O)Nc5ccc(F)cc5)CC4)cc3F)c2cc1OC] | 1-N′-[4-(6,7- dimethoxyquinolin-4-yl)oxy-3- fluorophenyl]-1-N-(4- fluorophenyl)-1-N′- methylcyclopropane-1,1- dicarboxamide |

General Administration

Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as, for example, tablets, suppositories, pills, soft elastic and hard gelatin capsules, powders, solutions, suspensions, aerosols, and the like, preferably in unit dosage forms suitable for simple administration of precise dosages.

The compositions will include a conventional pharmaceutical carrier or excipient and a compound of the invention as the/an active agent, and, in addition, may include other medicinal agents, pharmaceutical agents, carriers, adjuvants, and the like. Compositions of the invention may be used in combination with anticancer or other agents that are generally administered to a patient being treated for cancer. Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate, and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, and the like.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), suitable mixtures thereof, vegetable oils (such as olive oil), and injectable organic esters such as ethyl oleate. Proper fluidity can be maintained, for example, by the use of a coating such as lecithin, by the maintenance of the required particle size in the case of dispersions, and by the use of surfactants.

One preferable route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate, and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, and the like, a compound(s) of the invention, or a pharmaceutically acceptable salt thereof, and optional pharmaceutical adjuvants in a carrier, such as, for example, water, saline, aqueous dextrose, glycerol, ethanol, and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, and dimethylformamide; oils, in particular, cottonseed oil, groundnut oil, corn germ oil, olive oil, castor oil, and sesame oil, glycerol, tetrahydrofurfuryl alcohol, polyethyleneglycols, and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol, and sorbitan esters, microcrystalline cellulose, aluminum metahydroxide, bentonite, agar-agar, and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol, or a suppository wax, which are solid at ordinary temperatures but liquid at body temperature and therefore melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode, and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

Combination Therapy

A compound as disclosed herein can be administered as a single therapy or in combination (“co-administered”) with one or more additional therapies for the treatment of a disease or disorder, for instance a disease or disorder associated with hyper-proliferation such as cancer. Therapies that may be used in combination with a compound disclosed herein include: (i) surgery; (ii) radiotherapy (for example, gamma radiation, neutron beam radiotherapy, electron beam radiotherapy, proton therapy, brachytherapy, and systemic radioactive isotopes); (iii) endocrine therapy; (iv) adjuvant therapy, immunotherapy, CAR T-cell therapy; and (v) other chemotherapeutic agents.

The term “co-administered” (“co-administering”) refers to either simultaneous administration, or any manner of separate sequential administration, of a compound of the invention or a salt thereof, and a further active pharmaceutical ingredient or ingredients, including cytotoxic agents and radiation treatment. If the administration is not simultaneous, the compounds are administered in a close time proximity to each other. Furthermore, it does not matter if the compounds are administered in the same dosage form, e.g. one compound may be administered topically and another compound may be administered orally.

Typically, any agent that has activity against a disease or condition being treated may be co-administered. Examples of such agents for cancer treatment can be found, for instance, at https://www.cancer.gov/about-cancer/treatment/drugs (last visited Jan. 22, 2019) and in publically available sources such as Cancer Principles and Practice of Oncology by V. T. Devita and S. Heilman (editors), 11^{th} edition (2018), Lippincott Williams & Wilkins Publishers. A person of ordinary skill in the art would be able to discern which combinations of agents would be useful based on the particular characteristics of the drugs and the disease involved.

In one embodiment, the treatment method includes the co-administration of a compound as disclosed herein or a pharmaceutically acceptable salt thereof and at least one immunotherapy. Immunotherapy (also called biological response modifier therapy, biologic therapy, biotherapy, immune therapy, or biological therapy) is a treatment that uses parts of the immune system to fight disease. Immunotherapy can help the immune system recognize cancer cells, or enhance a response against cancer cells. Immunotherapies include active and passive immunotherapies. Active immunotherapies stimulate the body's own immune system while passive immunotherapies generally use immune system components created outside of the body.

Examples of active immunotherapies include, but are not limited to vaccines including cancer vaccines, tumor cell vaccines (autologous or allogeneic), dendritic cell vaccines, antigen vaccines, anti-idiotype vaccines, DNA vaccines, viral vaccines, or Tumor-Infiltrating Lymphocyte (TIL) Vaccine with Interleukin-2 (IL-2) or Lymphokine-Activated Killer (LAK) Cell Therapy.

Examples of passive immunotherapies include but are not limited to monoclonal antibodies and targeted therapies containing toxins. Monoclonal antibodies include naked antibodies and conjugated monoclonal antibodies (also called tagged, labeled, or loaded antibodies). Naked monoclonal antibodies do not have a drug or radioactive material attached whereas conjugated monoclonal antibodies are joined to, for example, a chemotherapy drug (chemolabeled), a radioactive particle (radiolabeled), or a toxin (immunotoxin). Examples of these naked monoclonal antibody drugs include, but are not limited to Rituximab (Rituxan), an antibody against the CD20 antigen used to treat, for example, B cell non-Hodgkin lymphoma; Trastuzumab (Herceptin), an antibody against the HER2 protein used to treat, for example, advanced breast cancer; Alemtuzumab (Campath), an antibody against the CD52 antigen used to treat, for example, B cell chronic lymphocytic leukemia (B-CLL); Cetuximab (Erbitux), an antibody against the EGFR protein used, for example, in combination with irinotecan to treat, for example, advanced colorectal cancer and head and neck cancers; and Bevacizumab (Avastin) which is an antiangiogenesis therapy that works against the VEGF protein and is used, for example, in combination with chemotherapy to treat, for example, metastatic colorectal cancer. Examples of the conjugated monoclonal antibodies include, but are not limited to Radiolabeled antibody Ibritumomab tiuxetan (Zevalin) which delivers radioactivity directly to cancerous B lymphocytes and is used to treat, for example, B cell non-Hodgkin lymphoma; radiolabeled antibody Tositumomab (Bexxar) which is used to treat, for example, certain types of non-Hodgkin lymphoma; and immunotoxin Gemtuzumab ozogamicin (Mylotarg) which contains calicheamicin and is used to treat, for example, acute myelogenous leukemia (AML). BL22 is a conjugated monoclonal antibody for treating, for example, hairy cell leukemia, immunotoxins for treating, for example, leukemias, lymphomas, and brain tumors, and radiolabeled antibodies such as OncoScint for example, for colorectal and ovarian cancers and ProstaScint for example, for prostate cancers.

Further examples of therapeutic antibodies that can be used include, but are not limited to, HERCEPTIN™ (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3epitope) IgG antibody (ImClone System); IMC-C225 which is a chimeric anti-EGFR IgG antibody (ImClone System); VITAXIN™ which is a humanized anti-alpha V beta 3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatized anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-alpha antibody (CAT/BASF); CDP870 is a humanized anti-TNF-alpha. Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-alpha. IgG4 antibody (Celltech); LDP-02 is a humanized anti-alpha4 beta7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-beta₂ antibody (Cambridge Ab Tech). Others are provided in later paragraphs.

Immunotherapies that can be used in combination with a compound as disclosed herein include adjuvant immunotherapies. Examples include cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guerin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of, interleukins, for example, IL-2 with other cytokines, such as IFN-alpha.

In various embodiments, a compound of the present invention can be combined with an immunological therapy and/or an immunological therapeutic agent. In various embodiments, an immunological therapy and/or an immunological therapeutic agent can include, one or more of the following: an adoptive cell transfer, an angiogenesis inhibitor, Bacillus Calmette-Guerin therapy, biochemotherapy, a cancer vaccine, a chimeric antigen receptor (CAR) T-cell therapy, a cytokine therapy, gene therapy, an immune checkpoint modulator, an immunoconjugate, a radioconjugate, an oncolytic virus therapy, or a targeted drug therapy. The immunological therapy or immunological therapeutic agent, is collectively referred to herein as an “immunotherapeutic agent”.

The present disclosure provides a method for preventing, treating, reducing, inhibiting or controlling a neoplasia, a tumor or a cancer in a subject in need thereof, involving administering a therapeutically effective amount of a combination comprising a compound of the invention and an immunotherapeutic agent. In one non-limiting embodiment, the method comprises administering a therapeutically effective amount of a combination comprising a compound of the invention in combination with an immunotherapeutic agent. In various embodiments, the combination provides a cooperative effect, an additive effect, or a synergistic effect in reducing the number of cancer cells when treated with the combination as compared to each treatment alone. In some embodiments, administration of a therapeutically effective amount of a combination comprising a compound of the invention and an immunotherapeutic agent, results in synergistic anti-tumor activity and/or antitumor activity that is more potent than the additive effect of administration of a compound of the invention or immunotherapeutic agent alone.

Human cancers harbor numerous genetic and epigenetic alterations, generating neoantigens potentially recognizable by the immune system (Sjoblom et al. (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

The present disclosure provides a combination of a compound of the invention and an immunotherapeutic agent. These exemplified combinations can be used to treat a subject with a cancer. In various embodiments, immunotherapeutic agents that find utility in the present compositions, formulations, and methods can include one or more agents or therapies, including: an adoptive cell transfer, an angiogenesis inhibitor, Bacillus Calmette-Guerin therapy, biochemotherapy, a cancer vaccine, a chimeric antigen receptor (CAR) T-cell therapy, a cytokine therapy, gene therapy, an immune checkpoint modulator, for example an immune checkpoint inhibitor, an immunoconjugate, a radioconjugate, an oncolytic virus therapy, or a targeted drug therapy.

In certain embodiments of the present disclosure, a therapeutically effective combination comprises a compound of the invention and an immunotherapeutic agent. In various related embodiments, the compound of the invention enhances the activity of the immunotherapeutic agent.

In certain embodiments of each of the aforementioned aspects, as well as other aspects and embodiments described elsewhere herein, the immunotherapeutic agent enhances the activity of the compound of the invention.

In certain embodiments of each of the aforementioned aspects, as well as other aspects and embodiments described elsewhere herein, the compound of the invention and the immunotherapeutic agent act synergistically. In various embodiments described herein, an exemplary immunotherapeutic agent is an immune cell (e.g. T-cell, dendritic cell, a natural killer cell and the like) modulator chosen from an agonist or an activator of a costimulatory molecule, wherein the modulator is a monoclonal antibody, a bispecific antibody comprising one or more immune checkpoint antigen binding moieties, a trispecific antibody, or an immune cell-engaging multivalent antibody/fusion protein/construct known in the art. In some embodiments, the immunotherapeutic agent can be an antibody that modulates a costimulatory molecule, bind to an antigen on the surface of an immune cell, or a cancer cell.

In each of these different embodiments, the antibody modulator can be a monoclonal antibody, a polyclonal antibody, a bispecific antibody, a trispecific or multispecific format antibody, a fusion protein, or a fragment thereof, for example, a Diabody, a Single-chain (sc)-diabody (scFv)2, a Miniantibody, a Minibody, a Barnase-barstar, a scFv-Fc, a sc(Fab)2, a Trimeric antibody construct, a Triabody antibody construct, a Trimerbody antibody construct, a Tribody antibody construct, a Collabody antibody construct, a (scFv-TNFa)3, or a F(ab)3/DNL antibody construct.

In certain embodiments of each of the aforementioned aspects, as well as other aspects and embodiments described elsewhere herein, the immunotherapeutic agent is an agent that modulates immune responses, for example, a checkpoint inhibitor or a checkpoint agonist. In some embodiments, the immunotherapeutic agent is an agent that enhances anti-tumor immune responses. In some embodiments, the immunotherapeutic agent is an agent that increases cell-mediated immunity. In some embodiments, the immunotherapeutic agent is an agent that increases T-cell activity. In some embodiments, the immunotherapeutic agent is an agent that increases cytolytic T-cell (CTL) activity.

In some embodiments, the present methods of treatment may include administering a compound of the present invention together in combination with a molecule, for example, a binding agent, for example, an antibody of functional fragment thereof that modulates (activates or inhibits) a checkpoint protein. A checkpoint inhibitor can be any molecule, agent, treatment and/or method of inhibiting an immune checkpoint, and/or promoting an inhibitor of an immune checkpoint, e.g., by promoting an intrinsic immune checkpoint inhibitor; inhibiting a transcription factor involved in the expression of an immune checkpoint; and/or by acting in concert with some additional extrinsic factor. For example, a checkpoint inhibitor could include a treatment that inhibits transcription factors involved the expression of immune checkpoint genes, or promotes the expression of transcription factors for tumor-suppressor genes, e.g., BACH2 (Luan et al., (2016). Transcription Factors and Checkpoint Inhibitor Expression with Age: Markers of Immunosenescence. Blood, 128(22), 5983). Moreover, a checkpoint inhibitor can inhibit the transcription of immune checkpoint genes; the modification and/or processing of immune checkpoint mRNA; the translation of immune checkpoint proteins; and/or molecules involved in immunity or the immune checkpoint pathway, e.g., PD-1 transcription factors such as HIF-1, STAT3, NF-κB, and AP-1, or the activation of common oncogenic pathways such as JAK/STAT, RAS/ERK, or PI3K/AKT/mTOR (Zerdes et al., Genetic, transcriptional and post-translational regulation of the programmed death protein ligand 1 in cancer: biology and clinical correlations, Oncogene volume 37, pages 4639-4661 (2018), the disclosure of which is incorporated herein by reference in its entirety).

Checkpoint inhibitors can include treatments, molecules, agents, and/or methods that regulate immune checkpoints at the transcriptional level, e.g., using the RNA-interference pathway co-suppression, and/or post-transcriptional gene silencing (PTGS) (e.g., microRNAs, miRNA; silencing-RNA, small-interfering-RNA, or short-interfering-RNA (siRNA). Transcriptional regulation of checkpoint molecules has been shown to involve mir-16, which has been shown to target the 3′UTR of the checkpoint mRNAs CD80, CD274 (PD-L1) and CD40 (Leibowitz et al., Post-transcriptional regulation of immune checkpoint genes by mir-16 in melanoma, Annals of Oncology (2017) 28; v428-v448). Mir-33a has also been shown to be involved in regulating the expression of PD-1 in cases of lung adenocarcinoma (Boldini et al., Role of microRNA-33a in regulating the expression of PD-1 in lung adenocarcinoma, Cancer Cell Int. 2017; 17: 105, the disclosure of which is incorporated herein by reference in its entirety).

T-cell-specific aptamer-siRNA chimeras have been suggested as a highly specific method of inhibiting molecules in the immune checkpoint pathway (Hossain et al., The aptamer-siRNA conjugates: reprogramming T cells for cancer therapy, Ther. Deliv. 2015 January; 6(1): 1-4, the disclosure of which is incorporated herein by reference in its entirety).

Alternatively, members of the immune checkpoint pathway can be inhibited using treatments that affect associated pathways, e.g., metabolism. For example, oversupplying the glycolytic intermediate pyruvate in mitochondria from CAD macrophages promoted expression of PD-L1 via induction of the bone morphogenetic protein 4/phosphorylated SMAD1/5/IFN regulatory factor 1 (BMP4/p-SMAD1/5/IRF1) signaling pathway. Accordingly, implementing treatments that modulate the metabolic pathway can result in subsequent modulation of the immunoinhibitory PD-1/PD-L1 checkpoint pathway (Watanabe et al., Pyruvate controls the checkpoint inhibitor PD-L1 and suppresses T cell immunity, J Clin Invest. 2017 Jun. 30; 127(7): 2725-2738).

Checkpoint immunity can be regulated via oncolytic viruses that selectively replicate within tumor cells and induce acute immune responses in the tumor-micro-environment, i.e., by acting as genetic vectors that carry specific agents (e.g., antibodies, miRNA, siRNA, and the like) to cancer cells and effecting their oncolysis and secretion of cytokines and chemokines to synergize with immune checkpoint inhibition (Shi et al., Cancer Immunotherapy: A Focus on the Regulation of Immune Checkpoints, Int J Mol Sci. 2018 May; 19(5): 1389). Currently, there are clinical trials underway that utilize the following viruses as checkpoint inhibitors: poliovirus, measles virus, adenoviruses, poxviruses, herpes simplex virus (HSV), coxsackieviruses, reovirus, Newcastle disease virus (NDV), T-VEC (a herpes virus encoded with GM-CSF (granulocyte-macrophage colony stimulating factor)), and H101 (Shi et al., supra).

Checkpoint inhibitors can operate at the translational level of checkpoint immunity. The translation of mRNA into protein represents a key event in the regulation of gene expression, thus inhibition of immune checkpoint translation is a method in which the immune checkpoint pathway can be inhibited.

Inhibition of the immune checkpoint pathway can occur at any stage of the immune checkpoint translational process. For example, drugs, molecules, agents, treatments, and/or methods can inhibit the initiation process (whereby the 40S ribosomal subunit is recruited to the 5′ end of the mRNA and scans the 5′UTR of the mRNA toward its 3′ end. Inhibition can occur by targeting the anticodon of the initiator methionyl-transfer RNA (tRNA) (Met-tRNAi), its base-pairing with the start codon, or the recruitment of the 60S subunit to begin elongation and sequential addition of amino acids in the translation of immune-checkpoint-specific genes. Alternatively, a checkpoint inhibitor can inhibit checkpoints at the translational level by preventing the formation of the ternary complex (TC), i.e., eukaryotic initiation factor (eIF)2 (or one or more of its a, (3, and y subunits); GTP; and Met-tRNAi.

Checkpoint inhibition can occur via destabilization of eIF2a by precluding its phosphorylation via protein kinase R (PKR), PERK, GCN2, or HRI, or by precluding TCs from associating with the 40S ribosome and/or other initiation factors, thus preventing the preinitiation complex (PIC) from forming; inhibiting the eIF4F complex and/or its cap-binding protein eIF4E, the scaffolding protein eIF4G, or eIF4A helicase. Methods discussing the translational control of cancer are discussed in Truitt et al., New frontiers in translational control of the cancer genome, Nat Rev Cancer. 2016 Apr. 26; 16(5): 288-304, the disclosure of which is incorporated herein by reference in its entirety.

Checkpoint inhibitors can also include treatments, molecules, agents, and/or methods that regulate immune checkpoints at the cellular and/or protein level, e.g., by inhibiting an immune checkpoint receptor. Inhibition of checkpoints can occur via the use of antibodies, antibody fragments, antigen-binding fragments, small-molecules, and/or other drugs, agents, treatments, and/or methods.

Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response (‘block’ the immune response) against tumor tissues. In contrast to the majority of anti-cancer agents, checkpoint inhibitors do not target tumor cells directly, but rather target lymphocyte receptors or their ligands in order to enhance the endogenous antitumor activity of the immune system. (Pardoll, 2012, Nature Reviews Cancer 12:252-264).

In some embodiments, the immunotherapeutic agent is a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, or an immunostimulatory oligonucleotide. In some embodiments, the immune checkpoint modulator, i.e. is an inhibitor or antagonist, or is an activator or agonist, for example, a CD28 modulator, a 4-1BB modulator, an OX40 modulator, a CD27 modulator, a CD80 modulator, a CD86 modulator, a CD40 modulator, or a GITR modulator, a Lag-3 modulator, a 41BB modulator, a LIGHT modulator, a CD40 modulator, a GITR modulator, a TGF-beta modulator, a TIM-3 modulator, a SIRP-alpha modulator, a TIGIT modulator, a VSIG8 modulator, a BTLA modulator, a SIGLEC7 modulator, a SIGLEC9 modulator, a ICOS modulator, a B7H3 modulator, a B7H4 modulator, a FAS modulator, and/or a BTNL2 modulator. In some embodiments, the immunotherapeutic agent is an immune checkpoint modulator as described above (e.g., an immune checkpoint modulator antibody, which can be in the form of a monoclonal antibody, a bispecific antibody comprising one or more immune checkpoint antigen binding moieties, a trispecific antibody, or an immune cell-engaging multivalent antibody/fusion protein/construct known in the art).

In some embodiments, the immunotherapeutic agent is an agent that inhibits the activity of PD-1. In some embodiments, the immunotherapeutic agent is an agent that inhibits the activity of PD-L1 and/or PD-L2. In some embodiments, the immunotherapeutic agent is an agent that inhibits the activity of CTLA-4. In some embodiments, the immunotherapeutic agent is an agent that inhibits the activity of CD80 and/or CD86. In some embodiments, the immunotherapeutic agent is an agent that inhibits the activity of TIGIT. In some embodiments, the immunotherapeutic agent is an agent that inhibits the activity of KIR. In some embodiments, the immunotherapeutic agent is an agent that enhances or stimulates the activity of activating immune checkpoint receptors.

PD-1 (also known as Programmed Death 1, CD279, PDCD1) is a cell surface receptor with a critical role in regulating the balance between stimulatory and inhibitory signals in the immune system and maintaining peripheral tolerance (Ishida, Y et al. 1992 EMBO J. 11 3887; Kier, Mary E et al. 2008 Annu Rev Immunol 26 677-704; Okazaki, Taku et al. 2007 International Immunology 19 813-824). PD-1 is an inhibitory member of the immunoglobulin super-family with homology to CD28. The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Expression of PD-1 is inducible on T cells, B cells, natural killer (NK) cells and monocytes, for example upon lymphocyte activation via T cell receptor (TCR) or B cell receptor (BCR) signalling (Kier, Mary E et al. 2008 Annu Rev Immunol 26 677-704; Agata, Y et al 1996 Int Immunol 8 765-72). PD-1 is a receptor for the ligands CD80, CD86, PD-L1 (B7-H1, CD274) and PD-L2 (B7-DC, CD273), which are cell surface expressed members of the B7 family (Freeman, Gordon et al. 2000 J Exp Med 192 1027; Latchman, Y et al. 2001 Nat Immunol 2 261). Upon ligand engagement, PD-1 recruits phosphatases such as SHP-1 and SHP-2 to its intracellular tyrosine motifs which subsequently dephosphorylate effector molecules activated by TCR or BCR signalling (Chemnitz, J et al. 2004 J Immunol 173 945-954; Riley, James L 2009 Immunological Reviews 229 114-125) In this way, PD-1 transduces inhibitory signals into T and B cells only when it is engaged simultaneously with the TCR or BCR.

PD-1 has been demonstrated to down-regulate effector T cell responses via both cell-intrinsic and cell-extrinsic functional mechanisms. Inhibitory signaling through PD-1 induces a state of unresponsiveness in T cells, resulting in the cells being unable to clonally expand or produce optimal levels of effector cytokines. PD-1 may also induce apoptosis in T cells via its ability to inhibit survival signals from co-stimulation, which leads to reduced expression of key anti-apoptotic molecules such as Bcl-XL (Kier, Mary E et al. 2008 Annu Rev Immunol 26 677-704). In addition to these direct effects, recent publications have implicated PD-1 as being involved in the suppression of effector cells by promoting the induction and maintenance of regulatory T cells (TREG). For example, PD-L1 expressed on dendritic cells was shown to act in synergy with TGF-β to promote the induction of CD4+FoxP3+ TREG with enhanced suppressor function (Francisco, Loise M et al. 2009 J Exp Med 206 3015-3029).

TIM-3 (also known as T-cell immunoglobulin and mucin-domain containing-3, TIM-3, Hepatitis A virus cellular receptor 2, HAVCR2, HAVcr-2, KIM-3, TIMD-3, TIMD3, Tim-3, and CD366) is a ˜33.4-kDa single-pass type I membrane protein involved in immune responses (Sanchez-Fueyo et al., Tim-3 inhibits T helper type 1-mediated auto- and alloimmune responses and promotes immunological tolerance, Nat. Immunol. 4:1093-1101(2003)).

TIM-3 is selectively expressed on Th1-cells, and phagocytic cells (e.g., macrophages and dendritic cells). The use of siRNA or a blocking antibody to reduce the expression of human resulted in increased secretion of interferon γ (IFN-γ) from CD4 positive T-cells, implicating the inhibitory role of TIM-3 in human T cells. Analysis of clinical samples from autoimmune disease patients showed no expression of TIM-3 in CD4 positive cells. In particular, expression level of TIM-3 is lower and secretion of IFN-γ is higher in T cell clones derived from the cerebrospinal fluid of patients with multiple sclerosis than those in clones derived from normal healthy persons (Koguchi K et al., J Exp Med. 203:1413-8. (2006)).

TIM-3 is the receptor for the ligands Galectin-9, which is a member of galectin family, molecules ubiquitously expressed on a variety of cell types and which binds β-galactoside; Phospatidyl serine (PtdSer) (DeKryff et al., T cell/transmembrane, Ig, and mucin-3 allelic variants differentially recognize phosphatidylserine and mediate phagocytosis of apoptotic cells, J Immunol. 2010 Feb. 15; 184(4): 1918-30); High Mobility Group Protein 1 (also known as HMGB1, HMG1, HMG3, SBP-1, HMG-1, and high mobility group box 1) Chiba et al., Tumor-infiltrating DCs suppress nucleic acid-mediated innate immune responses through interactions between the receptor TIM-3 and the alarmin HMGB1, Nat Immunol. 2012 September; 13(9):832-42); and Carcinoembryonic Antigen Related Cell Adhesion Molecule 1 (also known as CEACAM1, BGP, BGP1, BGPI, carcinoembryonic antigen related cell adhesion molecule 1) (Huang et al., CEACAM1 regulates TIM-3-mediated tolerance and exhaustion, Nature. 2015 Jan. 15; 517(7534):386-90).

BTLA (also known as B- and T-lymphocyte attenuator, BTLA1, CD272, and B and T lymphocyte associated) is a ˜27.3-kDa single-pass type I membrane protein involved in lymphocyte inhibition during immune response. BTLA is constitutively expressed in both B and T cells. BTLA interacts with HVEM (herpes virus-entry mediator), a member of the tumor-necrosis factor receptor (TNFR) family (Gonzalez et al., Proc. Natl. Acad. Sci. USA, 2005, 102: 1116-21). The interaction of BTLA, which belongs to the CD28 family of the immunoglobulin superfamily, and HVEM, a costimulatory tumor-necrosis factor (TNF) receptor (TNFR), is unique in that it defines a cross talk between these two families of receptors. BTLA contains a membrane proximal immunoreceptor tyrosine-based inhibitory motif (ITIM) and membrane distal immunoreceptor tyrosine-based switch motif (ITSM). Disruption of either the ITIM or ITSM abrogated the ability of BTLA to recruit either SHP1 or SHP2, suggesting that BTLA recruits SHP1 and SHP2 in a manner distinct from PD-1 and both tyrosine motifs are required to block T cell activation. The BTLA cytoplasmic tail also contains a third conserved tyrosine-containing motif within the cytoplasmic domain, similar in sequence to a Grb-2 recruitment site (YXN). Also, a phosphorylated peptide containing this BTLA N-terminal tyrosine motif can interact with GRB2 and the p85 subunit of PI3K in vitro, although the functional effects of this interaction remain unexplored in vivo (Gavrieli et al., Bioochem. Biophysi Res Commun, 2003, 312, 1236-43). BTLA is the receptor for the ligands PTPN6/SHP-1; PTPN11/SHP-2; TNFRSF14/HVEM; and B7H4.

VISTA (also known as V-domain Ig suppressor of T cell activation VSIR, B7-H5, B7H5, GI24, PP2135, SISP1, DD1alpha, VISTA, C10orf54, chromosome 10 open reading frame 54, PD-1H, and V-set immunoregulatory receptor) is a ˜33.9-kDa single-pass type I membrane protein involved in T-cell inhibitory response, embryonic stem cells differentiation via BMP4 signaling inhibition, and MMP14-mediated MMP2 activation (Yoon et al., Control of signaling-mediated clearance of apoptotic cells by the tumor suppressor p53, Science. 2015 Jul. 31; 349(6247): 1261669). VISTA interacts with the ligand VSIG-3 (Wang et al., VSIG-3 as a ligand of VISTA inhibits human T-cell function, Immunology. 2019 January; 156(1):74-85)

LAG-3 (also known as Lymphocyte-activation gene 3, LAG3, CD223, and lymphocyte activating 3) is a ˜57.4-kDa single-pass type I membrane protein involved in lymphocyte activation that also binds to HLA class-II antigens. LAG-3 is a member of the immunoglobulin supergene family, and is expressed on activated T cells (Huard et al., 1994, Immunogenetics 39:213), NK cells (Triebel et al., 1990, J. Exp. Med. 171:1393-1405), regulatory T cells (Huang et al., 2004, Immunity 21:503-513; Camisaschi et al., 2010, J Immunol. 184:6545-6551; Gagliani et al., 2013, Nat Med 19:739-746), and plasmacytoid dendritic cells (DCs) (Workman et al., 2009, J Immunol 182:1885-1891). LAG-3 is a membrane protein encoded by a gene located on chromosome 12, and is structurally and genetically related to CD4. Similar to CD4, LAG-3 can interact with MHC class II molecules on the cell surface (Baixeras et al., 1992, J. Exp. Med. 176:327-337; Huard et al., 1996, Eur. J. Immunol. 26:1180-1186). It has been suggested that the direct binding of LAG-3 to MHC class II plays a role in down-regulating antigen-dependent stimulation of CD4+ T lymphocytes (Huard et al., 1994, Eur. J. Immunol. 24:3216-3221) and LAG-3 blockade has also been shown to reinvigorate CD8+ lymphocytes in both tumor or self-antigen (Gross et al., 2007, J Clin Invest. 117:3383-3392) and viral models (Blackburn et al., 2009, Nat. Immunol. 10:29-37). Further, the intra-cytoplasmic region of LAG-3 can interact with LAP (LAG-3-associated protein), which is a signal transduction molecule involved in the downregulation of the CD3/TCR activation pathway (Iouzalen et al., 2001, Eur. J. Immunol. 31:2885-2891). Moreover, CD4+CD25+ regulatory T cells (Treg) have been shown to express LAG-3 upon activation, which contributes to the suppressor activity of Treg cells (Huang, C. et al., 2004, Immunity 21:503-513). LAG-3 can also negatively regulate T cell homeostasis by Treg cells in both T cell-dependent and independent mechanisms (Workman, C. J. and Vignali, D. A., 2005, J. Immunol. 174:688-695).

LAG-3 has been shown to interact with MHC class II molecules (Huard et al., CD4/major histocompatibility complex class II interaction analyzed with CD4- and lymphocyte activation gene-3 (LAG-3)-Ig fusion proteins, Eur J Immunol. 1995 September; 25(9):2718-21).

Additionally, several kinases are known to be checkpoint inhibitors. For example, CHEK-1, CHEK-2, and A2aR.

CHEK-1 (also known as CHK 1 kinase, CHK1, and checkpoint kinase 1) is a ˜54.4-kDa serine/threonine-protein kinase that is involved with checkpoint-mediated cell cycle arrest, and the activation of DNA repair in response to the DNA damage and/or unreplicated DNA.

CHEK-2 (also known as CHK2 kinase, CDS1, CHK2, HuCds1, LFS2, PP1425, RAD53, hCds1, and checkpoint kinase 2) is a ˜60.9-kDa. serine/threonine-protein kinase involved in checkpoint-mediated cell cycle arrest, DNA-repair activation, and double-strand break-mediated apoptosis.

A2aR (also known as adenosine A2A receptor, ADORA2A, adenosine A2a receptor, A2aR, ADORA2, and RDC8) is a ˜44.7-kDa multi-pass membrane receptor for adenosine and other ligands.

In some embodiments, illustrative immunotherapeutic agents can include one or more antibody modulators that target PD-1, PD-L1, PD-L2, CEACAM (e.g., CEACAM-1, -3 and/or -5), CTLA-4, TIM-3, LAG-3, VISTA, BTLA, TIGIT, LAIR1, CD 160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, and/or BTNL2 among others known in the art. In some embodiments, the immunotherapeutic agent is an agent that increases natural killer (NK) cell activity. In some embodiments, the immunotherapeutic agent is an agent that inhibits suppression of an immune response. In some embodiments, the immunotherapeutic agent is an agent that inhibits suppressor cells or suppressor cell activity. In some embodiments, the immunotherapeutic agent is an agent or therapy that inhibits Treg activity. In some embodiments, the immunotherapeutic agent is an agent that inhibits the activity of inhibitory immune checkpoint receptors.

In some embodiments, the combination of the present disclosure comprises a compound of the invention and an immunotherapeutic agent, wherein the immunotherapeutic agent includes a T cell modulator chosen from an agonist or an activator of a costimulatory molecule. In one embodiment, the agonist of the costimulatory molecule is chosen from an agonist (e.g., an agonistic antibody or antigen-binding fragment thereof, or a soluble fusion) of GITR, OX40, SLAM (e.g., SLAMF7), HVEM, LIGHT, CD2, CD27, CD28, CDS, ICAM-1, LFA-1 (CD11a/CD18), ICOS (CD278), 4-1BB (CD137), CD30, CD40, BAFFR, CD7, NKG2C, NKp80, CD160, B7-H3, or CD83 ligand. In other embodiments, the effector cell combination includes a bispecific T cell engager (e.g., a bispecific antibody molecule that binds to CD3 and a tumor antigen (e.g., EGFR, PSCA, PSMA, EpCAM, HER2 among others).

In some embodiments, the immunotherapeutic agent is a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a modulator of SIRP-alpha activity, a modulator of TIGIT activity, a modulator of VSIG8 activity, a modulator of BTLA activity, a modulator of SIGLEC7 activity, a modulator of SIGLEC9 activity, a modulator of ICOS activity, a modulator of B7H3 activity, a modulator of B7H4 activity, a modulator of FAS activity, a modulator of BTNL2 activity, a cytokine, a chemokine, an interferon, an interleukin, a lymphokine, a member of the tumor necrosis factor (TNF) family, or an immunostimulatory oligonucleotide.

In some embodiments, the immunotherapeutic agent is an immune checkpoint modulator (e.g., an immune checkpoint inhibitor e.g. an inhibitor of PD-1 activity, a modulator of PD-L1 activity, a modulator of PD-L2 activity, a modulator of CTLA-4, or a CD40 agonist (e.g., an anti-CD40 antibody molecule), (xi) an OX40 agonist (e.g., an anti-OX40 antibody molecule), or (xii) a CD27 agonist (e.g., an anti-CD27 antibody molecule). In one embodiment, the immunotherapeutic agent is an inhibitor of: PD-1, PD-L1, PD-L2, CTLA-4, TIM-3, LAG-3, CEACAM (e.g., CEACAM-1, -3 and/or -5), VISTA, BTLA, TIGIT, LAIR1, CD 160, 2B4 and/or TGF beta, Galectin 9, CD69, Galectin-1, CD113, GPR56, CD48, GARP, PD1H, LAIR1, TIM-1, and TIM-4. In one embodiment, the inhibitor of an immune checkpoint molecule inhibits PD-1, PD-L1, LAG-3, TIM-3, CEACAM (e.g., CEACAM-1, -3 and/or -5), CTLA-4, or any combination thereof.

In one embodiment, the immunotherapeutic agent is an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, the immunotherapeutic agent used in the combinations disclosed herein (e.g., in combination with a compound of the invention) is an activator or agonist of a costimulatory molecule. In one embodiment, the agonist of the costimulatory molecule is chosen from an agonist (e.g., an agonistic antibody or antigen-binding fragment thereof, or a soluble fusion) of CD2, CD28, CDS, ICAM-1, LFA-1 (CD11a/CD18), ICOS (CD278), 4-1BB (CD137), GITR, CD30, BAFFR, HVEM, CD7, LIGHT, NKG2C, SLAMF7, NKp80, CD 160, B7-H3, or CD83 ligand.

Inhibition of an inhibitory molecule can be performed at the DNA, RNA or protein level. In embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody or antigen-binding fragment thereof, for example, a monoclonal antibody, a bispecific antibody comprising one or more immune checkpoint antigen binding moieties, a trispecific antibody, or an immune cell-engaging multivalent antibody/fusion protein/construct known in the art that binds to the inhibitory molecule; e.g., an antibody or fragment thereof (also referred to herein as “an antibody molecule”) that binds to PD-1, PD-L1, PD-L2, CTLA-4, TIM-3, LAG-3, CEACAM (e.g., CEACAM-1, -3 and/or -5), VISTA, BTLA, TIGIT, LAIR1, CD160, 2B4 and/or TGF beta, Galectin 9, CD69, Galectin-1, CD113, GPR56, CD48, GARP, PD1H, LAIR1, TIM-1, TIM-4, or a combination thereof.

In some embodiments, where the combination comprises a compound of the invention and an immunotherapeutic agent, wherein the immunotherapeutic agent is a monoclonal antibody or a bispecific antibody. For example, the monoclonal or bispecific antibody may specifically bind a member of the c-Met pathway and/or an immune checkpoint modulator (e.g., the bispecific antibody binds to both a hepatocyte growth factor receptor (HGFR) and an immune checkpoint modulator described herein, such as an antibody that binds PD-1, PD-L1, PD-L2, or CTLA-4, LAG-3, OX40, 41BB, LIGHT, CD40, GITR, TGF-beta, TIM-3, SIRP-alpha, TIGIT, VSIG8, BTLA, SIGLEC7, SIGLEC9, ICOS, B7H3, B7H4, FAS, BTNL2 or CD27). In particular embodiments, the bispecific antibody specifically binds a human HGFR protein and one of PD-1, PD-L1, and CTLA-4.

In some of the embodiments of the methods described herein, the immunotherapeutic agent is a PD-1 antagonist, a PD-L1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, or an IDO1 antagonist.

In some embodiments, the PD-1 antagonist is an antibody that specifically binds PD-1. In some embodiments, the antibody that binds PD-1 is pembrolizumab (KEYTRUDA®, MK-3475; Merck), pidilizumab (CT-011; Curetech Ltd.), nivolumab (OPDIVO®, BMS-936558, MDX-1106; Bristol Myer Squibb), MEDI0680 (AMP-514; AstraZenenca/MedImmune), REGN2810 (Regeneron Pharmaceuticals), BGB-A317 (BeiGene Ltd.), PDR-001 (Novartis), or STI-A1110 (Sorrento Therapeutics). In some embodiments, the antibody that binds PD-1 is described in PCT Publication WO 2014/179664, for example, an antibody identified as APE2058, APE1922, APE1923, APE1924, APE 1950, or APE1963 (Anaptysbio), or an antibody containing the CDR regions of any of these antibodies. In other embodiments, the PD-1 antagonist is a fusion protein that includes the extracellular domain of PD-L1 or PD-L2, for example, AMP-224 (AstraZeneca/MedImmune). In other embodiments, the PD-1 antagonist is a peptide inhibitor, for example, AUNP-12 (Aurigene).

In some embodiments, the PD-L1 antagonist is an antibody that specifically binds PD-L1. In some embodiments, the antibody that binds PD-L1 is atezolizumab (RG7446, MPDL3280A; Genentech), MEDI4736 (AstraZeneca/MedImmune), BMS-936559 (MDX-1105; Bristol Myers Squibb), avelumab (MSB0010718C; Merck KGaA), KD033 (Kadmon), the antibody portion of KD033, or STI-A1014 (Sorrento Therapeutics). In some embodiments, the antibody that binds PD-L1 is described in PCT Publication WO 2014/055897, for example, Ab-14, Ab-16, Ab-30, Ab-31, Ab-42, Ab-50, Ab-52, or Ab-55, or an antibody that contains the CDR regions of any of these antibodies, the disclosure of which is incorporated herein by reference in its entirety.

In some embodiments, the CTLA-4 antagonist is an antibody that specifically binds CTLA-4. In some embodiments, the antibody that binds CTLA-4 is ipilimumab (YERVOY®; Bristol Myer Squibb) or tremelimumab (CP-675,206; Pfizer). In some embodiments, the CTLA-4 antagonist a CTLA-4 fusion protein or soluble CTLA-4 receptor, for example, KARR-102 (Kahr Medical Ltd.).

In some embodiments, the LAG3 antagonist is an antibody that specifically binds LAG3. In some embodiments, the antibody that binds LAG3 is IMP701 (Prima BioMed), IMP731 (Prima BioMed/GlaxoSmithKline), BMS-986016 (Bristol Myer Squibb), LAG525 (Novartis), and GSK2831781 (GlaxoSmithKline). In some embodiments, the LAG3 antagonist includes a soluble LAG3 receptor, for example, IMP321 (Prima BioMed).

In some embodiments, the KIR antagonist is an antibody that specifically binds KIR. In some embodiments, the antibody that binds KIR is lirilumab (Bristol Myer Squibb/Innate Pharma).

In some embodiments, the immunotherapeutic agent is a cytokine, for example, a chemokine, an interferon, an interleukin, lymphokine, or a member of the tumor necrosis factor family. In some embodiments, the cytokine is IL-2, IL15, or interferon-gamma.

In some embodiments of any of the above aspects or those described elsewhere herein, the cancer is selected from the group consisting of lung cancer (e.g., a non-small cell lung cancer (NSCLC)), a kidney cancer (e.g., a kidney urothelial carcinoma), a bladder cancer (e.g., a bladder urothelial (transitional cell) carcinoma), a breast cancer, a colorectal cancer (e.g., a colon adenocarcinoma), an ovarian cancer, a pancreatic cancer, a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycoses fungoides, a merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor.

In some embodiments of any of the above aspects or those described elsewhere herein, the subject's cancer or tumor does not respond to immune checkpoint inhibition (e.g., to any immune checkpoint inhibitor described herein, such as a PD-1 antagonist or PD-L1 antagonist) or the subject's cancer or tumor has progressed following an initial response to immune checkpoint inhibition (e.g., to any immune checkpoint inhibitor described herein, such as a PD-1 antagonist or PD-L1 antagonist).

In various embodiments, the immunotherapeutic agent can comprise an antibody or an antigen binding fragment thereof. Within this definition, immune checkpoint inhibitors include bispecific antibodies and immune cell-engaging multivalent antibody/fusion protein/constructs known in the art. In some embodiments, immunotherapeutic agents which comprise bispecific antibodies may include bispecific antibodies that are bivalent and bind either the same epitope of the immune checkpoint molecule, two different epitopes of the same immune checkpoint molecule or different epitopes of two different immune checkpoints.

Persons of ordinary skill in the art can implement several bispecific antibody formats known in the field to target one or more of CTLA4, PD1, PD-L1 TIM-3, LAG-3, various B-7 ligands, B7H3, B7H4, CHK 1 and CHK2 kinases, BTLA, A2aR, OX40, 41BB, LIGHT, CD40, GITR, TGF-beta, SIRP-alpha, TIGIT, VSIG8, SIGLEC7, SIGLEC9, ICOS, FAS, BTNL2 and other for use in the combination described herein.

In various embodiments, the immunotherapeutic agent can include am immune cell-engaging multivalent antibody/fusion protein/construct.

In an embodiment of the disclosure, the checkpoint inhibitor, in combination with a compound of the invention, is used to reduce or inhibit metastasis of a primary tumor or cancer to other sites, or the formation or establishment of metastatic tumors or cancers at other sites distal from the primary tumor or cancer thereby inhibiting or reducing tumor or cancer relapse or tumor or cancer progression.

In a further embodiment of the disclosure, provided herein is a combination therapy for treating cancer, which comprises a compound of the invention and a checkpoint inhibitor with the potential to elicit potent and durable immune responses with enhanced therapeutic benefit and more manageable toxicity.

In a further embodiment of the disclosure, provided herein is a combination therapy for treating cancer, which comprises a compound of the invention and an immune checkpoint inhibitor. In an embodiment of the disclosure provided herein is a method for treating cancer and/or preventing the establishment of metastases by employing a compound of the present invention, which acts synergistically with a checkpoint inhibitor.

In further embodiments, the disclosure provides methods for one or more of the following: 1) reducing or inhibiting growth, proliferation, mobility or invasiveness of tumor or cancer cells that potentially or do develop metastases, 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established, 4) reducing or inhibiting formation or establishment of additional metastasis after the metastasis has been formed or established, 5) prolonged overall survival, 6) prolonged progression free survival, or 7) disease stabilization. The methods include administering to a subject in need thereof a compound of the present invention in combination with a checkpoint inhibitor as described herein.

In an embodiment of the disclosure, administration of a compound of the present invention with the immunotherapeutic agent provides a detectable or measurable improvement in a condition of a given subject, such as alleviating or ameliorating one or more adverse (physical) symptoms or consequences associated with the presence of a cell proliferative or cellular hyperproliferative disorder, neoplasia, tumor or cancer, or metastasis, i e., a therapeutic benefit or a beneficial effect.

A therapeutic benefit or beneficial effect is any objective or subjective, transient, temporary, or long-term improvement in the condition or pathology, or a reduction in onset, severity, duration or frequency of adverse symptom associated with or caused by cell proliferation or a cellular hyperproliferative disorder such as a neoplasia, tumor or cancer, or metastasis. It may lead to improved survival. A satisfactory clinical endpoint of a treatment method in accordance with the disclosure is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore may be, but is not limited to destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

Specific non-limiting examples of therapeutic benefit include a reduction in neoplasia, tumor or cancer, or metastasis volume (size or cell mass) or numbers of cells, inhibiting or preventing an increase in neoplasia, tumor or cancer volume (e.g., stabilizing), slowing or inhibiting neoplasia, tumor or cancer progression, worsening or metastasis, or inhibiting neoplasia, tumor or cancer proliferation, growth or metastasis.

In an embodiment of the disclosure, administration of the immunotherapeutic agent, in combination therapy with a compound of the invention, provides a detectable or measurable improvement or overall response according to the irRC (as derived from time-point response assessments and based on tumor burden), including one of more of the following: (i) irCR—complete disappearance of all lesions, whether measurable or not, and no new lesions (confirmation by a repeat, consecutive assessment no less than 4 weeks from the date first documented), (ii) irPR—decrease in tumor burden ≥50% relative to baseline (confirmed by a consecutive assessment at least 4 weeks after first documentation).

Optionally, any method described herein may not take effect immediately. For example, treatment may be followed by an increase in the neoplasia, tumor or cancer cell numbers or mass, but over time eventual stabilization or reduction in tumor cell mass, size or numbers of cells in a given subject may subsequently occur.

Additional adverse symptoms and complications associated with neoplasia, tumor, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of adverse symptom or complication associated with or caused by a cellular hyperproliferative disorder, an improvement in the subjects quality of life and/or well-being, such as increased energy, appetite, psychological well-being, are all particular non-limiting examples of therapeutic benefit.

A therapeutic benefit or improvement therefore can also include a subjective improvement in the quality of life of a treated subject. In additional embodiment, a method prolongs or extends lifespan (survival) of the subject. In a further embodiment, a method improves the quality of life of the subject.

In one embodiment, administration of the immunotherapeutic agent, in combination therapy with a compound of the invention, results in a clinically relevant improvement in one or more markers of disease status and progression selected from one or more of the following: (i): overall survival, (ii): progression-free survival, (iii): overall response rate, (iv): reduction in metastatic disease, (v): circulating levels of tumor antigens such as carbohydrate antigen 19.9 (CA19.9) and carcinembryonic antigen (CEA) or others depending on tumor, (vii) nutritional status (weight, appetite, serum albumin), (viii): pain control or analgesic use, (ix): CRP/albumin ratio.

Treatment with a compound of the invention in combination with an immunotherapeutic agent gives rise to more complex immunity including not only the development of innate immunity and type-1 immunity, but also immunoregulation which more efficiently restores appropriate immune functions.

In various exemplary methods, a checkpoint inhibitor antibody (monoclonal or polyclonal, bispecific, trispecific, or an immune cell-engaging multivalent antibody/fusion protein/construct) directed to a checkpoint molecule of interest (e.g., PD-1) may be sequenced and the polynucleotide sequence may then be cloned into a vector for expression or propagation. The sequence encoding the antibody or antigen-binding fragment thereof of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

Pharmaceutical compositions containing a compound of the invention according to the present disclosure will comprise an effective amount of a compound of the invention, an immunotherapeutic agent, and/or both, typically dispersed in a pharmaceutically acceptable carrier. The phrases “pharmaceutically or pharmacologically acceptable” refers to molecular entities and compositions that do not produce adverse, allergic or other untoward reaction when administered to animal, such as, for example, a human, as appropriate. The preparation of an pharmaceutical composition that contains a compound of the invention will be known to those of skill in the art in light of the present disclosure, as exemplified by Remington's Pharmaceutical Sciences, 18th Ed. Mack Printing Company, 1990, Moreover, for animal (e.g., human) administration, it will be understood that preparations should meet sterility, pyrogenicity, general safety and purity standards. A specific example of a pharmacologically acceptable carrier for a combination compositions, containing a compound of the invention in admixture with an immunotherapeutic agent as described herein is borate buffer or sterile saline solution (0.9% NaCl).

Formulations of the an immunotherapeutic agent, for example an immune checkpoint modulator antibody used in accordance with the present disclosure can be prepared for storage by mixing an antibody having the desired degree of purity with optional pharmaceutically acceptable carriers, excipients or stabilizers as amply described and illustrated in Remington's Pharmaceutical Sciences 16th edition, Osol, A. Ed. [1980], in the form of lyophilized formulations or aqueous solutions and/or suspensions. Acceptable carriers, excipients, buffers or stabilizers are nontoxic to recipients at the dosages and concentrations employed, and include suitable aqueous and/or non-aqueous excipients that may be employed in the pharmaceutical compositions of the disclosure, for example, water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants, buffers such as phosphate, citrate, and other organic acids. Antioxidants may be included, for example, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like; preservatives (such as octade-cyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues). Other exemplary pharmaceutically acceptable excipients may include polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In one illustrative embodiment, the pharmaceutical compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. In some embodiments, the checkpoint inhibitor antibodies or antigen-binding fragments thereof of the present disclosure are formulated for and can be lyophilized for storage and reconstituted in a suitable excipient prior to use according to art-known lyophilization and reconstitution techniques. In one exemplary pharmaceutical composition containing one or more checkpoint inhibitor antibodies or antigen-binding fragment thereof, the composition is formulated as a sterile, preservative-free solution of one or more checkpoint inhibitor antibodies or antigen-binding fragment thereof for intravenous or subcutaneous administration. The formulation can be supplied as either a single-use, prefilled pen, as a single-use, for example containing about 1 mL prefilled glass syringe, or as a single-use institutional use vial. Preferably, the pharmaceutical composition containing the checkpoint inhibitor antibody or antigen-binding fragment thereof is clear and colorless, with a pH of about 6.9-5.0, preferably a pH of 6.5-5.0, and even more preferably a pH ranging from about 6.0 to about 5.0. In various embodiments, the formulations comprising the pharmaceutical compositions can contain from about 500 mg to about 10 mg, or from about 400 mg to about 20 mg, or from about 300 mg to about 30 mg or from about 200 mg to about 50 mg of the checkpoint inhibitor antibody or antigen-binding fragment thereof per mL of solution when reconstituted and administered to the subject. Exemplary injection or infusion excipients can include mannitol, citric acid monohydrate, dibasic sodium phosphate dihydrate, monobasic sodium phosphate dihydrate, polysorbate 80, sodium chloride, sodium citrate and water for parenteral administration, for example, intravenously, intramuscularly, intraperitoneally, or subcutaneous administration.

In another exemplary embodiment, one or more immunotherapeutic agents, or an antigen-binding fragment thereof is formulated for intravenous or subcutaneous administration as a sterile aqueous solution containing 1-75 mg/mL, or more preferably, about 5-60 mg/mL, or yet more preferably, about 10-50 mg/mL, or even more preferably, about 10-40 mg/mL of antibody, with sodium acetate, polysorbate 80, and sodium chloride at a pH ranging from about 5 to 6. Preferably, the intravenous or subcutaneous formulation is a sterile aqueous solution containing 5, 10, 15, 20, 25, 30, 35, 40, 45, or 50 mg/mL of the immunotherapeutic agent, for example, an immune checkpoint inhibitor antibody or an antigen-binding fragment thereof, with 20 mM sodium acetate, 0.2 mg/mL polysorbate 80, and 140 mM sodium chloride at pH 5.5. Further, a solution comprising a checkpoint inhibitor antibody or an antigen-binding fragment thereof, can comprise, among many other compounds, histidine, mannitol, sucrose, trehalose, glycine, poly(ethylene)glycol, EDTA, methionine, and any combination thereof, and many other compounds known in the relevant art.

In one embodiment, a pharmaceutical composition of the present disclosure comprises the following components: 5-500 mg of an immunotherapeutic agent or antigen-binding fragment thereof of the present disclosure, 10 mM histidine, 5% sucrose, and 0.01% polysorbate 80 at pH 5.8, and a compound of the invention. This composition may be provided as a lyophilized powder. When the powder is reconstituted at full volume, the composition retains the same formulation. Alternatively, the powder may be reconstituted at half volume, in which case the composition comprises 10-500 mg of an immunotherapeutic agent or antigen-binding fragment thereof of the present disclosure, 20 mM histidine, 10% sucrose, and 0.02% polysorbate 80 at pH 5.8.

In one embodiment, part of the dose is administered by an intravenous bolus and the rest by infusion of the immunotherapeutic agent formulation. For example, from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the immunotherapeutic agent, or antigen-binding fragment thereof, may be given as a bolus, and the rest of the antibody dose may be administered by intravenous injection. A predetermined dose of the immunotherapeutic agent, or antigen-binding fragment thereof, may be administered, for example, over a period of an hour to two hours to five hours.

In a further embodiment, part of the dose is administered by a subcutaneous injection and/or infusion in the form of a bolus and the rest by infusion of the immunotherapeutic agent formulation. In some exemplary doses, the immunotherapeutic agent formulation can be administered subcutaneously in a dose ranging from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the immunotherapeutic agent, or antigen-binding fragment thereof. In some embodiments the dose may be given as a bolus, and the rest of the immunotherapeutic agent dose may be administered by subcutaneous or intravenous injection. A predetermined dose of the immunotherapeutic agent, or antigen-binding fragment thereof, may be administered, for example, over a period of an hour to two hours to five hours.

The formulation herein may also contain more than one active compound as necessary for the particular indication being treated, preferably those with complementary activities that do not adversely affect each other. For example, it may be desirable to provide one or more immunotherapeutic agents with other specificities. Alternatively, or in addition, the composition may comprise an anti-inflammatory agent, a chemotherapeutic agent, a cytotoxic agent, a cytokine, a growth inhibitory agent and/or a small molecule antagonist. Such molecules are suitably present in combination in amounts that are effective for the purpose intended.

The formulations to be used for in vivo administration should be sterile, or nearly so. This is readily accomplished by filtration through sterile filtration membranes.

In various embodiments, illustrative formulations of the pharmaceutical compositions described herein can be prepared using methods widely known in the field of pharmaceutical formulations. In general, such preparatory methods can include the step of bringing the active ingredient into association with a carrier or one or more other accessory ingredients, and then, if desirable, packaging the product into a desired single- or multi-dose unit.

In some embodiments, the composition comprising a compound of the invention can be also delivered in a vesicle, and the immunotherapeutic agent can be delivered in the same liposome formulation, or in a separate formulation that is compatible with the liposomal formulation containing the compound of the invention, In some illustrative examples, a liposome containing one or more liposomal surface moieties for example, polyethylene glycol, antibodies and antibody fragments thereof that target a desired tumor surface antigen, receptor, growth factor, glycoprotein, glycolipid or neoantigen, which are selectively transported into specific cells or organs, thus enhance targeted drug delivery.

In another embodiment, a compound of the invention can be delivered in a vesicle, in particular a liposome (see Langer, Science 249:1527-1533 (1990); Treat et al., in LIPOSOMES IN THE THERAPY OF INFECTIOUS DISEASE AND CANCER, Lopez-Berestein and Fidler (eds.), Liss, N.Y., pp. 353-365 (1989); Lopez-Berestein, ibid., pp. 317-327; see generally ibid.).

In yet another embodiment, a compound of the invention, or the composition containing the combination, or a composition containing the immunotherapeutic agent, can be delivered in a controlled release system. In one embodiment, a pump can be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, controlled release of the compound of the invention can comprise polymeric materials to provide sustained, intermediate, pulsatile, or alternate release (see MEDICAL APPLICATIONS OF CONTROLLED RELEASE, Langer and Wise (eds.), CRC Pres., Boca Raton, Fla. (1974); CONTROLLED DRUG BIOAVAILABILITY, DRUG PRODUCT DESIGN AND PERFORMANCE, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351(1989); Howard et al., J. Neurosurg. 71:105 (1989)). Other controlled-release systems discussed in the review by Langer (Science 249:1527-1533 (1990)) can be used.

The optimum concentration of the active ingredient(s) in the chosen medium can be determined empirically, according to procedures well known to the skilled artisan, and will depend on the ultimate pharmaceutical formulation desired and the use to be employed.

The present disclosure also provides a pharmaceutical pack or kit comprising one or more containers filled with one or more of the ingredients of the pharmaceutical compositions of the disclosure, which at minimum will include a compound of the invention and one or more checkpoint inhibitor antibodies or antigen-binding fragment thereof as described herein. In other embodiments, the kit may contain one or more further containers providing a pharmaceutically acceptable excipient, for example a diluent. In one embodiment a kit may comprise at least one container, wherein the container can include a compound of the invention, a checkpoint inhibitor antibody or an antigen-binding fragment thereof of the present disclosure. The kit may also include a set of instructions for preparing and administering the final pharmaceutical composition to the subject in need thereof, for the treatment of a checkpoint molecule-mediated disease or disorder.

Some embodiments of the present disclosure, the immunotherapeutic agent is a population of immune cells, which can be administered in combination with a compound of the invention to treat a subject with cancer. In some embodiments, the immunotherapeutic agent is a population of immune cells, such as leukocytes (nucleated white blood cells), comprising (e.g., expressing) a receptor that binds to an antigen of interest. A leukocyte of the present disclosure may be, for example, a neutrophil, eosinophil, basophil, lymphocyte or a monocyte. In some embodiments, a leukocyte is a lymphocyte. Examples of lymphocytes include T cells, B cells, Natural Killer (NK) cells or NKT cells. In some embodiments, a T-cell is a CD4+ Th (T helper) cell, a CD8+ cytotoxic T cell, a γδT cell or a regulatory (suppressor) T cell. In some embodiments, an immune cell is a dendritic cell.

Immune cells of the present disclosure, in some embodiments, are genetically engineered to express an antigen-binding receptor. A cell is considered “engineered” if it contains an engineered (exogenous) nucleic acid. Engineered nucleic acids of the present disclosure may be introduced into a cell by any known (e.g., conventional) method. For example, an engineered nucleic acid may be introduced into a cell by electroporation (see, e.g., Heiser W. C. Transcription Factor Protocols: Methods in Molecular Biology™ 2000; 130: 117-134), chemical (e.g., calcium phosphate or lipid), transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), microinjection of purified DNA directly into the nucleus of the cell (see, e.g., Capecchi M. R. Cell. 1980 November; 22(2 Pt 2): 479-88), or retrovirus transduction.

Some aspects of the present disclosure provide an “adoptive cell” approach, which involves isolating immune cells (e.g., T-cells) from a subject with cancer, genetically engineering the immune cells (e.g., to express an antigen-binding receptor, such as a chimeric antigen receptor), expanding the cells ex vivo, and then re-introducing the immune cells into the subject. This method results in a greater number of engineered immune cells in the subject relative to what could be achieved by conventional gene delivery and vaccination methods. In some embodiments, immune cells are isolated from a subject, expanded ex vivo without genetic modification, and then re-introduced into the subject.

Immune cells of the present disclosure comprise receptors that bind to antigens, such as an antigen encoded by an exogenously delivered nucleic acid, as provided herein. In some embodiments, a leukocyte is modified (e.g., genetically modified) to express a receptor that binds to an antigen. The receptor may be, in some embodiments, a naturally-occurring antigen receptor (normally expressed on the immune cell), recombinant antigen receptor (not normally expressed on the immune cell) or a chimeric antigen receptor (CAR). Naturally-occurring and recombinant antigen receptors encompassed by the present disclosure include T cell receptors, B cell receptors, NK cell receptors, NKT cell receptors and dendritic cell receptors. A “chimeric antigen receptor” refers to an artificial immune cell receptor that is engineered to recognize and bind to an antigen expressed by tumor cells. Generally, a CAR is designed for a T cell and is a chimera of a signaling domain of the T-cell receptor (TcR) complex and an antigen-recognizing domain (e.g., a single chain fragment (scFv) of an antibody) (Enblad et al., Human Gene Therapy. 2015; 26(8):498-505), the disclosure of which is incorporated herein by reference in its entirety.

In some embodiments, an antigen binding receptor is a chimeric antigen receptor (CAR). A T cell that expressed a CAR is referred to as a “CAR T cell.” A CAR T cell receptor, in some embodiments, comprises a signaling domain of the T-cell receptor (TcR) complex and an antigen-recognizing domain (e.g., a single chain fragment (scFv) of an antibody) (Enblad et al., Human Gene Therapy. 2015; 26(8):498-505) the disclosure of which is incorporated herein by reference in its entirety.

There are four generations of CARs, each of which contains different components. First generation CARs join an antibody-derived scFv to the CD3zeta (zeta. or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TcR CD3-zeta chain. Third-generation costimulatory domains may include, e.g., a combination of CD3z, CD27, CD28, 4-1BB, ICOS, or OX40. CARs, in some embodiments, contain an ectodomain (e.g., CD3), commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125(26):4017-4023; Kakarla and Gottschalk, Cancer J. 2014; 20(2): 151-155) the disclosure of which is incorporated herein by reference in its entirety.

In some embodiments, the chimeric antigen receptor (CAR) is a T-cell redirected for universal cytokine killing (TRUCK), also known as a fourth generation CAR. TRUCKS are CAR-redirected T-cells used as vehicles to produce and release a transgenic cytokine that accumulates in the targeted tissue, e.g., a targeted tumor tissue. The transgenic cytokine is released upon CAR engagement of the target. TRUCK cells may deposit a variety of therapeutic cytokines in the target. This may result in therapeutic concentrations at the targeted site and avoid systemic toxicity.

CARs typically differ in their functional properties. The CD3zeta signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-IBB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency. Fourth generation CARs are additionally modified with a constitutive or inducible expression cassette for a transgenic cytokine, which is released by the CAR T-cell to modulate the T-cell response. See, for example, Enblad et al., Human Gene Therapy. 2015; 26(8):498-505; Chmielewski and Hinrich, Expert Opinion on Biological Therapy. 2015; 15(8): 1145-1154 the disclosures of which are incorporated herein by reference in their entireties.

In some embodiments, an illustrative immunotherapeutic agent is a first generation chimeric antigen receptor CAR. In some embodiments, a chimeric antigen receptor is a third generation CAR. In some embodiments, a chimeric antigen receptor is a second generation CAR. In some embodiments, a chimeric antigen receptor is a third generation CAR. In some embodiments, the chimeric antigen receptor is a fourth generation CAR or a T-cell redirected for universal cytokine killing (TRUCK).

In some embodiments, a chimeric antigen receptor (CAR) comprises an extracellular domain comprising an antigen binding domain, a transmembrane domain, and a cytoplasmic domain. In some embodiments, a CAR is fully human. In some embodiments, the antigen binding domain of a CAR is specific for one or more antigens. In some embodiments, a “spacer” domain or “hinge” domain is located between an extracellular domain (comprising the antigen binding domain) and a transmembrane domain of a CAR, or between a cytoplasmic domain and a transmembrane domain of the CAR. A “spacer domain” refers to any oligopeptide or polypeptide that functions to link the transmembrane domain to the extracellular domain and/or the cytoplasmic domain in the polypeptide chain. A “hinge domain” refers to any oligopeptide or polypeptide that functions to provide flexibility to the CAR, or domains thereof, or to prevent steric hindrance of the CAR, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of a CAR.

In some embodiments, a CAR of the disclosure comprises an antigen binding domain, such as a single chain Fv (scFv) specific for a tumor antigen. The choice of binding domain depends upon the type and number of ligands that define the surface of a target cell. For example, the antigen binding domain may be chosen to recognize a ligand that acts as a cell surface marker on target cells associated with a particular disease state, such as cancer or an autoimmune disease. Thus, examples of cell surface markers that may act as ligands for the antigen binding domain in the CAR of the present disclosure include those associated with cancer cells and/or other forms of diseased cells. In some embodiments, a CAR is engineered to target a tumor antigen of interest by way of engineering a desired antigen binding domain that specifically binds to an antigen on a tumor cell encoded by an engineered nucleic acid, as provided herein.

An antigen binding domain (e.g., an scFv) that “specifically binds” to a target or an epitope is a term understood in the art, and methods to determine such specific binding are also known in the art. A molecule is said to exhibit “specific binding” if it reacts or associates more frequently, more rapidly, with greater duration and/or with greater affinity with a particular target antigen than it does with alternative targets. An antigen binding domain (e.g., an scFv) that specifically binds to a first target antigen may or may not specifically bind to a second target antigen. As such, “specific binding” does not necessarily require (although it can include) exclusive binding.

In some embodiments, immune cells expressing a CAR are genetically modified to recognize multiple targets or antigens, which permits the recognition of unique target or antigen expression patterns on tumor cells. Examples of CARs that can bind multiple targets include: “split signal CARs,” which limit complete immune cell activation to tumors expressing multiple antigens; “tandem CARs” (TanCARs), which contain ectodomains having two scFvs; and “universal ectodomain CARs,” which incorporate avidin or a fluorescein isothiocyanate (FITC)-specific scFv to recognize tumor cells that have been incubated with tagged monoclonal antibodies (Mabs).

A CAR is considered “bispecific” if it recognizes two distinct antigens (has two distinct antigen recognition domains). In some embodiments, a bispecific CAR is comprised of two distinct antigen recognition domains present in tandem on a single transgenic receptor (referred to as a TanCAR; see, e.g., Grada Z et al. Molecular Therapy Nucleic Acids 2013; 2:e105, incorporated herein by reference in its entirety). Thus, methods, in some embodiments, comprise delivering to a tumor a combination comprising a compound of the invention and an immunotherapeutic agent, wherein the immunotherapeutic agent is an engineered nucleic acid that encodes an antigen, or delivering to a tumor an engineered nucleic acid that induces expression of a self-antigen, and delivering to the tumor an immune cell expressing a bispecific CAR that binds to two antigens, one of which is encoded by the engineered nucleic acid.

In some embodiments, a CAR is an antigen-specific inhibitory CAR (iCAR), which may be used, for example, to avoid off-tumor toxicity (Fedorov, V D et al. Sci. Transl. Med. published online Dec. 11, 2013, incorporated herein by reference in its entirety). iCARs contain an antigen-specific inhibitory receptor, for example, to block nonspecific immunosuppression, which may result from extra tumor target expression. iCARs may be based, for example, on inhibitory molecules CTLA-4 or PD-1. In some embodiments, these iCARs block T cell responses from T cells activated by either their endogenous T cell receptor or an activating CAR. In some embodiments, this inhibiting effect is temporary.

In some embodiments, CARs may be used in adoptive cell transfer, wherein immune cells are removed from a subject and modified so that they express receptors specific to an antigen, e.g., a tumor-specific antigen. The modified immune cells, which may then recognize and kill the cancer cells, are reintroduced into the subject (Pule, et al., Cytotherapy. 2003; 5(3): 211-226; Maude et al., Blood. 2015; 125(26): 4017-4023, each of which is incorporated herein by reference in their entireties).

According to other aspects of the disclosure, the tumor antigenic component in the vaccine of the invention is any natural or synthetic tumor-associated protein or peptide or combination of tumor-associated proteins and/or peptides or glycoproteins or glycopeptides. In still yet other aspects, the antigenic component can be patient-specific or common to many or most patients with a particular type of cancer. According to one aspect, the antigenic component consists of a cell lysate derived from tumor tissue removed from the patient being treated. In another aspect, the lysate can be engineered or synthesized from exosomes derived from tumor tissue. In yet another aspect, the antigenic component consists of a cell lysate derived from tumor tissue extracted from one or more unrelated individuals or from tumor-cell lines.

In various embodiments, an illustrative immunotherapeutic agent comprises one or more cancer vaccines, for use in combination with a compound of the invention. The tumor-associated antigen component of the vaccine may be manufactured by any of a variety of well-known techniques. For individual protein components, the antigenic protein is isolated from tumor tissue or a tumor-cell line by standard chromatographic means such as high-pressure liquid chromatography or affinity chromatography or, alternatively, it is synthesized by standard recombinant DNA technology in a suitable expression system, such as E. coli, yeast or plants. The tumor-associated antigenic protein is then purified from the expression system by standard chromatographic means. In the case of peptide antigenic components, these are generally prepared by standard automated synthesis. Proteins and peptides can be modified by addition of amino acids, lipids and other agents to improve their incorporation into the delivery system of the vaccine (such as a multilamellar liposome). For a tumor-associated antigenic component derived from the patient's own tumor, or tumors from other individuals, or cell lines, the tumor tissue, or a single cell suspension derived from the tumor tissue, is typically homogenized in a suitable buffer. The homogenate can also be fractionated, such as by centrifugation, to isolate particular cellular components such as cell membranes or soluble material. The tumor material can be used directly or tumor-associated antigens can be extracted for incorporation in the vaccine using a buffer containing a low concentration of a suitable agent such as a detergent. An example of a suitable detergent for extracting antigenic proteins from tumor tissue, tumor cells, and tumor-cell membranes is diheptanoyl phosphatidylcholine. Exosomes derived from tumor tissue or tumor cells, whether autologous or heterologous to the patient, can be used for the antigenic component for incorporation in the vaccine or as a starting material for extraction of tumor-associated antigens.

In some embodiments of the present disclosure, a combination therapy comprises a compound of the present invention in combination with a cancer vaccine immunotherapeutic agent. In various examples, the cancer vaccine includes at least one tumor-associated antigen, at least one immunostimulant, and optionally, at least one cell-based immunotherapeutic agent. In some embodiments, the immunostimulant component in the cancer vaccine of the disclosure is any Biological Response Modifier (BRM) with the ability to enhance the therapeutic cancer vaccine's effectiveness to induce humoral and cellular immune responses against cancer cells in a patient. According to one aspect, the immunostimulant is a cytokine or combination of cytokines. Examples of such cytokines include the interferons, such as IFN-gamma, the interleukins, such as IL-2, IL-15 and IL-23, the colony stimulating factors, such as M-CSF and GM-CSF, and tumor necrosis factor.

According to another aspect, the immunostimulant component of the disclosed cancer vaccine includes one or more adjuvant-type immunostimulatory agents such as APC Toll-like Receptor agonists or costimulatory/cell adhesion membrane proteins, with or without immunostimulatory cytokines. Examples of Toll-like Receptor agonists include lipid A and CpG, and costimulatory/adhesion proteins such as CD80, CD86, and ICAM-1.

In some embodiments, the immunostimulant is selected from the group consisting of IFN-gamma (IFN-γ), IL-2, IL-15, IL-23, M-CSF, GM-CSF, tumor necrosis factor, lipid A, CpG, CD80, CD86, and ICAM-1, or combinations thereof. According to other aspects, the cell-based immunotherapeutic agent is selected from the group consisting of dendritic cells, tumor-infiltrating T lymphocytes, chimeric antigen receptor-modified T effector cells directed to the patient's tumor type, B lymphocytes, natural killer cells, bone marrow cells, and any other cell of a patient's immune system, or combinations thereof. In one aspect, the cancer vaccine immunostimulant includes one or more cytokines, such as interleukin 2 (IL-2), GM-CSF, M-CSF, and interferon-gamma (IFN-γ), one or more Toll-like Receptor agonists and/or adjuvants, such as monophosphoryl lipid A, lipid A, muramyl dipeptide (MDP) lipid conjugate and double stranded RNA, or one or more costimulatory membrane proteins and/or cell adhesion proteins, such CD80, CD86 and ICAM-1, or any combination of the above. In one aspect, the cancer vaccine includes an immunostimulant that is a cytokine selected from the group consisting of interleukin 2 (IL-2), GM-CSF, M-CSF, and interferon-gamma (IFN-γ). In another aspect, the cancer vaccine includes an immunostimulant that is a Toll-like Receptor agonist and/or adjuvant selected from the group consisting of monophosphoryl lipid A, lipid A, and muramyl dipeptide (MDP) lipid conjugate and double stranded RNA. In yet another aspect, the cancer vaccine includes an immunostimulant that is a costimulatory membrane protein and/or cell adhesion protein selected from the group consisting of CD80, CD86, and ICAM-1.

In various embodiments, an immunotherapeutic agent can include a cancer vaccine, wherein the cancer vaccine incorporates any tumor antigen that can be potentially used to construct a fusion protein according to the invention and particularly the following:

(a) cancer-testis antigens including NY-ESO-1, SSX2, SCP1 as well as RAGE, BAGE, GAGE and MAGE family polypeptides, for example, GAGE-1, GAGE-2, MAGE-1 MAGE-2, MAGE-3, MAGE-4, MAGE-5, MAGE-6, and MAGE-12, which can be used, for example, to address melanoma, lung, head and neck, NSCLC, breast, gastrointestinal, and bladder tumors; (b) mutated antigens, including p53, associated with various solid tumors, e.g., colorectal, lung, head and neck cancer; p21/Ras associated with, e.g., melanoma, pancreatic cancer and colorectal cancer; CDK4, associated with, e.g., melanoma; MUM1 associated with, e.g., melanoma; caspase-8 associated with, e.g., head and neck cancer; CIA 0205 associated with, e.g., bladder cancer; HLA-A2-R1701, beta catenin associated with, e.g., melanoma; TCR associated with, e.g., T-cell non-Hodgkin lymphoma; BCR-abl associated with, e.g., chronic myelogenous leukemia; triosephosphate isomerase; KIA 0205; CDC-27, and LDLR-FUT; (c) over-expressed antigens, including, Galectin 4 associated with, e.g., colorectal cancer; Galectin 9 associated with, e.g., Hodgkin's disease; proteinase 3 associated with, e.g., chronic myelogenous leukemia; WT 1 associated with, e.g., various leukemias; carbonic anhydrase associated with, e.g., renal cancer; aldolase A associated with, e.g., lung cancer; PRAME associated with, e.g., melanoma; HER-2/neu associated with, e.g., breast, colon, lung and ovarian cancer; mammaglobin, alpha-fetoprotein associated with, e.g., hepatoma; KSA associated with, e.g., colorectal cancer; gastrin associated with, e.g., pancreatic and gastric cancer; telomerase catalytic protein, MUC-1 associated with, e.g., breast and ovarian cancer; G-250 associated with, e.g., renal cell carcinoma; p53 associated with, e.g., breast, colon cancer; and carcinoembryonic antigen associated with, e.g., breast cancer, lung cancer, and cancers of the gastrointestinal tract such as colorectal cancer; (d) shared antigens, including melanoma-melanocyte differentiation antigens such as MART-1/Melan A; gp100; MC1R; melanocyte-stimulating hormone receptor; tyrosinase; tyrosinase related protein-1/TRP1 and tyrosinase related protein-2/TRP2 associated with, e.g., melanoma; (e) prostate associated antigens including PAP, PSA, PSMA, PSH-P1, PSM-P1, PSM-P2, associated with e.g., prostate cancer; (f) immunoglobulin idiotypes associated with myeloma and B cell lymphomas. In certain embodiments, the one or more TAA can be selected from pi 5, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, pl85erbB2, pi 80erbB-3, c-met, mn-23H1, TAG-72-4, CA 19-9, CA 72-4, CAM 17.1, NuMa, K-ras, pi 6, TAGE, PSCA, CT7, 43-9F, 5T4, 791 Tgp72, beta-HCG, BCA225, BTAA, CA 125, CA 15-3 (CA 27.29BCAA), CA 195, CA 242, CA-50, CAM43, CD68KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS or any combinations thereof.

In some embodiments, the present disclosure provides a compound of the present invention for use in combination with a cancer vaccine, which can include a tumor antigen comprising the entire amino acid sequence, a portion of it, or specific immunogenic epitopes of a human protein.

In various embodiments, an illustrative immunotherapeutic agent may include an mRNA operable to encode any one or more of the aforementioned cancer antigens useful for synthesizing a cancer vaccine. In some illustrative embodiments, the mRNA based cancer vaccine may have one or more of the following properties: a) the mRNA encoding each cancer antigen is interspersed by cleavage sensitive sites; b) the mRNA encoding each cancer antigen is linked directly to one another without a linker; c) the mRNA encoding each cancer antigen is linked to one another with a single nucleotide linker; d) each cancer antigen comprises a 20-40 amino acids and includes a centrally located SNP mutation; e) at least 40% of the cancer antigens have a highest affinity for class I MHC molecules from the subject; f) at least 40% of the cancer antigens have a highest affinity for class II MHC molecules from the subject; g) at least 40% of the cancer antigens have a predicted binding affinity of IC>500 nM for HLA-A, HLA-B and/or DRB1; h) the mRNA encodes 1 to 15 cancer antigens; i) 10-60% of the cancer antigens have a binding affinity for class I MHC and 10-60% of the cancer antigens have a binding affinity for class II MHC; and/or j) the mRNA encoding the cancer antigens is arranged such that the cancer antigens are ordered to minimize pseudo-epitopes.

In various embodiments, the combination comprising a compound of the invention and a cancer vaccine immunotherapeutic agent as disclosed herein can be used to illicit an immune response in a subject against a cancer antigen. The method involves administering to the subject a RNA vaccine comprising at least one RNA polynucleotide having an open reading frame encoding at least one antigenic polypeptide or an immunogenic fragment thereof, thereby inducing in the subject an immune response specific to the antigenic polypeptide or an immunogenic fragment thereof, in combination with administering a compound of the invention either in the same composition or a separate composition, administered at the same time, or sequentially dosed, wherein the anti-antigenic polypeptide antibody titer in the subject is increased following vaccination relative to anti-antigenic polypeptide antibody titer in a subject vaccinated with a prophylactically effective dose of a traditional vaccine against the cancer. An “anti-antigenic polypeptide antibody” is a serum antibody the binds specifically to the antigenic polypeptide.

A prophylactically effective dose is a therapeutically effective dose that prevents advancement of cancer at a clinically acceptable level. In some embodiments the therapeutically effective dose is a dose listed in a package insert for the vaccine. A traditional vaccine, as used herein, refers to a vaccine other than the mRNA vaccines of the invention. For instance, a traditional vaccine includes but is not limited to live microorganism vaccines, killed microorganism vaccines, subunit vaccines, protein antigen vaccines, DNA vaccines, and the like. In exemplary embodiments, a traditional vaccine is a vaccine that has achieved regulatory approval and/or is registered by a national drug regulatory body, for example the Food and Drug Administration (FDA) in the United States or the European Medicines Agency (EMA.)

In some embodiments the anti-antigenic polypeptide antibody titer in the subject is increased 1 log to 10 log following vaccination relative to anti-antigenic polypeptide antibody titer in a subject vaccinated with a prophylactically effective dose of a traditional vaccine against the cancer. In some embodiments the anti-antigenic polypeptide antibody titer in the subject is increased 1 log following vaccination relative to anti-antigenic polypeptide antibody titer in a subject vaccinated with a prophylactically effective dose of a traditional vaccine against the cancer. In some embodiments the anti-antigenic polypeptide antibody titer in the subject is increased 2 log following vaccination relative to anti-antigenic polypeptide antibody titer in a subject vaccinated with a prophylactically effective dose of a traditional vaccine against the cancer.

Aspects of the invention provide nucleic acid vaccines comprising one or more RNA polynucleotides having an open reading frame encoding a first antigenic polypeptide, wherein the RNA polynucleotide is present in the formulation for in vivo administration to a host, which confers an antibody titer superior to the criterion for sero-protection for the first antigen for an acceptable percentage of human subjects. In some embodiments, the antibody titer produced by the mRNA vaccines of the invention is a neutralizing antibody titer. In some embodiments the neutralizing antibody titer is greater than a protein vaccine. In other embodiments the neutralizing antibody titer produced by the mRNA vaccines of the invention is greater than an adjuvanted protein vaccine. In yet other embodiments the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

In preferred aspects, RNA vaccine immunotherapeutic agents of the present disclosure (e.g., mRNA vaccines) produce prophylactically—and/or therapeutically—efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, and the like.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.) In exemplary aspects of the invention, antigen-specific antibodies are measured in units of g/ml or are measured in units of IU/L (International Units per liter) or mIU/ml (milli International Units per ml). In exemplary embodiments of the invention, an efficacious vaccine produces >0.5 μg/mL, >0.1 μg/mL, >0.2 μg/mL, >0.35 μg/mL, >0.5 μg/mL, >1 μg/mL, >2 μg/mL, >5 μg/mL or >10 μg/mL. In exemplary embodiments of the invention, an efficacious vaccine produces >10 mIU/mL, >20 mIU/mL, >50 mIU/mL, >100 mIU/mL, >200 mIU/mL, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay. Also provided are nucleic acid vaccines comprising one or more RNA polynucleotides having an open reading frame encoding a first antigenic polypeptide or a concatemeric polypeptide, wherein the RNA polynucleotide is present in a formulation for in vivo administration to a host for eliciting a longer lasting high antibody titer than an antibody titer elicited by an mRNA vaccine having a stabilizing element or formulated with an adjuvant and encoding the first antigenic polypeptide. In some embodiments, the RNA polynucleotide is formulated to produce a neutralizing antibodies within one week of a single administration. In some embodiments, the adjuvant is selected from a cationic peptide and an immunostimulatory nucleic acid. In some embodiments, the cationic peptide is protamine.

Immunotherapeutic agents comprising a nucleic acid vaccine comprising one or more RNA polynucleotides having an open reading frame comprising at least one chemical modification or optionally no nucleotide modification, the open reading frame encoding a first antigenic polypeptide or a concatemeric polypeptide, wherein the RNA polynucleotide is present in the formulation for in vivo administration to a host such that the level of antigen expression in the host significantly exceeds a level of antigen expression produced by an mRNA vaccine having a stabilizing element or formulated with an adjuvant and encoding the first antigenic polypeptide.

Other aspects provide nucleic acid vaccines comprising one or more RNA polynucleotides having an open reading frame comprising at least one chemical modification or optionally no nucleotide modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

Aspects of the invention also provide a unit of use vaccine, comprising between 10 μg and 400 μg of one or more RNA polynucleotides having an open reading frame comprising at least one chemical modification or optionally no nucleotide modification, the open reading frame encoding a first antigenic polypeptide or a concatemeric polypeptide, and a pharmaceutically acceptable carrier or excipient, formulated for delivery to a human subject. In some embodiments, the vaccine further comprises a cationic lipid nanoparticle.

Aspects of the invention provide methods of creating, maintaining or restoring antigenic memory to a tumor in an individual or population of individuals comprising administering to said individual or population an antigenic memory booster nucleic acid vaccine comprising (a) at least one RNA polynucleotide, said polynucleotide comprising at least one chemical modification or optionally no nucleotide modification and two or more codon-optimized open reading frames, said open reading frames encoding a set of reference antigenic polypeptides, and (b) optionally a pharmaceutically acceptable carrier or excipient. In some embodiments, the vaccine is administered to the individual via a route selected from the group consisting of intramuscular administration, intradermal administration and subcutaneous administration. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

Aspects of the invention provide methods of vaccinating a subject comprising administering to the subject a single dosage of between 25 μg/kg and 400 μg/kg of a nucleic acid vaccine comprising one or more RNA polynucleotides having an open reading frame encoding a first antigenic polypeptide or a concatemeric polypeptide in an effective amount to vaccinate the subject.

Other aspects provide nucleic acid vaccines comprising one or more RNA polynucleotides having an open reading frame comprising at least one chemical modification, the open reading frame encoding a first antigenic polypeptide or a concatemeric polypeptide, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

In some embodiments, a compound of the invention can be used in combination with a bispecific antibody immunotherapeutic agent. The bispecific antibody can include a protein construct having a first antigen binding moiety and a second antigen binding site that binds to a cytotoxic immune cell. The first antigen binding site can bind to a tumor antigen that is specifically being treated with the combination of the present invention. For example, the first antigen binding moiety may bind to a non-limiting example of tumor antigens selected from: EGFR, HGFR, Her2, Ep-CAM, CD20, CD30, CD33, CD47, CD52, CD 133, CEA, gpA33, Mucins, TAG-72, CIX, PSMA, folate-binding protein, GD2, GD3, GM2, VEGF. VEGFR, Integrin αVβ3, Integrin α5β1, MUC1, ERBB2, ERBB3, MET, IGF1R, EPHA3, TRAILR1, TRAILR2, RANKL, FAP and Tenascin among others. In some embodiments, the first antigen binding moiety has specificity to a protein or a peptide that is overexpressed on a tumor cell as compared to a corresponding non-tumor cell. In some embodiments, the first antigen binding moiety has specificity to a protein that is overexpressed on a tumor cell as compared to a corresponding non-tumor cell. A “corresponding non-tumor cell” as used here, refers to a non-tumor cell that is of the same cell type as the origin of the tumor cell. It is noted that such proteins are not necessarily different from tumor antigens. Non-limiting examples include carcinoembryonic antigen (CEA), which is overexpressed in most colon, rectum, breast, lung, pancreas and gastrointestinal tract carcinomas; heregulin receptors (HER-2, neu or c-erbB-2), which is frequently overexpressed in breast, ovarian, colon, lung, prostate and cervical cancers; epidermal growth factor receptor (EGFR), which is highly expressed in a range of solid tumors including those of the breast, head and neck, non-small cell lung and prostate; asialoglycoprotein receptor; transferrin receptor; serpin enzyme complex receptor, which is expressed on hepatocytes; fibroblast growth factor receptor (FGFR), which is overexpressed on pancreatic ductal adenocarcinoma cells; vascular endothelial growth factor receptor (VEGFR), for anti-angiogenesis gene therapy; folate receptor, which is selectively overexpressed in 90% of nonmucinous ovarian carcinomas; cell surface glycocalyx; carbohydrate receptors; and polymeric immunoglobulin receptor.

The second antigen-binding moiety is any molecule that specifically binds to an antigen or protein or polypeptide expressed on the surface of a cytotoxic immune cell (a CIK cell). Exemplary non-limiting antigens expressed on the surface of the cytotoxic immune cells suitable for use with the present disclosure may include CD2, CD3, CD4, CD5, CD8, CD11a, CD11 b, CD 14, CD16a, CD27, CD28, CD45, CD45RA, CD56, CD62L, the Fc receptor, LFA, LFA-1, TCRαβ, CCR7, macrophage inflammatory protein 1a, perforin, PD-1, PD-L1, PD-L2, or CTLA-4, LAG-3, OX40, 41BB, LIGHT, CD40, GITR, TGF-beta, TIM-3, SIRP-alpha, TIGIT, VSIG8, BTLA, SIGLEC7, SIGLEC9, ICOS, B7H3, B7H4, FAS, BTNL2, CD27 and Fas ligand. In some embodiments, the second antigen binding moiety binds to CD3 of the cytotoxic immune cell, e.g., CIK cell. In some embodiments, the second antigen binding moiety binds to CD56 of the cytotoxic immune cell. In some embodiments, the second antigen binding moiety binds to the Fc receptor of the cytotoxic immune cell. In some embodiments, the Fc region of the bispecific antibody binds to the Fc receptor of the cytotoxic immune cell. In some embodiments, a second antigen-binding moiety is any molecule that specifically binds to an antigen expressed on the surface of a cytotoxic immune cell (e.g., a CIK cell). The second antigen binding moiety is specific for an antigen on a cytotoxic immune cell. Exemplary cytotoxic immune cells include, but are not limited to CIK cells, T-cells, CD8+ T cells, activated T-cells, monocytes, natural killer (NK) cells, NK T cells, lymphokine-activated killer (LAK) cells, macrophages, and dendritic cells. The second antigen binding moiety specifically binds to an antigen expressed on the surface of a cytotoxic immune cell. Exemplary non-limiting antigens expressed on the surface of the cytotoxic immune cells suitable for modulation with the present disclosure may include CD2, CD3, CD4, CD5, CD8, CD11a, CD11 b, CD14, CD16a, CD27, CD28, CD45, CD45RA, CD56, CD62L, the Fc receptor, LFA, LFA-1, TCRαβ, CCR7, macrophage inflammatory protein 1a, perforin, PD-1, PD-L1, PD-L2, or CTLA-4, LAG-3, OX40, 41BB, LIGHT, CD40, GITR, TGF-beta, TIM-3, SIRP-alpha, TIGIT, VSIG8, BTLA, SIGLEC7, SIGLEC9, ICOS, B7H3, B7H4, FAS, BTNL2, CD27 and Fas ligand. In other embodiments, the bispecific antibody modulator is an activator of a costimulatory molecule (e.g., an OX40 agonist). In one embodiment, the OX40 agonist is a bispecific antibody molecule to OX40 and another tumor antigen or a costimulatory antigen. The OX40 agonist can be administered alone, or in combination with other immunomodulators, e.g., in combination with an inhibitor (for example an antibody construct) of PD-1, PD-L1, CTLA-4, CEACAM (e.g., CEACAM-1, -3 and/or -5), TIM-3 or LAG-3. In some embodiments, the anti-OX40 antibody molecule is a bispecific antibody that binds to GITR and PD-1, PD-L1, CTLA-4, CEACAM (e.g., CEACAM-1, -3 and/or -5), TIM-3 or LAG-3. In one exemplary embodiment, an OX40 antibody molecule is administered in combination with an anti-PD-1 antibody molecule (e.g., an anti-PD-1 molecule as described herein). The OX40 antibody molecule and the anti-PD-1 antibody molecule may be in the form of separate antibody composition, or as a bispecific antibody molecule. In other embodiments, the OX40 agonist can be administered in combination with other costimulatory molecule, e.g., an agonist of GITR, CD2, CD27, CD28, CDS, ICAM-1, LFA-1 (CD11a/CD18), ICOS (CD278), 4-1BB (CD137), CD30, CD40, BAFFR, HVEM, CD7, LIGHT, NKG2C, SLAMF7, NKp80, CD 160, B7-H3, or CD83 ligand. In some embodiments, the second antigen binding moiety binds to the Fc receptor on the cytotoxic immune cell, e.g., CIK cell.

In some embodiments, the bispecific antibody immunotherapeutic agent has specificities for a tumor antigen and a CIK cell, which brings the tumor antigen expressing tumor cell in close proximity of the CIK cell, leading to the elimination of the tumor cell through anti-tumor cytotoxicity of CIK cell. In some embodiments, the bispecific antibody has specificity for a tumor antigen but does not have specificity for a CIK cell, however, the Fc region of the bispecific antibody can bind to the Fc receptor of the CIK cell, which in turn brings the tumor cell in close proximity of the CIK cell, leading to the elimination of the tumor cell through anti-tumor cytotoxicity of CIK cell. In some embodiments, the bispecific antibody has specificity for a CIK cell but does not have specificity for tumor cell, however, the Fc region of the bispecific antibody can bind to the Fc receptor of the tumor cell, which in turn brings the tumor cell in close proximity of the CIK cell, leading to the elimination of the tumor cell through anti-tumor cytotoxicity of CIK cell.

In some embodiments, a compound of the invention can be used in combination with an immune cell-engaging multivalent antibody/fusion protein/construct immunotherapeutic agent. In various embodiments, an exemplary immunotherapeutic agent can include immune cell-engaging multivalent antibody/fusion protein/construct which may comprise a recombinant structure, for example, all engineered antibodies that do not imitate the original IgG structure. Here, different strategies to multimerize antibody fragments are utilized. For example, shortening the peptide linker between the V domains forces the scFv to self-associate into a dimer (diabody; 55 kDa). Bispecific diabodies are formed by the noncovalent association of two VHA-VLB and VHB-VLA fragments expressed in the same cell. This leads to the formation of heterodimers with two different binding sites. Single-chain diabodies (sc-diabodies) are bispecific molecules where the VHA-VLB and VHB-VLA fragments are linked together by an additional third linker. Tandem-diabodies (Tandabs) are tetravalent bispecific antibodies generated by two scDiabodies.

Also included are the di-diabodies known in the art. This 130-kDa molecule is formed by the fusion of a diabody to the N-terminus of the CH3 domain of an IgG, resulting in an IgG-like structure. Further diabody derivatives are the triabody and the tetra-body, which fold into trimeric and tetrameric fragments by shortening the linker to <5 or 0-2 residues. Also exemplified are (scFv)₂ constructs known as ‘bispecific T cell engager’ (BITE). BITEs are bispecific single-chain antibodies consisting of two scFv antibody fragments, joined via a flexible linker, that are directed against a surface antigen on target cells and CD3 on T cells. Also exemplified are bivalent (Fab)2 and trivalent (Fab)3 antibody formats. Also exemplified are minibodies and trimerbodies generated from scFvs. Exemplary constructs useful to target tumor antigens as can include one or more of: Diabody, Single-chain (sc)-diabody (scFv)2, Miniantibody, Minibody, Bamase-barstar, scFv-Fc, sc(Fab)2, Trimeric antibody constructs, Triabody antibody constructs, Trimerbody antibody constructs, Tribody antibody constructs, Collabody antibody constructs, (scFv-TNFa)3, F(ab)3/DNL. Exemplary cytotoxic immune cells include, but are not limited to CIK cells, T-cells, CD8+ T cells, activated T-cells, monocytes, natural killer (NK) cells, NK T cells, lymphokine-activated killer (LAK) cells, macrophages, and dendritic cells.

In some embodiments, a compound of the invention can by used in combination with a radioconjugate immunotherapeutic agent.

In various embodiments, a radioconjugate is a small molecule or large molecule (herein referred to as a “cell targeting agent”), for example and polypeptide, an antibody or an antibody fragment thereof, that is coupled to or otherwise affixed to a radionuclide, or a plurality of radionuclides, such that the binding of the radioconjugate to its target (a protein or molecule on or in a cancer cell), will lead to the death or morbidity of said cancer cell. In various embodiments, the radioconjugate can be a cell targeting agent labelled with a radionuclide, or the cell targeting agent may be coupled or otherwise affixed to a particle, or microparticle, or nanoparticle containing a plurality of radionuclides, wherein the radionuclides are the same or different. Methods for synthesizing radioconjugates are known in the art, and may include the class of immunoglobulin or antigen binding parts thereof, that are conjugated to a toxic radionuclide.

In some embodiments, the molecule that binds to the cancer cell can be known as a “cell targeting agent”. As used herein, an exemplary cell targeting agent can allow the drug-containing nanoparticles or radionuclide to target the specific types of cells of interest. Examples of cell targeting agents include, but are not limited to, small molecules (e.g., folate, adenosine, purine) and large molecule (e.g., peptide or antibody) that bind to or target a tumor associated antigen. Examples of tumor associated antigens include, but are not limited to, adenosine receptors, alpha v beta 3, aminopeptidase P, alpha fetoprotein, cancer antigen 125, carcinoembryonic antigen, cCaveolin-1, chemokine receptors, clusterin, oncofetal antigens, CD20, epithelial tumor antigen, melanoma associated antigen, Ras, p53, Her2/Neu, ErbB2, ErbB3, ErbB4, folate receptor, prostate-specific membrane antigen, prostate specific antigen, purine receptors, radiation-induced cell surface receptor, serpin B3, serpin B4, squamous cell carcinoma antigens, thrombospondin, tumor antigen 4, tumor-associated glycoprotein 72, tyrosinase, and tyrosine kinases. In some embodiments, the cell targeting agent is folate or a folate derivative that binds specifically to folate receptors (FRs). In some embodiments, the cell targeting agent is an antibody, a bispecific antibody, a trispecific antibody or an antigen binding construct thereof, that specifically binds to a cancer antigen selected from: EGFR, HGFR, Her2, Ep-CAM, CD20, CD30, CD33, CD47, CD52, CD 133, CEA, gpA33, Mucins, TAG-72, CIX, PSMA, folate-binding protein, GD2, GD3, GM2, VEGF. VEGFR, Integrin αVβ3, Integrin α5β1, MUC1, ERBB2, ERBB3, MET, IGF1R, EPHA3, TRAILR1, TRAILR2, RANKL, FAP and Tenascin among others.

The use of folate as a targeting agent in the radioconjugate also allow both tumor cells and regulatory T (Treg) cells to be targeted for destruction. It is well accepted that high numbers of Treg cells suppress tumor immunity. Specifically, Treg cells suppress (foreign and self) reactive T cells without killing them through contact-dependent or cytokine (e.g., IL-10, TGF-.beta., and the like.) secretion. FR4 is selectively upregulated on Treg cells. It has been shown that antibody blockade of FR4 depleted Treg cells and provoked tumor immunity in tumor-bearing mice. Thus, folate-coated PBM nanoparticles carrying a cytotoxic agent would take FR-expressing cells for their destruction, which would both directly (i.e., BrCa cell) and indirectly (i.e., breast tumor associated and peripheral Treg cells) inhibit tumor progression.

In another further embodiment, the targeting agent is an antibody or peptide, or immune cell-engaging multivalent antibody/fusion protein/constructs capable of binding tumor associated antigens consisting of but not limited to: adenosine receptors, alpha v beta 3, aminopeptidase P, alpha fetoprotein, cancer antigen 125, carcinoembryonic antigen, caveolin-1, chemokine receptors, clusterin, oncofetal antigens, CD20, Human Growth Factor Receptor (HGFR), epithelial tumor antigen, melanoma associated antigen, MUC1, Ras, p53, Her2/Neu, ErbB2, ErbB3, ErbB4, folate receptor, prostate-specific membrane antigen, prostate specific antigen, purine receptors, radiation-induced cell surface receptor, serpin B3, serpin B4, squamous cell carcinoma antigens, thrombospondin, tumor antigen 4, tumor-associated glycoprotein 72, tyrosinase, tyrosine kinases, and the like.

In some embodiments, a compound as described herein can be used in combination with a vaccination protocol for the treatment of cancer. In some embodiments, a compound as described herein can be used in combination with an immunotherapeutic agent such as a vaccine. In various embodiments, exemplary vaccines include those used to stimulate the immune response to cancer antigens.

The amount of both the compound disclosed herein or salt thereof and the additional one or more additional therapeutic agent (in those compositions which comprise an additional therapeutic agent as described above) that may be combined with carrier materials to produce a single dosage form will vary depending upon the host treated and the particular mode of administration. In certain embodiments, compositions of this invention are formulated such that a dosage of between 0.01-100 mg/kg body weight/day of an inventive can be administered.

The additional therapeutic agent and the compound disclosed herein may act synergistically. Therefore, the amount of additional therapeutic agent in such compositions may be less than that required in a monotherapy utilizing only that therapeutic agent, or there may be fewer side effects for the patient given that a lower dose is used. In certain embodiments, in such compositions a dosage of between 0.01-10,000 μg/kg body weight/day of the additional therapeutic agent can be administered.

Labeled Compounds and Assay Methods

Another aspect of the present invention relates to labeled compounds of the invention (radio-labeled, fluorescent-labeled, etc.) that would be useful not only in imaging techniques but also in assays, both in vitro and in vivo, for localizing and quantitating protein kinases in tissue samples, including human, and for identifying protein kinase ligands by inhibition binding of a labeled compound. Accordingly, the present invention includes protein kinase assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I. The radionuclide that is incorporated in the instant radio-labeled compounds will depend on the specific application of that radio-labeled compound. For example, for in vitro metalloprotease labeling and competition assays, compounds that incorporate ³H, ¹⁴C, ⁸²Br, ¹²⁵I, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br, or ⁷⁷Br will generally be most useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S, and ⁸²Br.

The present invention can further include synthetic methods for incorporating radio-isotopes into compounds of the invention. Synthetic methods for incorporating radio-isotopes into organic compounds are well known in the art, and a person of ordinary skill in the art will readily recognize the methods applicable for the compounds of invention.

A labeled compound of the invention can be used in a screening assay to identify/evaluate compounds. For example, a newly synthesized or identified compound (i.e., test compound) which is labeled can be evaluated for its ability to bind a protein kinase by monitoring its concentration variation when contacting with the protein kinases, through tracking of the labeling. For example, a test compound (labeled) can be evaluated for its ability to reduce binding of another compound which is known to bind to a protein kinase (i.e., standard compound). Accordingly, the ability of a test compound to compete with the standard compound for binding to the protein kinase directly correlates to its binding affinity. Conversely, in some other screening assays, the standard compound is labeled, and test compounds are unlabeled. Accordingly, the concentration of the labeled standard compound is monitored in order to evaluate the competition between the standard compound and the test compound, and the relative binding affinity of the test compound is thus ascertained.

Synthesis

Compounds of this invention can be made by the synthetic procedures described below. The starting materials and reagents used in preparing these compounds are either available from commercial suppliers such as Sigma Aldrich Chemical Co. (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplemental (Elsevier Science Publishers, 1989); Organic Reactions, Volumes 1-40 (John Wiley and Sons, 1991); March's Advanced Organic Chemistry, (John Wiley and Sons, 4^{th} Edition); and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography, and the like. Such materials may be characterized using conventional means, including physical constants and spectral data.

Unless specified to the contrary, the reactions described herein take place at atmospheric pressure and over a temperature range from about −78° C. to about 150° C., more preferably from about 0° C. to about 125° C., and most preferably at about room (or ambient) temperature, for example, about 20° C. Unless otherwise stated (as in the case of a hydrogenation), all reactions are performed under an atmosphere of nitrogen.

The compounds disclosed and claimed herein may have asymmetric carbon atoms or quaternized nitrogen atoms in their structure and may be prepared through the syntheses described herein as single stereoisomers, racemates, or mixtures of enantiomers and diastereomers. The compounds may also exist as geometric isomers. All such single stereoisomers, racemates, and geometric isomers, and mixtures thereof are intended to be within the scope of this invention.

Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereomeric salts or complexes which may be separated, for example, by crystallization; via formation of diastereomeric derivatives which may be separated, for example, by crystallization; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomers may be synthesized by asymmetric synthesis using optically active reagents, substrates, catalysts, or solvents, or by converting on enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The methods of the present invention may be carried out as semi-continuous or continuous processes, more preferably as continuous processes.

The present invention as described above unless indicated otherwise may be carried out in the presence of a solvent or a mixture of two or more solvents. In particular the solvent is an aqueous or an organic solvent such as ether-like solvent (for example, tetrahydrofuran, methyltetrahydrofuran, diisopropyl ether, t-butylmethyl ether, or dibutyl ether), aliphatic hydrocarbon solvent (for example, hexane, heptane, or pentane), saturated alicyclic hydrocarbon solvent (for example, cyclohexane or cyclopentane), or aromatic solvent (for example, toluene, o-, m-, or p-xylene, or t-butyl-benzene) or mixture thereof.

The starting materials and reagents, which do not have their synthetic route explicitly disclosed herein, are generally available from commercial sources or are readily prepared using methods well known to the person skilled in the art.

Processes

One aspect provides a process of making a compound of formula IA:

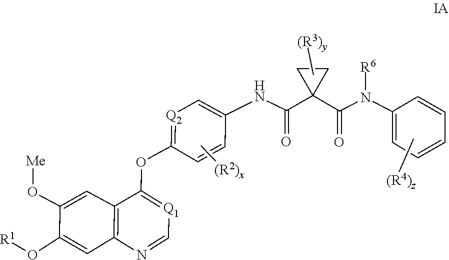

or a pharmaceutically acceptable salt thereof, comprising

reacting a compound of formula (A)

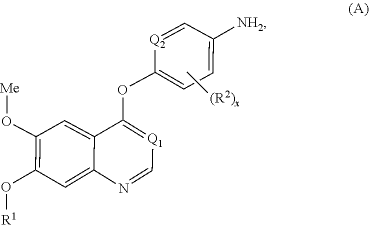

wherein R¹, R², Q₁, Q₂, and x are as defined in any embodiment of formula I or IA disclosed herein,

with a compound of formula (B)

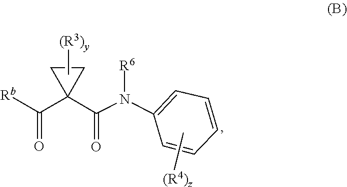

wherein R³, R⁴, y, z, are as defined in any embodiment of formula I or IA disclosed herein, R⁶ is methyl, and R^{b} is a leaving group,

to produce the compound of formula IA.

In some embodiments of this aspect, R^{b} is halo; in some instances, R^{b} is Cl.

Another aspect provides a process of making a compound of formula IIA:

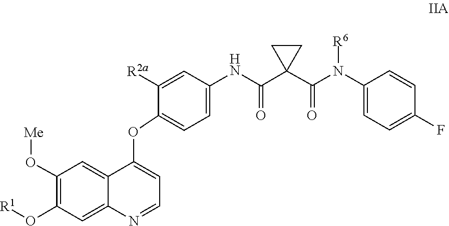

or a pharmaceutically acceptable salt thereof, comprising

reacting a compound of formula (C)

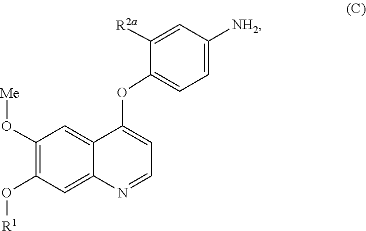

wherein R¹ and R^{2a} are as defined in any embodiment of formula II or IIA disclosed herein,

with a compound of formula (D)

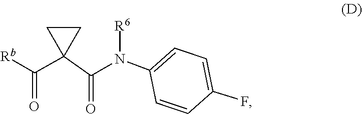

wherein R⁶ is methyl, and R^{b} is a leaving group,

to produce the compound of formula IIA.

In some embodiments of this aspect, R^{b} is halo; in some instances, R^{b} is Cl.

Another aspect provides a process comprising:

reacting a compound of formula (E)

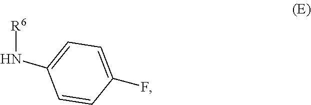

wherein R⁶ is methyl,

with a compound of formula (F)

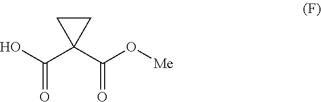

to produce a compound of formula (G)

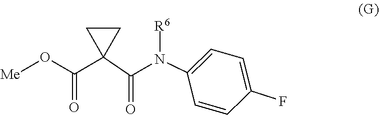

and optionally further reacting the compound of formula (G) with LiOH to form a compound of formula (H)

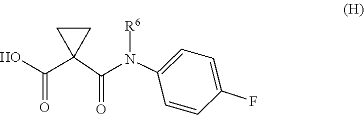

and optionally further reacting the compound of formula (H) with SOCl₂ to form a compound of formula (J)

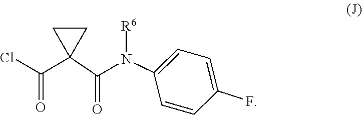

In some embodiments of this aspect, the reaction of formulas (E) and (F) is conducted in the presence of HATU and DIPEA.

In some embodiments of this aspect, R⁶ is methyl. In other embodiments, R⁶ is —CH₂OH. In other embodiments, R⁶ is —CH₂OCH₃.

Another aspect provides a process of making a compound of formula Ilia

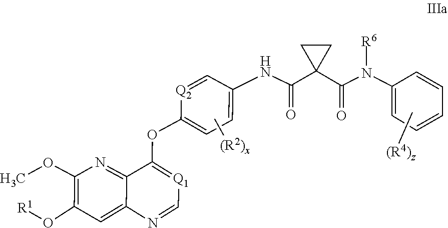

or a pharmaceutically acceptable salt thereof, comprising

reacting a compound of formula (K)

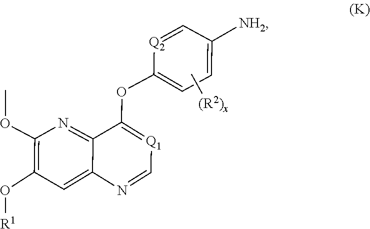

wherein R¹, R², Q₁, Q₂, and x are as defined in any embodiment of formula I′ or Ilia disclosed herein,

with a compound of formula (L)

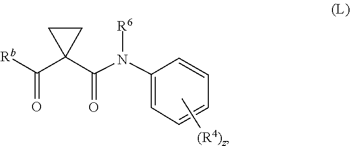

wherein R⁴ and z are as defined in any embodiment of formula I′ or Ilia disclosed herein, R⁶ is methyl, and R^{b} is a leaving group,

to produce the compound of formula Ilia.

Another aspect provides a method of producing a compound of formula IC or IC′:

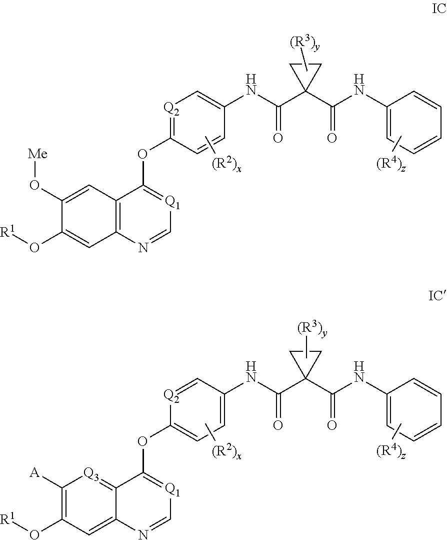

or a pharmaceutically acceptable salt thereof, comprising:

contacting a compound of formula I or I′ with a CYP450 enzyme, to produce a compound of formula IC or IC′, wherein the compounds of formula I and I′ have the structures:

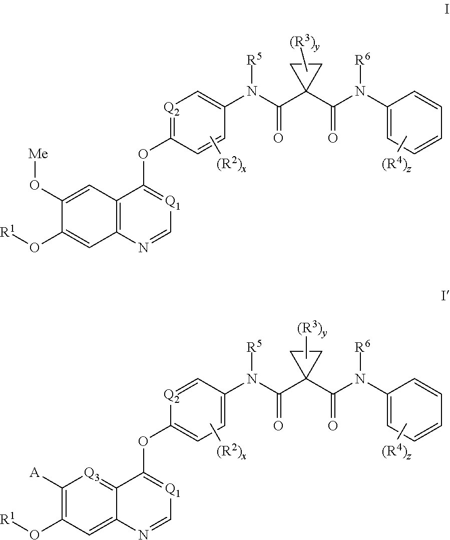

or a pharmaceutically acceptable salt thereof, wherein A, R¹, R², R³, R⁴, R⁵, R⁶, Q₁, Q₂, Q₃, x, y and z are as defined in any embodiment of formula I or formula I′ disclosed herein.

Another aspect provides a method of producing a compound of formula IE or IE′:

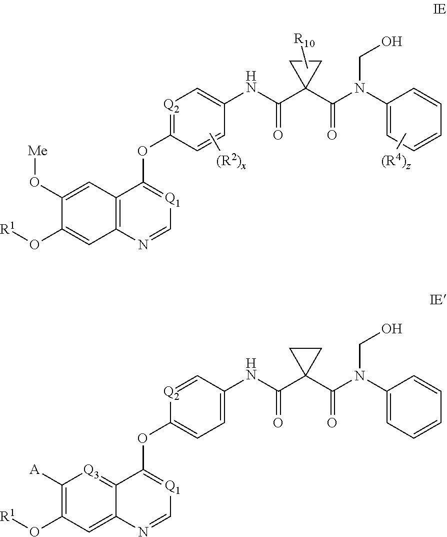

or a pharmaceutically acceptable salt thereof, comprising:

contacting a compound of formula ID or ID′ with a CYP450 enzyme, to produce a compound of formula IE or IE′, wherein the compounds of formula ID and ID′ have the structures:

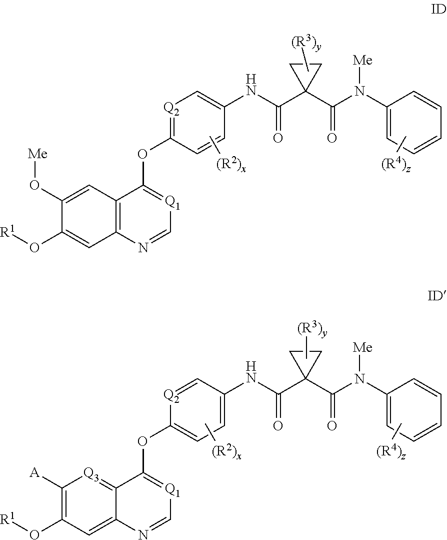

or a pharmaceutically acceptable salt thereof, wherein A, R¹, R², R³, R⁴, Q₁, Q₂, Q₃, x, y and z are as defined in any embodiment of formula I or formula I′ disclosed herein.

Another aspect provides a method of producing a compound of formula IG or IG′:

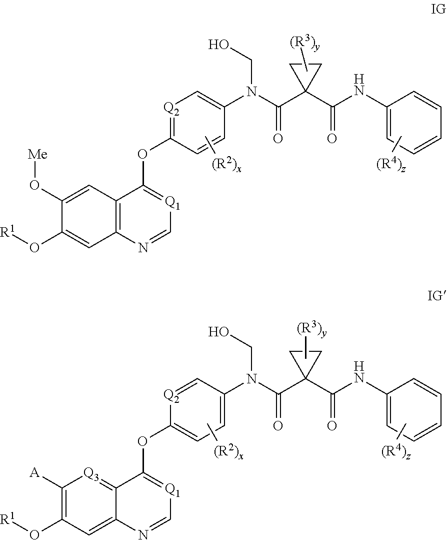

or a pharmaceutically acceptable salt thereof, comprising:

contacting a compound of formula IF or IF′ with a CYP450 enzyme, to produce a compound of formula IG or IG′, wherein the compounds of formula IF and IF′ have the structures:

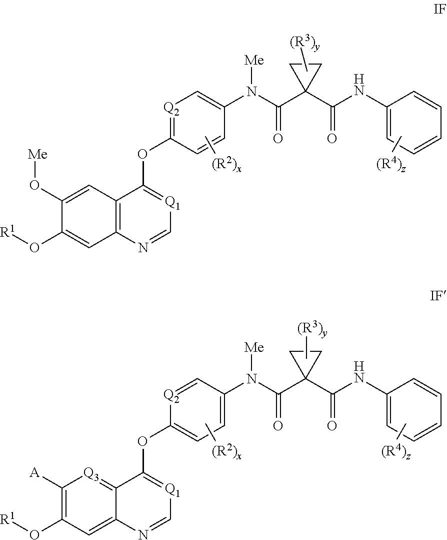

or a pharmaceutically acceptable salt thereof, wherein A, R¹, R², R³, R⁴, Q₁, Q₂, Q₃, x, y and z are as defined in any embodiment of formula I or formula I′ disclosed herein.

In some embodiments of this aspect, the process is conducted in the presence of an organic solvent.

### EXAMPLES

The following examples are provided for the purpose of further illustration and are not intended to limit the scope of the claimed invention.

### Example 1: 4-((6,7-Dimethoxyquinolin-4-yl)oxy)aniline (3)

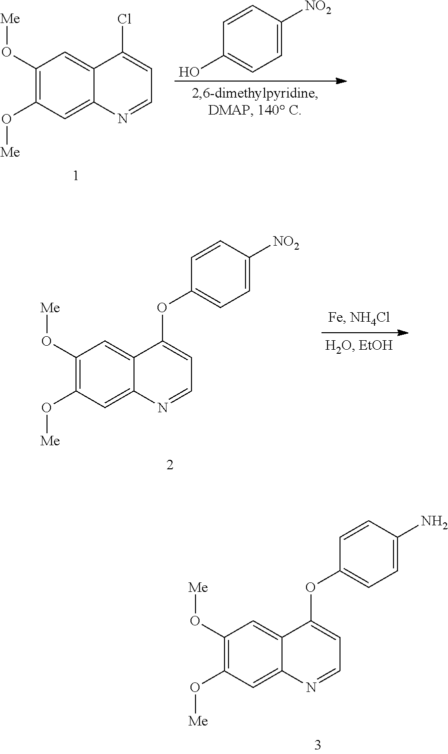

### 6,7-Dimethoxy-4-(4-nitrophenoxy)quinoline (2)

To a mixture of Compound 1 (10 g, 44.7 mmol, 1 eq) and 4-nitrophenol (8.70 g, 62.5 mmol, 1.4 eq) in 2,6-dimethylpyridine (50 mL) was added DMAP (1.10 g, 9.0 mmol, 2.01e-1 eq). The mixture was stirred at 140° C. for 36 h. The reaction was cooled to room temperature, MeOH (32 g) was added, followed by aq K₂CO₃ (4 g in water (62 g)). The resulting mixture was stirred at 0° C. for 2 h. The resulting precipitate was filtered and washed with water (200 mL) to give Compound 2 as a yellow solid (8.0 g, 54.8% yield). ¹H NMR (400 MHz, CDCl₃) δ 8.63 (d, 1H), 8.39-8.28 (m, 2H), 7.49 (s, 1H), 7.38 (s, 1H), 7.30-7.15 (m, 1H), 7.26 (s, 1H), 6.70 (d, 1H), 4.07 (s, 3H), 4.01 (s, 3H); MS (EI) for C₁₇H₁₄N₂O₅, found 326.8 (MH+).

4-((6,7-Dimethoxyquinolin-4-yl)oxy)aniline (3): To a mixture of Compound 2 (2.0 g, 6.1 mmol, 1 eq) in EtOH (40 mL) and water (8 mL) was added Fe (1.71 g, 30.6 mmol, 5.0 eq) and NH₄Cl (2.62 g, 49.0 mmol, 8.0 eq). The mixture was stirred at 85° C. for 3 h. The reaction was filtered, and the filtrate was dried over anhyd Na₂SO₄ and concentrated to give crude product. To this crude product was added EtOAc (150 mL) and DCM (150 mL). The resulting mixture was filtered, and the filtrate was concentrated to give Compound 3 as a yellow solid (1.1 g, 60.6% yield). ¹H NMR (400 MHz, DMSO-d₆) δ 8.42 (d, 1H), 7.50 (s, 1H), 7.36 (s, 1H), 6.99-6.84 (m, 2H), 6.74-6.56 (m, 2H), 6.36 (d, 1H), 5.16 (s, 2H), 3.93 (d, 6H); MS (EI) for C₁₇H₁₆N₂O₃, found 297.2 (MH+).

### Example 2: 1-((4-Fluorophenyl)(methyl)carbamoyl)cyclopropane-1-carbonyl Chloride (8)

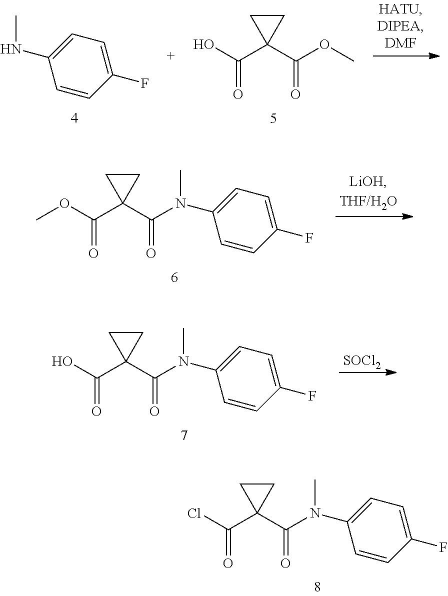

### Methyl 1-((4-fluorophenyl)(methyl)carbamoyl)cyclopropane-1-carboxylate (6)

HATU (73 g, 192.0 mmol, 1.2 eq) was added to a solution of Compound 4 (20 g, 159.8 mmol, 19.23 mL, 1 eq), Compound 5 (23.03 g, 159.82 mmol, 1 eq), and DIPEA (59 g, 456.5 mmol, 79.51 mL, 2.9 eq) in DMF (100 mL). The reaction mixture was stirred at 10-20° C. for 17 h. The mixture was diluted with water (500 mL) and extracted with EtOAc (2×500 mL). The combined organic extracts were washed with aq saturated NaCl (3×100 mL), dried over anhyd Na₂SO₄, and concentrated under vacuum to give crude Compound 6 as a brown oil (85 g), which was used subsequent reactions without further purification. MS (EI) for C₁₃H₁₄FNO₃, found 251.9 (MH+).

### 1-((4-Fluorophenyl)(methyl)carbamoyl)cyclopropane-1-carboxylic acid (7)

To a solution of Compound 6 (40 g, 79.6 mmol, 1 eq) in THF (200 mL) and water (40 mL) was added LiOH H₂O (6.68 g, 159.2 mmol, 2 eq). The mixture was stirred at 50° C. for 6 h. The mixture was concentrated under vacuum to remove the organic solvents. The resulting aqueous mixture was washed with EtOAc (300 ml) and then acidified to pH 4-5 with aq HCl (12M). The resulting precipitate was collected by filtration and dried under vacuum to give Compound 7 as a yellow solid (9.0 g, 37.56 mmol, 47.2% yield). ¹H NMR (400 MHz, DMSO-d₆) δ 12.53 (br s, 1H), 7.35 (br d, 2H), 7.23-7.19 (m, 2H), 3.13 (s, 3H), 1.20 (br s, 2H), 0.96 (br s, 2H); MS (EI) for C₁₂H₁₂FNO₃, found 237.8 (MH+).

### 1-((4-Fluorophenyl)(methyl)carbamoyl)cyclopropane-1-carbonyl chloride (8)

A mixture of Compound 7 (1.3 g, 5.48 mmol, 1 eq.) in SOCl₂ (20 mL) was stirred at 85° C. for 12 h. The reaction mixture was concentrated under reduced pressure and co-evaporated with anhydrous DCM (4×30 mL) to give Compound 8 as a brown oil (1.3 g, 92.8% yield) which was used in subsequent reactions without further purification. MS (EI) after test quench with methanol to give the corresponding methyl ester C₁₃H₁₄FNO₃, found 252.1 (MH+).

Alternatively, Compound 8 can be synthesized using the same methods used to synthesize the related compound, 1-((4-fluorophenyl)carbamoyl)cyclopropane-1-carbonyl chloride, as described previously in WO2012109510 A1 and WO2010051373 A1, replacing 4-flouroaniline with 4-fluoro-N-methylaniline.

### Example 3: 1-N-[4-(6,7-Dimethoxyquinolin-4-yl)oxyphenyl]-1-N′-(4-fluorophenyl)-1-N′-methylcyclopropane-1,1-dicarboxamide hydrochloride (9)

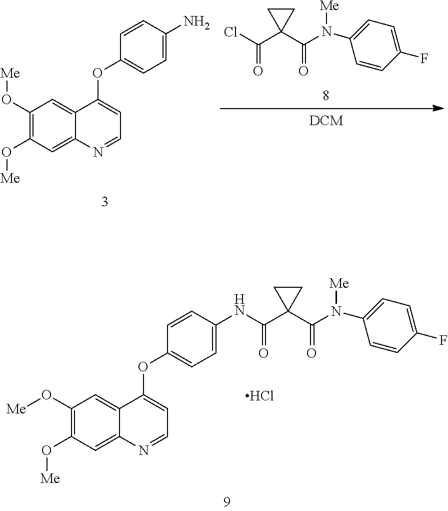 [SMILES: COc1cc2nccc(Oc3ccc(N)cc3)c2cc1OC ; CN(C(=O)C1(C(=O)Cl)CC1)c1ccc(F)cc1 ; COc1cc2nccc(Oc3ccc(NC(=O)C4(C(=O)N(C)c5ccc(F)cc5)CC4)cc3)c2cc1OC ; Cl]

### 1-N-[4-(6,7-Dimethoxyquinolin-4-yl)oxyphenyl]-1-N′-(4-fluorophenyl)-1-N′-methylcyclopropane-1,1-dicarboxamide hydrochloride (9)

To a mixture of Compound 3 (1.5 g, 5.1 mmol, 1 eq) in DCM (30 mL) was added Compound 8 (1.3 g, 5.1 mmol, 1.0 eq). The mixture was stirred at 6-11° C. for 12 h. The reaction mixture was filtered, and the resulting solid was washed with DCM (3×50 mL) and dried to give the hydrochloride salt of Compound 9 as a gray solid (1.76 g, 63.2% yield). ¹H NMR (400 MHz, DMSO-d₆) δ 9.83 (br s, 1H), 8.82 (d, 1H), 7.74 (s, 2H), 7.56 (br s, 2H), 7.33-7.27 (m, 4H), 7.10 (t, 2H), 6.79 (d, 1H), 4.04 (s, 6H), 3.25 (s, 3H), 1.45-1.39 (m, 2H), 1.25 (br s, 2H); MS (EI) for C₂₉H₂₆FN₃O₅, found 516.3 (MH+).

### Example 4: N-(4-((6,7-Dimethoxyquinolin-4-yl)oxy)-3-fluorophenyl)-N-(4-fluorophenyl)-N-methylcyclopropane-1,1-dicarboxamide (10)

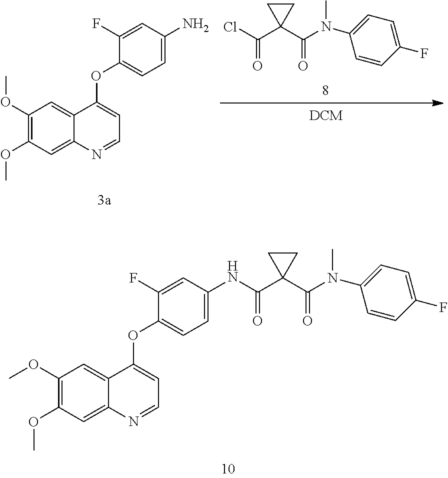 [SMILES: COc1cc2nccc(Oc3ccc(N)cc3F)c2cc1OC ; CN(C(=O)C1(C(=O)Cl)CC1)c1ccc(F)cc1 ; COc1cc2nccc(Oc3ccc(NC(=O)C4(C(=O)N(C)c5ccc(F)cc5)CC4)cc3F)c2cc1OC]

### N-(4-((6,7-Dimethoxyquinolin-4-yl)oxy)-3-fluorophenyl)-N-(4-fluorophenyl)-N-methylcyclopropane-1,1-dicarboxamide (10)

A solution of Compound 8 (1.08 g, 4.22 mmol, 1.33 eq.) and Compound 3a (1.0 g, 3.18 mmol, 1 eq.) in CH₂Cl₂ (10 mL) was stirred at 10-20° C. for 16 h. The mixture was diluted with aq. NaHCO₃ (30 mL) and extracted with DCM (3×30 mL). The combined organic layers were dried over anhyd Na₂SO₄ and concentrated under vacuum. The resulting residue was purified by column chromatography on silica gel (50%-100% EtOAc in petroleum), concentrated and lyophilized to give Compound 10 as a white solid (957.0 mg, 56.4% yield). ¹H NMR (400 MHz, DMSO-d₆) δ 9.93 (br s, 1H), 8.49 (d, 1H), 7.52 (s, 1H), 7.48 (br d, 1H), 7.41 (s, 1H), 7.38-7.32 (m, 1H), 7.28 (br d, 3H), 7.10 (br t, 2H), 6.41 (d, 1H), 3.95 (s, 6H), 3.24 (s, 3H), 1.47-1.40 (m, 2H), 1.30-1.18 (m, 2H); MS (EI) for C₂₉H₂₅F₂N₃O₅, found 534.0 (MH+). Compound 3a can be made from Compound 1 and 4-nitro-2-fluorophenol in the same manner that Compound 3 was made from Compound 1 and 4-nitrophenol in Example 1.

### Example 5

N-(3-Fluoro-4-((6-methoxy-7-(3-morpholinopropoxy)quinolin-4-yl)oxy)phenyl)-N-(4-fluorophenyl)-N-methylcyclopropane-1,1-dicarboxamide (11) was prepared according to Example 4 using 3-fluoro-4-[6-methoxy-7-(3-morpholin-4-yl-propoxy)-quinolin-4-yloxy]-phenylamine (US2013/0197230) in place of Compound 3. ¹H NMR (400 MHz, CDCl₃) δ 8.54 (s, 1H), 8.48 (d, 1H), 7.69-7.63 (m, 1H), 7.57 (s, 1H), 7.44 (s, 1H), 7.23-7.19 (m, 2H), 7.15-7.09 (m, 4H), 6.39 (d, 1H), 4.28 (t, 2H), 4.04 (s, 3H), 3.73 (t, 4H), 3.38 (s, 3H), 2.61-2.55 (m, 2H), 2.49 (s, 4H), 2.13 (quin, 2H), 1.38-1.32 (m, 2H), 1.13-1.07 (m, 2H); MS (EI) for C₃₅H₃₆F₂N₄O₆, found 669.1 [M+Na]⁺.

### Example 8: 4-((3-Fluoro-5-(l-((4-fluorophenyl)(methyl)carbamoyl)cyclopropane-1-carboxamido)pyridin-2-yl)oxy)-7-methoxyquinoline-6-carboxylic Acid (33)

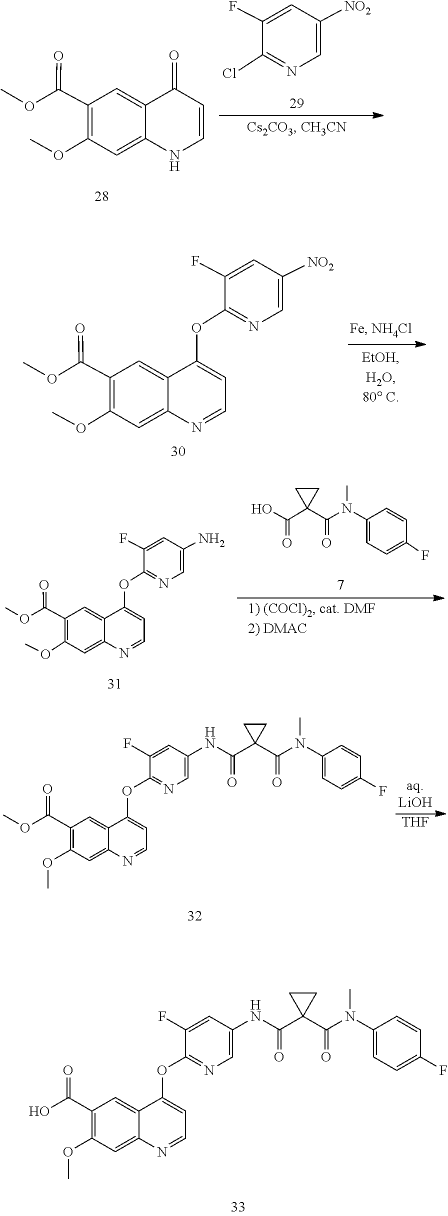

### Methyl 4-((3-fluoro-5-nitropyridin-2-yl)oxy)-7-methoxyquinoline-6-carboxylate (30)

To a solution of Compound 28 (5.0 g, 21.44 mmol, 1 eq) in CH₃CN (80 mL) was added Cs₂CO₃ (13.97 g, 42.88 mmol, 2 eq) in one portion at 16° C. The mixture was stirred at 16° C. for 30 min. Compound 29 (4.54 g, 25.73 mmol, 1.2 eq) was added. The mixture was stirred at 16° C. for 12 h. The resulting solid was filtered and washed with 150 mL of EtOAc. The filter cake was diluted with water (200 mL) and extracted with DCM (3×150 mL). The combined organic phases were washed with aq saturated NaCl (50 mL), filtered and concentrated under reduced pressure to give Compound 30 as a yellow solid (3.5 g, 43.7% yield). ¹H NMR (400 MHz, CDCl₃) δ 8.93 (d, 1H), 8.85 (d, 1H), 8.46 (s, 1H), 8.42 (dd, 1H), 7.59 (s, 1H), 7.22 (d, 1H), 4.06 (s, 3H), 3.95 (s, 3H); MS (EI) for C₁₇H₁₂FN₃O₆, found 373.9 (MH+).

### Methyl 4-((5-amino-3-fluoropyridin-2-yl)oxy)-7-methoxyquinoline-6-carboxylate (31)

To a mixture of Compound 30 (3.5 g, 9.38 mmol, 1 eq) in water (5 mL) and EtOH (40 mL) was added Fe (2.62 g, 46.88 mmol, 5 eq) and NH₄Cl (5.02 g, 93.76 mmol, 10 eq) and the mixture was stirred at 80° C. for 2 h. EtOH (250 mL) was added and the resulting suspension was filtered through a pad of Celite®. The filter cake was washed with EtOH (3×80 mL). The filtrate was concentrated to dryness, washed with water (50 mL) and dried in vacuo to give Compound 31 as a yellow solid (2.5 g, 77.67% yield) which was used in subsequent reactions without further purification. MS (EI) for C₁₇H₁₄FN₃O₄, found 343.9 (MH+).

### Methyl 4-((3-fluoro-5-(l-((4-fluorophenyl)(methyl)carbamoyl)cyclopropane-1-carboxamido)pyridin-2-yl)oxy)-7-methoxyquinoline-6-carboxylate (32)

Compound 7 (600 mg, 2.53 mmol, 1 eq) was suspended in anhydrous DCM (8 mL) at 10° C. and (COCl)₂ (321.02 mg, 2.53 mmol, 221.40 μL, 1 eq) was added with stirring under nitrogen, followed by DMF (18.49 mg, 252.92 μmol, 19.46 μL, 0.1 eq). The mixture was stirred at 10° C. for 1 h. The sample was quenched with benzyl amine (BnNH₂). The solvent was removed under reduced pressure and the resulting crude acid chloride was slowly added to a solution of Compound 31 (600 mg, 1.75 mmol, 1 eq) in DMAC (8 mL). The resulting reaction mixture was stirred at 10° C. for 1 h then poured into aq saturated NH₄Cl (50 mL) and extracted with DCM (3×30 mL). The combined organic phases were washed with aq saturated NaHCO₃ (20 mL), aq saturated NaCl (10 mL), dried over anhyd Na₂SO₄ and concentrated in vacuo to give Compound 32 as a yellow solid (730 mg, 74.2% yield). MS (EI) for C₂₉H₂₄F₂N₄O₆, found 563.5 (MH+).

### 4-((3-Fluoro-5-(l-((4-fluorophenyl)(methyl)carbamoyl)cyclopropane-1-carboxamido)pyridin-2-yl)oxy)-7-methoxyquinoline-6-carboxylic acid (33)

To a mixture of Compound 32 (730 mg, 1.30 mmol, 1 eq) in water (10 mL) and THF (2 mL) was added LiOH (2 M, 3.24 mL, 5 eq) slowly and the reaction mixture was stirred at 10° C. for 1 h. The reaction mixture was concentrated and the residue was diluted with water (20 mL) and acidified with aq HCl (1 M) until pH=3. The resulting solid was filtered and washed with H₂O (2.0 ml to give Compound 33 as a yellow solid (600 mg, 84.3% yield). MS (EI) for C₂₈H₂₂F₂N₄O₆, found 549.0 (MH+).

### Example 9: 1-N-[5-Fluoro-6-[7-methoxy-6-(methylcarbamoyl)quinolin-4-yl]oxypyridin-3-yl]-1-N′-(4-fluorophenyl)-1-N′-methylcyclopropane-1,1-dicarboxamide (34)

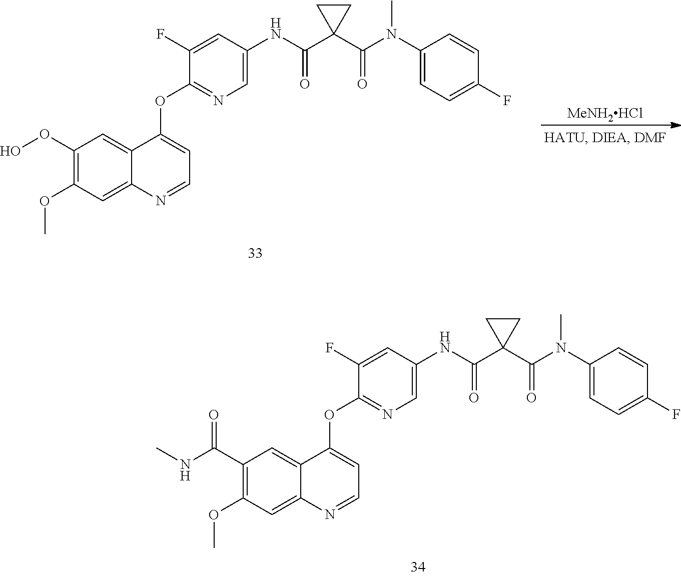 [SMILES: "MeNH2·HCl" ; COc1cc2nccc(Oc3ncc(NC(=O)C4(C(=O)N(C)c5ccc(F)cc5)CC4)cc3F)c2cc1OO ; CNC(=O)c1cc2c(Oc3ncc(NC(=O)C4(C(=O)N(C)c5ccc(F)cc5)CC4)cc3F)ccnc2cc1OC]

### 1-N-[5-Fluoro-6-[7-methoxy-6-(methylcarbamoyl)quinolin-4-yl]oxypyridin-3-yl]-1-N′-(4-fluorophenyl)-1-N′-methylcyclopropane-1,1-dicarboxamide (34)

To a solution of Compound 33 (200 mg, 364.64 μmol, 1 eq) in DMF (3 mL) was added HATU (152.51 mg, 401.10 μmol, 1.1 eq) and DIEA (141.38 mg, 1.09 mmol, 190.54 μL, 3 eq) and stirred at 10° C. for 30 min. Methanamine hydrochloride (73.86 mg, 1.09 mmol, 3 eq) was added and the reaction mixture was stirred at 10° C. for 12 h. The reaction mixture was poured into water (30 mL) and extracted with DCM (3×20 mL). The combined organic phases were washed with aq saturated NaCl (10 mL), concentrated in vacuo and the resulting residue purified by prep-HPLC(Column: HT C18 Highload 150 mm*25 mm*5 um, gradient: 24-54% of acetonitrile in water (0.04% NH₃.H₂O+10 mM NH₄HCO₃), flow rate: 30 mL/min to give Compound 34 as a yellow solid (81.6 mg, 39.8% yield). ¹H NMR (400 MHz, DMSO-d₆) δ 8.78 (d, 1H), 8.45 (s, 1H), 8.38 (br d, 1H), 8.08 (br s, 1H), 7.88 (br s, 1H), 7.56 (s, 1H), 7.33-7.24 (m, 2H), 7.17-7.09 (m, 2H), 6.91 (d, 1H), 4.03 (s, 3H), 3.24 (s, 3H), 2.83 (d, 3H), 1.48-1.41 (m, 2H), 1.23 (br s, 2H); MS (EI) for C₂₉H₂₅F₂N₅O₅, found 562.0 (MH+).

### Example 10

1-N-[6-(6-Carbamoyl-7-methoxyquinolin-4-yl)oxy-5-fluoropyridin-3-yl]-1-N′-(4-fluorophenyl)-1-N′-methylcyclopropane-1,1-dicarboxamide (35) was prepared according to Example 9 using NH₄Cl in place of the methanamine hydrochloride. ¹H NMR (400 MHz, DMSO-d₆) δ 10.21 (br s, 1H), 8.78 (d, 1H), 8.53 (s, 1H), 8.09 (br s, 1H), 7.93-7.70 (m, 3H), 7.56 (s, 1H), 7.35-7.22 (m, 2H), 7.18-7.08 (m, 2H), 6.91 (d, 1H), 4.04 (s, 3H), 3.24 (s, 3H), 1.50-1.39 (m, 2H), 1.23 (br s, 2H); MS (EI) for C₂₈H₂₃F₂N₅O₅, found 548.0 (MH+).

### Example 11: 1-N-[4-[(6,7-Dimethoxy-1,5-naphthyridin-4-yl)oxy]-3-fluorophenyl]-1-N′-(4-fluorophenyl)-1-N′-methylcyclopropane-1,1-dicarboxamide (44)

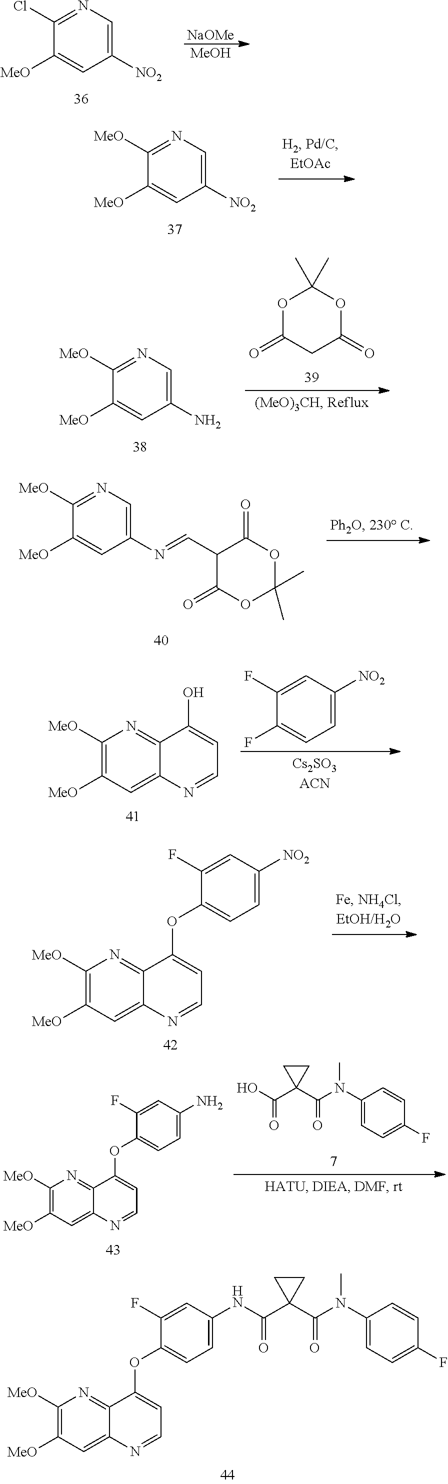

### 2,3-Dimethoxy-5-nitropyridine (37)

Freshly cut sodium (0.6 g, 26 mmol) was added portion wise to MeOH (50 mL) and the mixture was stirred at room temperature until the sodium dissolved. Compound 36 (3.0 g, 15.9 mmol) was added and the reaction mixture was stirred at room temperature for 1 h. Water (100 mL) was added and the mixture was filtered. The solids were washed with water and dried to give Compound 37 (2.78 g, 95% yield). MS for C₇H₈N₂O₄, found 185 (MH+).

### 2,3-Dimethoxy-5-nitropyridine (38)

To a solution of Compound 37 (2.78 g, 15.1 mmol) in EtOAc (40 mL) under argon was added 10% Pd/C (53% water, 880 mg). The reaction mixture was stirred under one atmosphere of H₂ at room temperature overnight and then filtered through Celite®. The filtrate was concentrated under vacuum to provide crude Compound 38 as a brown solid (2.31 g, 100% yield). MS for C₇H₁₀N₂O₂, found 155 (MH+).

### 5-(((5,6-Dimethoxypyridin-3-yl)imino)methyl)-2,2-dimethyl-1,3-dioxane-4,6-dione (40)

A solution of triethyl orthoformate (12 mL) and Compound 39 (1.44 g, 10.0 mmol) was stirred at 106° C. for 2.5 h, followed by the addition of Compound 38 (1.54 g, 10.0 mmol) while maintaining the same temperature. A precipitate appeared within several minutes. The heterogeneous mixture was heated at 105° C. for an additional 10 min, cooled to room temperature and filtered. The solids were washed with hexanes and dried to give crude Compound 40 (3.6 g). MS for C₁₄H₁₆N₂O₆, found 309 (MH+).

### 6,7-Dimethoxy-1,5-naphthyridin-4-ol (41)

A solution of Compound 40 (1.55 g, 5.03 mmol) in diphenyl ether (12 mL) was heated at 250° C. for 30 min, and then cooled to room temperature. Diethyl ether was added and the mixture was filtered to give crude Compound 40 as a brown solid (0.92 g, 89% yield). MS for C₁₀H₁₀N₂O₃, found 207 (MH+).

### 8-(2-Fluoro-4-nitrophenoxy)-2,3-dimethoxy-1,5-naphthyridine (42)

A mixture of Compound 41 (1.0 g, 4.8 mmol), 1,2-difluoro-4-nitrobenzene (0.93 g, 6.8 mmol), and Cs₂CO₃ (6.6 g, 20 mmol) in acetonitrile (20 mL) was stirred at room temperature overnight. EtOAc (80 mL) was added and the resulting mixture was filtered. The filtrate was evaporated in vacuo and the resulting residue was purified by silica gel chromatography to give Compound 42 (670 mg, 40% yield). MS for C₁₆H₁₂FN₃O₅, found 346 (MH+).

### 4-((6,7-Dimethoxy-1,5-naphthyridin-4-yl)oxy)-3-fluoroaniline (43)

A mixture of Compound 42 (620 mg, 1.8 mmol), NH₄Cl (500 mg, 9.3 mmol), and Fe (260 mg, 4.6 mmol) in MeOH/water (20/5 mL) was refluxed for 1 h and cooled to room temperature. The mixture was filtered through Celite® and the filtrate was concentrated to remove MeOH. To the residue was added aq saturated NaHCO₃ (6 mL) and the resulting mixture was extracted with EtOAc. The organic extract was dried over anhyd Na₂SO₄ and concentrated in vacuo to give crude Compound 43 as a brown solid (530 mg, 94% yield). MS for C₁₆H₁₄FN₃O₃, found 316 (MH+).

### 1-N-[4-[(6,7-Dimethoxy-1,5-naphthyridin-4-yl)oxy]-3-fluorophenyl]-1-N′-(4-fluorophenyl)-1-N′-methylcyclopropane-1,1-dicarboxamide (44)

To a mixture of Compound 43 (31 mg, 0.10 mmol) and Compound 7 (46 mg, 0.20 mmol) in DMF (1 mL) was added HATU (120 mg, 0.32 mmol) followed by DIEA (0.10 mL, 0.57 mmol). The reaction was stirred at room temperature overnight. Aq saturated NaHCO₃ (2 mL) and water (2 mL) were added and resulting suspension was filtered. The solid was purified by silica gel chromatography followed by prep-HPLC to give Compound 44 (12 mg, 23% yield). ¹H NMR (400 MHz, CDCl₃) δ 8.71 (s, 1H), 8.42 (d, 1H), 8.25 (s, 1H), 7.74-7.59 (m, 1H), 7.22 (dd, 2H), 7.04 (s, 2H), 7.02 (d, 2H), 6.82 (dd, 1H), 4.15 (s, 3H), 4.08 (s, 3H), 3.30 (s, 3H), 1.28 (q, 2H), 1.02 (q, 2H); MS for C₂₈H₂₄F₂N₄O₅, found 535 (MH+).

### Example 12: 1-N-[4-(6,7-Dimethoxyquinolin-4-yl)oxyphenyl]-1-N′-(4-fluorophenyl)-1-N′-(methoxymethyl)cyclopropane-1,1-dicarboxamide (49)

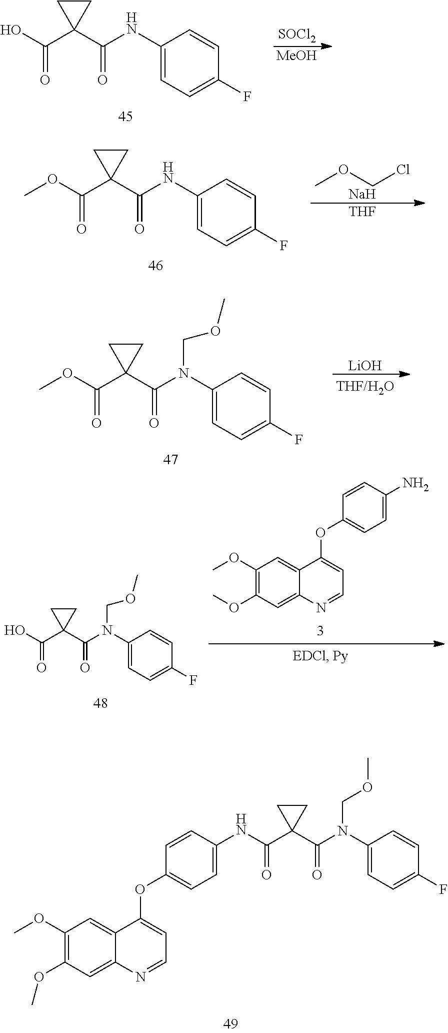

### Methyl 1-((4-fluorophenyl)carbamoyl)cyclopropane-1-carboxylate (46)

To a solution of Compound 45 (1.00 g, 4.48 mmol, 1 eq) in MeOH (10 mL) was added SOCl₂ (5.33 g, 44.80 mmol, 3.25 mL, 10 eq) at 0° C. The mixture was stirred at 65° C. for 2 h. The reaction mixture was concentrated under reduced pressure and the resulting residue was purified by silica gel column chromatography (PE/EtOAc=1/0 to 3/1) to give Compound 46 as an off-white solid (550 mg, 51.8% yield). MS for C₁₂H₁₂FNO₃, found 237.9 (MH+).

### Methyl 1-((4-fluorophenyl)(methoxymethyl)carbamoyl)cyclopropane-1-carboxylate (47)

To a solution of Compound 46 (400 mg, 1.69 mmol, 1 eq) in THF (5 mL) was added NaH (202.32 mg, 5.06 mmol, 60% purity, 3.0 eq) at 0° C. The reaction mixture was stirred at 10° C. for 0.5 h. Chloro(methoxy)methane (678.79 mg, 8.43 mmol, 640.37 μL, 5.0 eq) was added and the resulting mixture was stirred at 10° C. for 12 h under an atmosphere of nitrogen. Water (20 mL) was added and the resulting mixture was extracted with EtOAc (3×20 mL). The combined organic extracts were washed with water (10 mL), aq saturated NaCl (10 mL), dried over anhyd Na₂SO₄ and concentrated under reduced pressure to give crude Compound 47 as a yellow oil (474 mg) which was used in subsequent reactions without further purification. MS for C₁₄H₁₆FNO₄, found 303.9 [M+Na]⁺.

### 1-((4-Fluorophenyl)(methoxymethyl)carbamoyl)cyclopropane-1-carboxylic Acid (48)

To a solution of Compound 47 (474 mg, 1.69 mmol, 1 eq) in THF (3 mL) and water (3 mL) was added LiOH H₂O (282.86 mg, 6.74 mmol, 4.0 eq). The mixture was stirred at 10° C. for 12 h. Water (20 mL) was added and the resulting mixture was washed with EtOAc (3×20 mL). The aqueous layer was acidified to pH 3-4 by the addition of aq. 1 N HCl. The resulting mixture was extracted with EtOAc (3×20 mL). The combined organic extracts were washed with water (20 mL), aq saturated NaCl (20 mL), dried over anhyd Na₂SO₄ and concentrated under reduced pressure to give Compound 48 as a colorless oil (290 mg, 64.4% yield) which was used in subsequent reactions without further purification.

### 1-N-[4-(6,7-Dimethoxyquinolin-4-yl)oxyphenyl]-1-N′-(4-fluorophenyl)-1-N′-(methoxymethyl)cyclopropane-1,1-dicarboxamide (49)

To a solution of Compound 48 (260 mg, 972.86 μmol, 1 eq) and Compound 3 (230.62 mg, 778.29 μmol, 0.8 eq) in pyridine (5 mL) was added EDCI (373.00 mg, 1.95 mmol, 2.0 eq). The mixture was stirred at 10° C. for 12 h. The mixture was concentrated under reduced pressure. Water (50 mL) was added and the resulting mixture was extracted with EtOAc (3×30 mL). The combined organic extracts were washed with water (20 mL), aq saturated NaCl (20 mL), dried over anhyd Na₂SO₄ and concentrated under reduced pressure. The resulting residue was purified by silica gel column chromatography (DCM/MeOH=1/0 to 5/1) followed by further purification by prep-HPLC (column: Venusil ASB Phenyl 150*30 mm*5 um; mobile phase: [water (0.05% HCl)-ACN]; B %: 30%-60%, 10 min) to give a solution. Aq saturated NaHCO₃ (2 mL) was added to the solution which was then extracted with DCM (3×20 mL). The combined organic extracts were washed with water (10 mL), aq saturated NaCl (10 mL), dried over anhyd Na₂SO₄ and concentrated under reduced pressure. The resulting residue was dissolved in a mixture of water (20 mL) and acetonitrile (MeCN) (5 mL) and the resulting solution was lyophilized to give Compound 49 as a white solid (41 mg, 72.9% yield). ¹H NMR (400 MHz, DMSO-d₆) δ 9.58 (br s, 1H), 8.48 (d, 1H), 7.51 (s, 1H), 7.46 (br d, 2H), 7.40 (s, 1H), 7.29 (br dd, 2H), 7.19-7.08 (m, 4H), 6.43 (d, 1H), 5.03 (s, 2H), 3.95 (d, 6H), 3.30 (s, 3H), 1.47-1.41 (m, 2H), 1.27 (br s, 2H); MS for C₃₀H₂₈FN₃O₆, found 546.1 (MH+).

### Example 13: 1-N′-[4-(6,7-Dimethoxyquinolin-4-yl)oxy-3-fluorophenyl]-1-N-(4-fluorophenyl)-1-N′-methylcyclopropane-1,1-dicarboxamide (51)

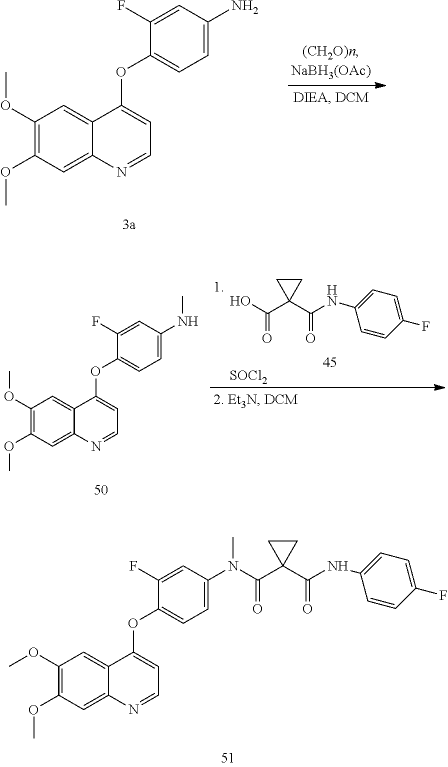

4-((6,7-Dimethoxyquinolin-4-yl)oxy)-3-fluoro-N-methylaniline (50): To a mixture of Compound 3a (200 mg, 636.31 μmol, 1 eq) and (HCHO)ₙ (3.82 mg, 1.27 mmol, 2 eq) in DCM (5 mL) was added NaBH(OAc)₃ (269.72 mg, 1.27 mmol, 2 eq) and DIEA (164.48 mg, 1.27 mmol, 221.67 μL, 2 eq). The mixture was stirred at 60° C. for 12 h. The reaction was diluted with water (10 mL) and extracted with DCM (20 mL). The organic phase was washed with aq saturated NaCl (5 mL) and concentrated to give the crude product which was then purified by silica gel column chromatography (100% Ethyl acetate in Petroleum ether) to give Compound 50 as a colorless solid (200 mg, 95.73% yield). MS for C₁₈H₁₇FN₂O₃, found 328.9 (MH+).

### 1-N′-[4-(6,7-Dimethoxyquinolin-4-yl)oxy-3-fluorophenyl]-1-N-(4-fluorophenyl)-1-N′-methylcyclopropane-1,1-dicarboxamide (51)

A solution of Compound 45 (150 mg, 672.04 μmol, 1 eq) in SOCl₂ (5 mL) was stirred at 60° C. for 1 hr. The reaction was concentrated to give the crude acid chloride of Compound 45 as a yellow solid (150 mg, 92.4% yield) which was used for the next step without purification. To a solution of Compound 50 (200 mg, 609.13 μmol, 1 eq) in DCM (10 mL) was added the above described acid chloride of Compound 45 and Et₃N (67.80 mg, 670.04 μmol, 93.26 μL, 1.1 eq). The resulting mixture was stirred at 60° C. for 12 h. After concentrating the reaction mixture, the crude product was triturated with EtOAc at 20° C., followed by trituration with MeOH at 20° C. The resulting crude product was purified by prep-HPLC (column: Agela ASB 150*25 mm*5 um; mobile phase: [water (0.05% HCl)-ACN]; B %: 32%-62%, 9 min) to give Compound 51 as a white solid (99.9 mg, 30.1% yield). ¹H NMR (400 MHz, DMSO-d₆) δ 9.92 (s, 1H), 8.89 (d, 1H), 7.82 (s, 1H), 7.69 (s, 1H), 7.50 (d, 4H), 7.32 (d, 1H), 7.08 (t, 2H), 6.74 (d, 1H), 4.03 (s, 6H), 3.32 (s, 3H), 1.48 (s, 2H), 1.30 (s, 2H); MS for C₂₉H₂₅F₂N₃O₅, found 534.1 (MH+).

### Biological Examples

### Example A: AXL Autophosphorylation ELISA in A-172 Cells

A-172 glioblastoma cells (ATCC #CRL-1620) were seeded at 2.5×10⁵ cells/well onto 24-well plates (Greiner #662165), in DMEM (Thermo Fisher #11995-040) containing 10% FBS (Thermo Fisher #26140-079), 1% MEM NEAA (Thermo Fisher #11140-050), 1% GlutaMax (Thermo Fisher #35050-061), and 1% Penicillin Streptomycin (Thermo Fisher #15140-122). A-172 cells were incubated at 37° C., 5% CO₂ for 24 h and then starved for 24 h in serum-free medium. Test compounds were serially diluted to produce an 8-point dose curve in fresh serum-free medium to a final concentration of 0.3% DMSO (vehicle) and added to the cells and incubated for 1 h. Cells were then stimulated with 1 μg/mL recombinant human Gas6 (R&D Systems #885-GSB-500) for 15 min, washed with cold PBS, and immediately lysed with 150 μL of cold IX lysis buffer [20 mM Tris, 137 mM sodium chloride, 2 mM EDTA, 10% glycerol, 1% NP-40 alternative, 1 mM activated sodium orthovanadate, 1 mM PefaBloc SC (Sigma-Aldrich #11429868001), protease/phosphatase inhibitor tablet (Thermo Fisher #A32959)]. Lysates were collected and 100 μL/well added into the human phospho-AXL DuoSet IC ELISA (R&D Systems #DYC2228-2). Assay was performed according to manufacturer's instructions and sample phospho-AXL concentrations were extrapolated using human phospho-AXL control (R&D Systems #841645) as a standard. Positive control wells (100% activity) contained Gas6-stimulated, DMSO-treated cell lysates. Negative control wells (0% activity) contained Gas6-stimulated, reference inhibitor-treated cell lysates. IC₅₀ values were calculated by nonlinear regression analysis using a 4-parameter logistic curve fit in ActivityBase XE (IDBS).

### Example B: Met Autophosphorylation ELISA in PC-3 Cells

PC-3 prostate cancer cells (ATCC #CRL-1435) were seeded at 4×10⁴ cells/well onto 24-well plates (Greiner #662165), in DMEM (Thermo Fisher #11995-040) containing 10% FBS (Thermo Fisher #26140-079), 1% MEM NEAA (Thermo Fisher #11140-050), 1% GlutaMax (Thermo Fisher #35050-061), and 1% Penicillin Streptomycin (Thermo Fisher #15140-122). PC-3 cells were incubated at 37° C., 5% CO₂ for 24 h and then starved for 3 h in serum-free medium. Test compounds were serially diluted to produce an 8-point dose curve in fresh serum-free medium to a final concentration of 0.3% DMSO (vehicle) and added to the cells and incubated for 1 h. Cells were then stimulated with 100 ng/mL recombinant human HGF (R&D Systems #294-HG-250) for 10 min, washed with cold PBS, and immediately lysed with 130 μL of cold IX lysis buffer [20 mM Tris, 137 mM sodium chloride, 2 mM EDTA, 10% glycerol, 1% NP-40 alternative, 1 mM activated sodium orthovanadate, 1 mM PefaBloc SC (Sigma-Aldrich #11429868001), protease/phosphatase inhibitor tablet (Thermo Fisher #A32959)]. Lysates were clarified by centrifugation and 100 μL/well added into the PathScan phospho-Met (panTyr) Sandwich ELISA (Cell Signaling Technology #7333). Assay was performed according to manufacturer's instructions. Positive control wells (100% activity) contained HGF-stimulated, DMSO-treated cell lysates. Negative control wells (0% activity) contained HGF-stimulated, reference inhibitor-treated cell lysates. IC50 values were calculated by nonlinear regression analysis using a 4-parameter logistic curve fit in ActivityBase XE (IDBS).

### Example C: KDR Autophosphorylation ELISA in HUVEC Cells

Human

umbilical vein endothelial cells or HUVEC (Lonza #C2519A) were seeded at 2×10⁴ cells/well onto 96-well plates (Corning #3904), in EGM-2 growth medium (Lonza #CC-3162) containing 1% Penicillin Streptomycin (Thermo Fisher #15140-122). HUVEC cells were incubated at 37° C., 5% CO₂ for 24 h and then starved for 24 h in serum-free EBM-2 basal medium (Lonza #CC-3156) containing 1% Penicillin Streptomycin. Test compounds were serially diluted to produce an 8-point dose curve in fresh serum-free medium to a final concentration of 0.3% DMSO (vehicle) and added to the cells and incubated for 1 h. Cells were then stimulated with 100 ng/mL recombinant human VEGF165 (R&D Systems #293-VE-500) for 5 min, washed with cold PBS, and immediately lysed with 130 μL of cold IX lysis buffer [20 mM Tris, 137 mM sodium chloride, 2 mM EDTA, 10% glycerol, 1% NP-40 alternative, 1 mM activated sodium orthovanadate, 1 mM PefaBloc SC (Sigma-Aldrich #11429868001), protease/phosphatase inhibitor tablet (Thermo Fisher #A32959)]. Lysates were collected and 100 μL/well added into the human phospho-KDR DuoSet IC ELISA (R&D Systems #DYC 1766-2). Assay was performed according to manufacturer's instructions and sample phospho-KDR concentrations were extrapolated using human phospho-KDR control (R&D Systems #841421) as a standard. Positive control wells (100% activity) contained VEGF165-stimulated, DMSO-treated cell lysates. Negative control wells (0% activity) contained non-stimulated cell lysates. IC₅₀ values were calculated by nonlinear regression analysis using a 4-parameter logistic curve fit in ActivityBase XE (IDBS).

### Example D: Mer Autophosphorylation ELISA in Transient Transfected 293A Cells

293A cells (Thermo Fisher #R70507) were seeded at 1.5×10⁶ cells/well onto 100 mm dish (Greiner #664169), in DMEM (Thermo Fisher #11995-040) containing 10% FBS (Thermo Fisher #26140-079), 1% MEM NEAA (Thermo Fisher #11140-050), 1% GlutaMax (Thermo Fisher #35050-061), and 1% Penicillin Streptomycin (Thermo Fisher #15140-122). 293A cells were incubated at 37° C., 5% CO₂ for 24 h and then transfected with 6 μg MERTK DNA (Genecopoeia #EX-Z8208-M02) using TransIT LT1 transfection reagent (Mirus-Bio #MIR2305). After 24 h incubation, the transfected 293A cells were seeded at 1×10⁵ cells/well onto 96-well plates (Corning #3904) in DMEM growth medium overnight. Test compounds were serially diluted to produce an 8-point dose curve in fresh serum-free medium to a final concentration of 0.3% DMSO (vehicle) and added to the cells and incubated for 1 h. Cells were then immediately lysed with 150 μL of cold IX lysis buffer [20 mM Tris, 137 mM sodium chloride, 2 mM EDTA, 10% glycerol, l % NP-40 alternative, 1 mM activated sodium orthovanadate, 1 mM PefaBloc SC (Sigma-Aldrich #11429868001), protease/phosphatase inhibitor tablet (Thermo Fisher #A32959)]. Lysates were clarified by centrifugation and 50 μL/well added into the human phospho-Mer DuoSet IC ELISA (R&D Systems #DYC2579-2). Assay was performed according to manufacturer's instructions and sample phospho-Mer concentrations were extrapolated using human phospho-Mer control (R&D Systems #841793) as a standard. Positive control wells (100% activity) contained DMSO-treated cell lysates. Negative control wells (0% activity) contained reference inhibitor-treated cell lysates. IC₅₀ values were calculated by nonlinear regression analysis using a 4-parameter logistic curve fit in ActivityBase XE (IDBS).

### Example E

Compounds of the present disclosure, as exemplified herein, were tested in the assays of Examples A, B, C, and D and showed IC₅₀ values in the following ranges: A: IC₅₀≤10 nM; B: 10 nM<IC₅₀≤100 nM; C: 100 nM<IC₅₀≤300 nM; D: IC₅₀>300 nM. “NT” means not tested. Results are provided in Table 2.

TABLE 2
Biological Activities of Selected Compounds

| | | Axl | Mer | c-Met | KDR |
|---|---|---|---|---|---|
| | | IC₅₀ | IC₅₀ | IC₅₀ | IC₅₀ |
| # | Structure | (nM) | (nM) | (nM) | (nM) |
| 9 | 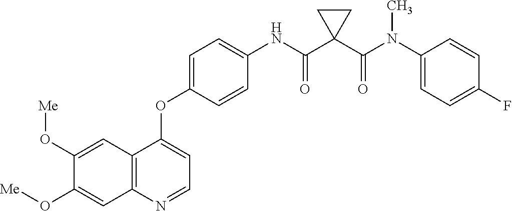 [SMILES: COc1cc2nccc(Oc3ccc(NC(=O)C4(C(=O)N(C)c5ccc(F)cc5)CC4)cc3)c2cc1OC] | D | D | D | D |
| 10 | 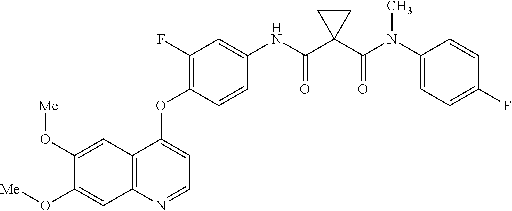 [SMILES: COc1cc2nccc(Oc3ccc(NC(=O)C4(C(=O)N(C)c5ccc(F)cc5)CC4)cc3F)c2cc1OC] | D | D | D | D |
| 11 | 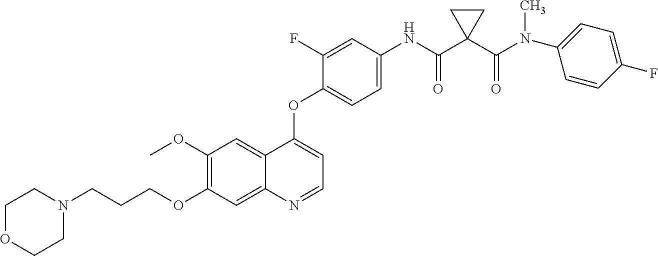 [SMILES: COc1cc2c(Oc3ccc(NC(=O)C4(C(=O)N(C)c5ccc(F)cc5)CC4)cc3F)ccnc2cc1OCCCN1CCOCC1] | NT | NT | D | D |
| 34 | 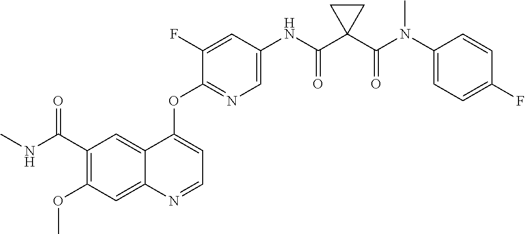 [SMILES: CNC(=O)c1cc2c(Oc3ncc(NC(=O)C4(C(=O)N(C)c5ccc(F)cc5)CC4)cc3F)ccnc2cc1OC] | D | D | D | D |
| 35 | 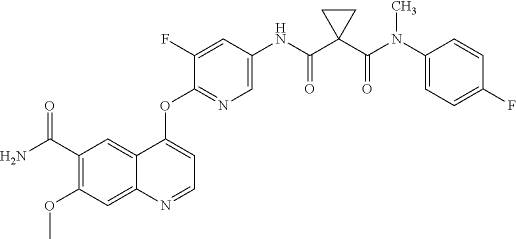 [SMILES: COc1cc2nccc(Oc3ncc(NC(=O)C4(C(=O)N(C)c5ccc(F)cc5)CC4)cc3F)c2cc1C(N)=O] | D | D | D | D |
| 44 | 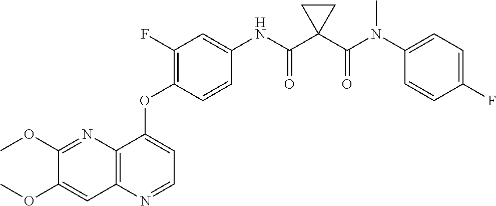 [SMILES: COc1cc2nccc(Oc3ccc(NC(=O)C4(C(=O)N(C)c5ccc(F)cc5)CC4)cc3F)c2nc1OC] | D | NT | D | D |
| 49 | 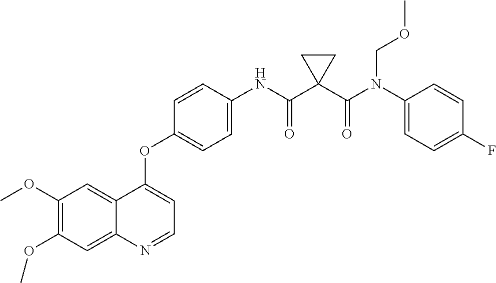 [SMILES: COCN(C(=O)C1(C(=O)Nc2ccc(Oc3ccnc4cc(OC)c(OC)cc34)cc2)CC1)c1ccc(F)cc1] | NT | NT | NT | NT |
| 51 | 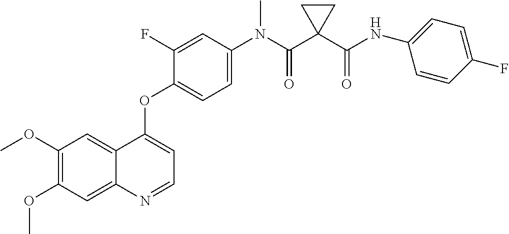 [SMILES: COc1cc2nccc(Oc3ccc(N(C)C(=O)C4(C(=O)Nc5ccc(F)cc5)CC4)cc3F)c2cc1OC] | D | D | D | NT |

### Example F: Microsomal Assay

Liver microsomes tissue fractions were used for in vitro assessment of metabolic stability of compounds by cytochrome P450 (CYP450) (for example, CYP3A4, CYP2C9) mediated phase I oxidation, and metabolism through other pathways. Human, mouse, rat, and dog liver microsomes tissue fractions were obtained from Corning Gentest and BioreclamationIVT.

The assay was carried out in 96-well microtiter plates. Compounds were incubated (N=l) at 37° C. in the presence of liver microsomes. Reaction mixtures (25 μL) contained a final concentration of 1 μM test compound, 0.5 mg/mL liver microsomes (LM) protein, and 1 mM NADPH in 100 mM potassium phosphate, pH 7.4 buffer with 3.3 mM MgCl₂. The extent of metabolism was calculated as the disappearance of the test compound, compared to the 0-min control reaction incubations. Verapamil was included as a positive control to verily assay performance.

At each of the four time points, 150 μL of quench solution (100% acetonitrile with 0.1% formic acid) with internal standard (bucetin for positive ESI mode) was transferred to each well. Plates were sealed and centrifuged at 10° C. for 15 minutes at 4000 rpm. The supernatant was transferred to fresh plates for LC/MS/MS analysis.

All samples were analyzed on LC/MS/MS using an AB Sciex API 4000 instrument, coupled to a Shimadzu LC-20AD LC Pump system. Analytical samples were separated using a Waters Atlantis T3 dC18 reverse phase HPLC column (20 mm×2.1 mm) at a flow rate of 0.5 mL/min. The mobile phase consisted of 0.1% formic acid in water (solvent A) and 0.1% formic acid in 100% acetonitrile (solvent B). Assay conditions are summarized in Table 3. Elution conditions are detailed in Table 4.

TABLE 3
Assay conditions

| | |
|---|---|
| [Compound] | 1 μM |
| [LM] | 0.5 mg/mL |
| [NADPH] | 1 mM |
| Buffer | 100 mM Potassium Phosphate, pH 7.4, with 3.3 mM MgCl₂ |
| Time | 0, 15, 30, and 60 min |
| Temperature | 37° C. |

TABLE 4
Elution conditions

| Time (min) | Flow (μL/min) | % A | % B |
|---|---|---|---|
| 0 | 500 | 98 | 2 |
| 0.3 | 500 | 98 | 2 |
| 1.3 | 500 | 2 | 98 |
| 1.7 | 500 | 2 | 98 |
| 1.71 | 500 | 98 | 2 |
| 2.5 | 500 | 98 | 2 |

Results:

Compounds of the present disclosure, as exemplified herein, were tested in the assay of this Example F. Metabolic stability results as calculated intrinsic clearance and t1/2 values of test compounds in liver microsomes are listed in Table 5. Reference compound verapamil behaved as expected.

TABLE 5
Microsomal Stability Data

| | Human Liver | | Mouse Liver | | Rat Liver | |
|---|---|---|---|---|---|---|
| | Microsome | | Microsome | | Microsome | |
| | Stability | | Stability | | Stability | |
| | | CLint | | CLint | | CLint |
| | | (μL/min/ | | (μL/min/ | | (μL/min/ |
| Cpd. | t½ | million | t½ | million | t½ | million |
| # | (min) | cells) | (min) | cells) | (min) | cells) |
| Verapamil | 15.2 | 91 | 7.4 | 188 | 4.2 | 332 |
| 9 | 1.4 | 1013 | 3.1 | 441 | 1.4 | 999 |
| 10 | 1.3 | 1065 | 4.1 | 339 | 1.3 | 1039 |
| 11 | 1.6 | 893 | 7.3 | 189 | 1.6 | 882 |
| 34 | 1.3 | 1049 | 2.6 | 538 | 1.3 | 1041 |
| 35 | 1.3 | 1077 | 1.9 | 744 | 1.2 | 1129 |
| 49 | 1.3 | 1052 | 1.49 | 928 | 1.35 | 1028 |
| 51 | 29.7 | 47.0 | 9.6 | 145 | 14.3 | 97.0 |

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A compound according to formula I′: 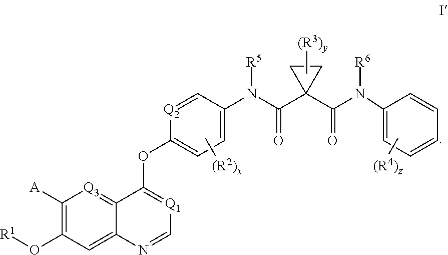
or a pharmaceutically acceptable salt thereof, wherein
A is a C₁₋₆ alkoxy, or C(O)NR⁷R⁸;
R¹ is C₁₋₆ alkyl or heterocycloalkyl-C₁₋₆ alkylene-;
R² is halo;
R³ is halo, OH, C₁₋₄ alkoxy, or CF₃;
R⁴ is halo;
one of R⁵ and R⁶ is —CHR′R″ and the other of R⁵ and R⁶ is H or —CHR′R″;
R⁷ and R⁸ are each independently H or a C₁₋₆ alkyl;
each of R′ and R″ is independently selected from the group consisting of H, OH and C₁₋₆ alkoxy;
Q₁, Q₂, and Q₃ are each independently CH or N;
x is 0, 1, 2, 3, or 4;
y is 0, 1, 2, 3, or 4; and
z is 0, 1, 2, 3, 4, or 5.

2. The compound of claim 1 or a pharmaceutically acceptable salt thereof, wherein R¹ is C₁₋₆ alkyl or

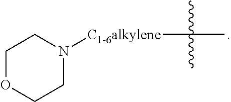

3. The compound of claim 1 or a pharmaceutically acceptable salt thereof, wherein R¹ is C₁₋₆ alkyl.

4. The compound of claim 3 or a pharmaceutically acceptable salt thereof, wherein R¹ is methyl.

5. The compound of claim 1 or a pharmaceutically acceptable salt thereof, wherein R¹ is

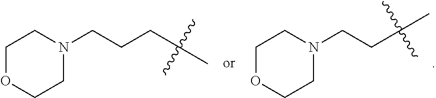 [SMILES: *CCCN1CCOCC1 ; *CCN1CCOCC1]

6. The compound of any one of claims 1-5 or a pharmaceutically acceptable salt thereof, wherein R², R³, and R⁴, are each independently F.

7. The compound of any one of claims 1-6 or a pharmaceutically acceptable salt thereof, wherein x, y, and z are each independently 0 or 1.

8. The compound of any one of claims 1-7 or a pharmaceutically acceptable salt thereof, wherein Q₁ and Q₂ are each CH.

9. The compound of any one of claims 1-8 or a pharmaceutically acceptable salt thereof, wherein one of R⁵ and R⁶ is —CHR′R″ and the other is H.

10. The compound of claim 9 or a pharmaceutically acceptable salt thereof, wherein R⁵ is —CHR′R″.

11. The compound of claim 10 or a pharmaceutically acceptable salt thereof, wherein R⁵ is methyl.

12. The compound of claim 10 or a pharmaceutically acceptable salt thereof, wherein R⁵ is —CH₂OH or —CH₂OCH₃.

13. The compound of claim 9 or a pharmaceutically acceptable salt thereof, wherein R⁶ is —CHR′R″.

14. The compound of claim 13 or a pharmaceutically acceptable salt thereof, wherein R⁶ is methyl.

15. The compound of claim 13 or a pharmaceutically acceptable salt thereof, wherein R⁶ is —CH₂OH or —CH₂OCH₃.

16. The compound according to any one of claims 1-15, wherein A is C₁₋₆ alkoxy.

17. The compound according to claim 16, wherein A is methoxy, ethoxy, n-propoxy, isopropoxy, butoxy, or t-butoxy.

18. The compound according to claim 17, wherein A is methoxy.

19. The compound according to claim 1, having formula Ilia:

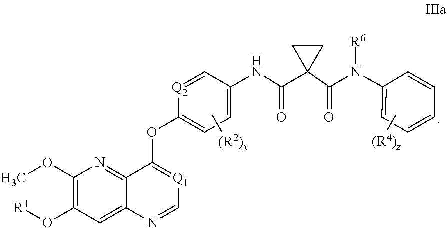

20. The compound according to any one of claims 1-15, wherein A is C(O)NR⁷R⁸.

21. The compound according to claim 20, wherein one of R⁷ and R⁸ is H, and the other is a C₁₋₆ alkyl.

22. The compound according to claim 21, wherein one of R⁷ and R⁸ is H, and the other is methyl.

23. The compound according to claim 20, wherein both R⁷ and R⁸ are H.

24. The compound according to claim 1, having formula IIIb:

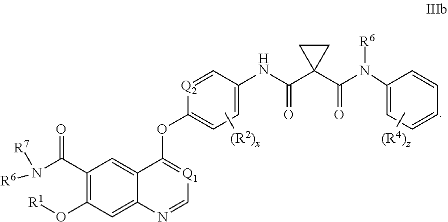

25. The compound according to any one of claims 1-24, wherein x is 1, 2, 3, or 4.

26. The compound according to claim 25, wherein R² is F.

27. The compound according to any one of claims 1-26, wherein R⁴ is F, and z is 1, 2, 3, or 4.

28. The compound according to claim 27, wherein the moiety

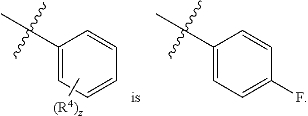

29. The compound according to any one of claims 1-18 and 20-28, wherein Q₁; Q₂, and Q₃ are each CH.

30. The compound according to any one of claims 1-18 and 20-28, wherein Q₁; and Q₃ are each CH, and Q₂ is N.

31. The compound according to any one of claims 1-23 and 25-28, wherein Q₁; and Q₂ are each CH, and Q₃ is N.

32. A compound of Formula I 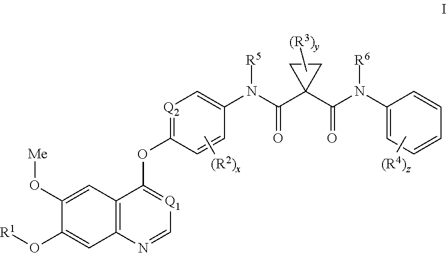
or a pharmaceutically acceptable salt thereof, wherein:
R¹ is C₁₋₆ alkyl or heterocycloalkyl-C₁₋₆ alkylene-;
R² is halo;
R³ is halo, OH, C₁₋₄ alkoxy, or CF₃;
R⁴ is halo;
one of R⁵ and R⁶ is —CHR′R″ and the other of R⁵ and R⁶ is H or —CHR′R″;
each of R′ and R″ is independently selected from the group consisting of H, OH and C₁₋₆ alkoxy;
Q₁ and Q₂ are each independently CH or N;
x is 0, 1, 2, 3, or 4;
y is 0, 1, 2, 3, or 4; and
z is 0, 1, 2, 3, 4, or 5.

33. The compound of claim 32 or a pharmaceutically acceptable salt thereof, having formula IA:

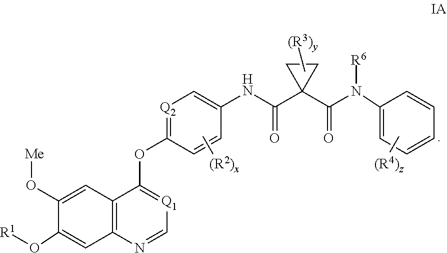

34. The compound of claim 32 or a pharmaceutically acceptable salt thereof, having formula IB:

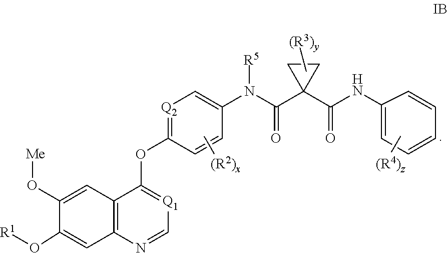

35. The compound of claim 32 or a pharmaceutically acceptable salt thereof, having formula II 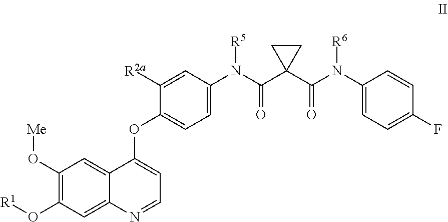
wherein R^{2a} is H or halo.

36. The compound of claim 35 or a pharmaceutically acceptable salt thereof, wherein R¹ is C₁₋₆ alkyl or

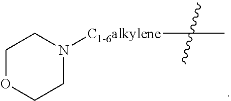

37. The compound of claim 35 or a pharmaceutically acceptable salt thereof, wherein R¹ is C₁₋₆ alkyl.

38. The compound of claim 37 or a pharmaceutically acceptable salt thereof, wherein R¹ is methyl.

39. The compound of claim 35 or a pharmaceutically acceptable salt thereof, wherein R¹ is

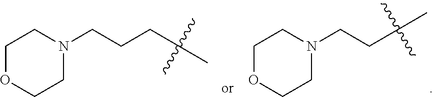 [SMILES: *CCCN1CCOCC1 ; *CCN1CCOCC1]

40. The compound of any one of claims 35-39 or a pharmaceutically acceptable salt thereof, wherein R^{2a} is H or F.

41. The compound of any one of claims 35-40 or a pharmaceutically acceptable salt thereof, wherein one of R⁵ and R⁶ is —CHR′R″ and the other is H.

42. The compound of claim 41 or a pharmaceutically acceptable salt thereof, wherein R⁵ is —CHR′R″.

43. The compound of claim 42 or a pharmaceutically acceptable salt thereof, wherein R⁵ is methyl.

44. The compound of claim 42 or a pharmaceutically acceptable salt thereof, wherein R⁵ is —CH₂OH or —CH₂OCH₃.

45. The compound of claim 41 or a pharmaceutically acceptable salt thereof, wherein R⁶ is —CHR′R″.

46. The compound of claim 45 or a pharmaceutically acceptable salt thereof, wherein R⁶ is methyl.

47. The compound of claim 45 or a pharmaceutically acceptable salt thereof, wherein R⁶ is —CH₂OH or —CH₂OCH₃.

48. The compound of any one of claims 35-40 or a pharmaceutically acceptable salt thereof, having formula IIA:

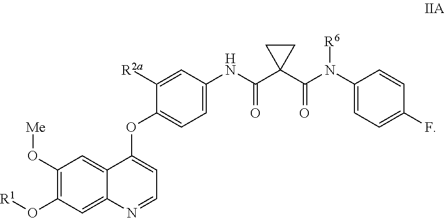

49. The compound of any one of claims 35-40 or a pharmaceutically acceptable salt thereof, having formula IIB:

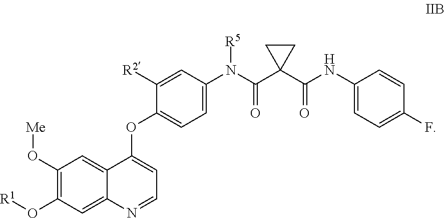

50. A compound of claim 1, selected from:
N-(4-((6,7-dimethoxyquinolin-4-yl)oxy)phenyl)-N-(4-fluorophenyl)-N-methylcyclopropane-1,1-dicarboxamide;
N-(4-((6,7-dimethoxyquinolin-4-yl)oxy)-3-fluorophenyl)-N-(4-fluorophenyl)-N-methylcyclopropane-1,1-dicarboxamide;
N-(3-fluoro-4-((6-methoxy-7-(3-morpholinopropoxy)quinolin-4-yl)oxy)phenyl)-N-(4-fluorophenyl)-N-methylcyclopropane-1,1-dicarboxamide;
N-(4-((6,7-dimethoxyquinolin-4-yl)oxy)phenyl)-N-(4-fluorophenyl)-N-(hydroxymethyl)cyclopropane-1,1-di carboxamide;
N-(4-((6,7-dimethoxyquinolin-4-yl)oxy)-3-fluorophenyl)-N-(4-fluorophenyl)-N-(hydroxymethyl)cyclopropane-1,1-di carboxamide;
N-(3-fluoro-4-((6-methoxy-7-(3-morpholinopropoxy)quinolin-4-yl)oxy)phenyl)-N-(4-fluorophenyl)-N-(hydroxymethyl)cyclopropane-1,1-dicarboxamide;
1-N-[5-fluoro-6-[7-methoxy-6-(methylcarbamoyl)quinolin-4-yl]oxypyridin-3-yl]-1-N′-(4-fluorophenyl)-1-N′-methylcyclopropane-1,1-dicarboxamide;
N-(5-fluoro-6-((7-methoxy-6-(methylcarbamoyl)quinolin-4-yl)oxy)pyridin-3-yl)-N-(4-fluorophenyl)-N-(hydroxymethyl)cyclopropane-1,1-dicarboxamide;
1-N-[6-(6-carbamoyl-7-methoxyquinolin-4-yl)oxy-5-fluoropyridin-3-yl]-1-N′-(4-fluorophenyl)-1-N′-methylcyclopropane-1,1-dicarboxamide;
N-(6-((6-carbamoyl-7-methoxyquinolin-4-yl)oxy)-5-fluoropyridin-3-yl)-N-(4-fluorophenyl)-N-(hydroxymethyl)cyclopropane-1,1-dicarboxamide;
1-N-[4-[(6,7-dimethoxy-1,5-naphthyridin-4-yl)oxy]-3-fluorophenyl]-1-N′-(4-fluorophenyl)-1-N′-methylcyclopropane-1,1-dicarboxamide;
N-(4-((6,7-dimethoxy-1,5-naphthyridin-4-yl)oxy)-3-fluorophenyl)-N-(4-fluorophenyl)-N-(hydroxymethyl)cyclopropane-1,1-dicarboxamide;
1-N-[4-(6,7-dimethoxy quinolin-4-yl)oxyphenyl]-1-N′-(4-fluorophenyl)-1-N′-(methoxymethyl)cyclopropane-1,1-dicarboxamide; or
1-N′-[4-(6,7-dimethoxyquinolin-4-yl)oxy-3-fluorophenyl]-1-N-(4-fluorophenyl)-1-N′-methylcyclopropane-1,1-dicarboxamide;
or a pharmaceutically acceptable salt thereof.

51. A pharmaceutical composition comprising a compound according to any one of claims 1-50, or a pharmaceutically acceptable salt thereof, and a pharmaceutically acceptable carrier or excipient.

52. A method of treating a disease, disorder, or syndrome mediated at least in part by modulating in vivo activity of a protein kinase in a patient, comprising administering to the patient in need thereof a compound of any of claims 1-50 or a pharmaceutical composition of claim 51.

53. The method of claim 52, wherein the disease, disorder, or syndrome mediated at least in part by modulating in vivo activity of a protein kinase is cancer.

54. A method for inhibiting a protein kinase, the method comprising contacting the protein kinase with a compound of any one of claims 1-50.

55. The method of any one of claims 52-54, wherein the protein kinase is Axl, Mer, c-Met, KDR, or a combination thereof.
